(19) United States
(12) Patent Application Publication
(10) Pub. No.: US 2026/0240480 A1
(43) Pub. Date: Aug. 20, 2026

(54) ELECTROCARDIOGRAM MEASUREMENT APPARATUS

(71) Applicant: HEXACHECK INC., Daejeon (KR)

(72) Inventor: In Duk HWANG, Sejong (KR)

(73) Assignee: HEXACHECK INC., Daejeon (KR)

(21) Appl. No.: 19/160,797

(22) PCT Filed: Feb. 28, 2024
(86) PCT No.: PCT/KR2024/002615

§ 371 Date: Nov. 3, 2025

(30) Foreign Application Priority Data
Feb. 28, 2023 (KR) 10-2023-0027393

(51) Int. Cl. A61B 5/339; A61B 5/00; A61B 5/024; A61B 5/308

(52) U.S. Cl. CPC A61B 5/339; A61B 5/0006; A61B 5/02416; A61B 5/308; A61B 5/681; A61B 5/746; A61B 5/6828

(57) ABSTRACT

An electrocardiogram measurement apparatus according to the present invention comprises: first and second electrodes receiving two electrocardiogram voltages of contact body parts, respectively; two amplifiers receiving the two electrocardiogram voltages from the first electrode and the second electrode, respectively; one electrode driver that outputs a driving voltage; and a third electrode that transmits an electrocardiogram voltage of a human body part in contact with an input terminal of the electrode driver, wherein each of the two amplifiers simultaneously receives and amplifies one electrocardiogram voltage, and the three electrodes are in contact with the right hand, the left hand, and the left leg, thereby enabling the apparatus according to the present invention to obtain six electrocardiogram lead signals.

### TECHNICAL FIELD

An electrocardiogram system provides a waveform of an electrical signal, namely, an electrocardiogram, which contains very useful but easily obtainable information to analyze the condition of a patient's heart. It can be said that the electrocardiogram system includes an electrocardiogram measurement apparatus (a measurement sensor) and a computer. In these days, almost all individuals use a smartphone. A smartphone can be considered a computer capable of wireless communication and providing a good display. Therefore, the combination of the electrocardiogram measurement apparatus (measurement sensor) and the smartphone can be a good electrocardiogram system. The present invention relates to an electrocardiogram measurement apparatus (measurement sensor) that an individual can use in association with a smartphone. According to International Patent Classification (IPC), the apparatus for measuring an electrocardiogram according to the present invention is classified into class A61B 5/04 to which detecting, measuring or recording bioelectric signals of the body or parts thereof belongs.

### BACKGROUND ART

An electrocardiogram system is a useful apparatus capable of conveniently diagnosing a patient's heart condition. Electrocardiograms can be classified into several types depending on the purpose thereof. The standard of hospital electrocardiography systems which are used to obtain as much information as possible is a 12-channel electrocardiogram system employing 10 wet electrodes. A patient monitoring system is used to continuously measure a patient's heart condition with a small number of wet electrodes attached to the patient's body. A Holter recorder and an event recorder that a user can use by themselves while moving around have the following essential features. These features include a compact size, battery-powered operation, a storage device provided to store measured data, and a communication device capable of transmitting the data. The Holter recorder usually uses 4 to 6 wet electrodes and cables connected to the electrodes, and provides a multi-channel ECG. However, the user feels uncomfortable about the Holter recorder because the wet electrodes connected to the cables are attached to the body. Recently released electrocardiogram systems such as a patch type system also require electrodes to be kept attached to the body.

The event recorder allows users to carry the recorder and measure ECG on their own when they feel an abnormality in their heart. Therefore, the event recorder is compact and does not have a cable for connecting electrodes, and dry electrodes are provided on the surface of the event recorder. The conventional event recorder is a 1-channel electrocardiography system, i.e., a 1-lead electrocardiogram system that measures one ECG signal while both hands of a user are in contact with two electrodes.

An electrocardiogram measurement apparatus which is sought or required by the present invention is required to be convenient for personal use, to provide accurate and abundant electrocardiogram measurements, and to be compact so as to be easily carried. The required apparatus for convenient personal use should be able to transmit data via wireless communication to a smartphone. To this end, the required apparatus should be battery-powered. To increase battery life while obtaining a compact size of the apparatus, the required apparatus should not include a display, and the electrocardiogram should be displayed on a smartphone.

In the present invention, in order to provide accurate and abundant ECG measurements, two limb leads are directly measured at the same time. As described later, in the present invention, four leads can be calculated and provided based on the two limb lead measurements performed simultaneously. Conventionally, regarding an electrocardiogram, “channel” and “lead” are used interchangeably to mean one ECG signal or ECG voltage. Regarding an electrocardiogram, the word “simultaneously” should be used very carefully. The phrase “simultaneously” means that operations are not “sequential”. In other words, measuring two leads simultaneously should literally mean measuring two ECG voltages substantially at one moment. Specifically, when lead II is sampled while the voltage of lead I is sampled with a constant sampling period, measurements can be said to be performed simultaneously only if sampling lead II is performed within a shorter time than the sampling period from each time of sampling Lead I. The word “measurement” should also be used carefully. The word “measurement” should be mentioned only when a physical quantity is actually measured. In digital measurement, one measurement should mean one AD conversion. As will be described later, for example, by measuring lead I and lead III in electrocardiogram measurement, lead II can be calculated according to Kirchhoff's voltage law. In this case, lead II must be expressed as “calculated,” not “measured,” which can cause confusion.

One of the most difficult challenges in electrocardiogram measurement is to remove power line interference included in the electrocardiogram signal. A well-known method for removing power line interference is Driven Right Leg (DRL). Substantially, almost all electrocardiograms remove power line interference by the DRL. A drawback of the DRL is that one DRL electrode should be attached to the right leg or a lower right part of a torso. Therefore, in order to measure two limb leads using the DRL technique, conventional technology requires four electrodes including the DRL electrode to be brought into contact with the body. However, an important issue raised at this time is that a cable must be used or the size of the apparatus is increased because the DRL electrode should be brought into contact with the lower right abdomen. In other words, it is difficult to scale down an electrocardiogram measurement apparatus configured to measure two leads using a DRL electrode to the size of a credit card. Another important issue is that if the DRL electrode is arranged adjacent to another electrode and brought into contact with the human body, the voltage of the adjacent electrode is distorted because the voltage of the DRL electrode includes components of an electrocardiogram signal. Removing power line interference without using the DRL electrode is very difficult and requires use of a special circuit (In-Duk Hwang and John G. Webster, Direct Interference Cancelling for Two-Electrode Biopotential Amplifier, IEEE Transaction on Biomedical Engineering, Vol. 55, No. 11, pp. 2620-2627, 2008). In order to remove the power line interference, multiple filters having a significantly high quality factor (Q) may be required, and manufacturing and calibration of the multiple filters may be difficult.

The electrode impedance of a dry electrode is large, and accordingly the dry electrode generates greater power line interference. However, in the electrocardiogram measurement, for user convenience, it is necessary to use a dry electrode attached to the case surface of an electrocardiogram measurement apparatus without using a wet electrode connected to a cable. In addition, it is necessary to reduce the number of dry electrodes for user convenience. It is also required not to bring the DRL electrode into contact with the right leg or a lower right part of a torso. However, in the conventional technology, it is difficult to provide an electrocardiogram measurement apparatus that removes power line interference with a minimum number of electrodes and does not use a cable.

In order to solve the above problems and necessities, the present invention uses dry electrodes and does not use a cable for user convenience. To measure two limb leads simultaneously, the present invention uses two amplifiers, one electrode driver, and three electrodes connected thereto. The electrocardiogram apparatus according to the present invention provides a plate-shaped electrocardiogram apparatus having two dry electrodes separated from each other on one surface and one dry electrode on the other surface for user convenience. In addition, the present invention provides a method for removing power line interference in order not to use a DRL electrode.

As will be described later, the present invention discloses an electrocardiogram measurement apparatus including three electrodes, wherein the power line interference current flows concentrated through one electrode connected to the electrode driver, and two amplifiers connected to the other two electrodes among the three electrodes each amplify one electrocardiogram signal to measure two electrocardiogram signals simultaneously. Here, one amplifier serves to amplify one signal. In an actual configuration, one amplifier may represent a set composed of multiple cascaded amplification stages or active filters.

As described below, the conventional technology has failed to provide a technical solution provided by the present invention.

Righter (U.S. Pat. No. 5,191,891, 1993) discloses a watch-type device equipped with three electrodes. This device obtains only one ECG signal.

Amluck (DE 201 19965, 2002) discloses an electrocardiogram apparatus provided with two electrodes on the top and one electrode on the bottom. This apparatus measures only one lead. In addition, unlike the present invention, Amluck has a display and input/output buttons.

Wei et al. (U.S. Pat. No. 6,721,591, 2004) discloses that six electrodes including the ground electrode and RL electrode are used. Wei et al. discloses a method of measuring 4 leads and calculating the remaining 8 leads.

Kazuhiro (JP2007195690, 2007) discloses an apparatus equipped with a display and four electrodes including a ground electrode.

Tso (US Pub. No. 2008/0114221, 2008) discloses a meter including three electrodes. However, according to Tso, two electrodes are touched simultaneously with one hand to measure one limb lead, for example, lead I. Since one lead is measured at a time in this way, three measurements need to be performed sequentially to obtain three limb leads. In addition, according to Tso, even an augmented limb lead, which does not need to be measured directly, is directly measured and a separate platform is used for this measurement.

Chan et al. (US Pub. No. 2010/0076331, 2010) disclose a watch including three electrodes. However, according to Cho et al., three leads are measured using three differential amplifiers. In addition, Chan et al. uses three filters connected to each of the amplifiers to reduce the noise in a signal.

Bojovic et al. (U.S. Pat. No. 7,647,093, 2010) discloses a method for calculating 12 lead signals by measuring three special (non-standard) leads. However, to measure three leads, consisting of one limb lead (lead I) and two special (non-standard) leads obtained from a chest, five electrodes including one ground electrode, on both sides of a plate-shaped apparatus, and three amplifiers are provided.

Saldivar (US Pub. No. 2011/0306859, 2011) discloses a cellular phone cradle. Saldivar discloses that three electrodes are provided on one side of the cradle. However, Saldivar uses a lead selector and one differential amplifier 68 connected to two of the three electrodes to measure one lead sequentially (see FIG. 4C and paragraph [0054]). That is, according to Saldivar, 3 leads are measured sequentially one at a time.

Berkner et al. (U.S. Pat. No. 8,903,477, 2014) relates to a method of calculating 12 lead signals through sequential measurements carried out by sequentially moving an apparatus using 3 or 4 electrodes disposed on both sides of a housing. However, Berkner et al. does not disclose the detailed structure and shape of the apparatus, including how each electrode is connected internally. Most importantly, Berkner employs one amplifier 316 and one filter module 304. When one amplifier 316 and one filter module 304 are used, for example, measuring two leads requires two measurements to be performed sequentially. Specifically, Berkner discloses “ . . . so in a system comprising only 3 electrodes, the reference electrode is different and shifts for each lead measurement. This may be done by designated software and/or hardware optionally comprising a switch.” The above technique indicates that Berkner uses one amplifier 316 and one filter 304 to measure one lead at a time and performs multiple measurements sequentially. That is, the method of Berkner et al. has many disadvantages compared to the method of measuring two leads simultaneously using three electrodes and two amplifiers as presented in the present invention.

Amital (US Pub. No. 2014/0163349, 2014) discloses that a common mode cancellation signal is generated from three electrodes in an apparatus provided with four electrodes and a common mode signal is removed by coupling the common mode cancellation signal to the other electrode (see claim 1). This technique is a traditional DRL method well known before Amital.

Thomson et al. (US Pub. No. 2015/0018660, 2015) disclose a smartphone case with three electrodes attached. The smartphone case of Thomson has a hole in the front such that the smartphone screen can be seen. However, it fails to present a method for measuring two leads simultaneously using two amplifiers. In addition, since the apparatus of Thomson uses ultrasonic communication, a communication-related issue can be raised if the smartphone and the apparatus are separated by even a slight distance (about 1 foot). Further, if the user changes one's smartphone the user may not be allowed to use the existing Thomson's smartphone case.

Drake (US Pub. No. 2016/0135701, 2016) discloses that three electrodes are provided on one side of a mobile device to provide 6 leads. However, Drake discloses “comprises one or more amplifiers configured to amplify analog signals received from the three electrodes” (see paragraph [0025] and claim 4). Therefore, Drake is not clear about a key part of the invention: how many amplifiers are used and how the amplifiers are connected to the three electrodes. Further, Drake discloses “The ECG device 102 can include a signal processor 116, which can be configured to perform one or more signal processing operations on the signals received from the right arm electrode 108, from the left arm electrode 110, and from the left leg electrode 112” (see paragraph [0025]). Therefore, Drake receives three signals. Also, Drake is not clear about whether three signals are received simultaneously or sequentially. Drake also discloses “Various embodiments disclosed herein can relate to a handheld electrocardiogram simultaneous acquisition of six leads.” (see paragraph [0019]), where Drake uses the word “simultaneous” incorrectly, inappropriately and indefinitely. The structure of the device of Drake can be considered to be similar to that of the device of Thomson. In Drake, three electrodes are disposed on one side of the apparatus. Therefore, as with Thomson et al., it is difficult to bring three electrodes into contact with both hands and the body simultaneously.

The device of Saldivar (WO 2017/066040, 2017) uses a lead selection stage 250 to connect three electrodes to one amplifier 210. In addition, the device of Saldivar performs six measurements sequentially to obtain six leads. In other words, the device of Saldivar does not measure multiple leads simultaneously. The device of Saldivar also directly measures three augmented limb leads sequentially.

### SUMMARY OF INVENTION

### Problems to be Solved by Invention

Therefore, the present invention has been made in view of the above problems, and it is an object of the present invention to provide an electrocardiogram apparatus having three electrodes and two amplifiers associated with two limb leads to measure the two limb leads simultaneously with one electrocardiogram apparatus. It is medically very important to measure two limb leads simultaneously. This is because it is more time consuming and inconvenient to measure two leads sequentially. More importantly, two limb leads measured at different times may not correlate with each other and may cause confusion in detailed arrhythmia discrimination. The electrocardiogram apparatus according to the present invention includes a plate-shaped electrocardiogram apparatus having two dry electrodes separated from each other on one surface and one dry electrode disposed on the other surface for user convenience. It is another object of the present invention to provide a method for removing power line interference in order not to use a DRL electrode. It is an object of the present invention to disclose a convenient electrocardiogram measurement method bringing two electrodes into contact with two hands and one electrode into the body and an electrocardiogram measurement apparatus having a structure proper thereto.

### Means for Solving Problems

The appearance, operation principle, configuration, and usage of the electrocardiogram apparatus according to the present invention for solving the above problems are as follows. The present invention solves the above problems through systematic and analytical circuit design and software production.

In accordance with one aspect of the present invention, provided is an electrocardiogram measurement apparatus comprising:
a first electrode and a second electrode configured to receive two electrocardiogram voltages of a body part in contact therewith, respectively;
two amplifiers configured to receive the two electrocardiogram voltages from the first electrode and the second electrode, respectively;
one electrode driver configured to output a driving voltage;
a third electrode configured to receive the output of the electrode driver and transmit the output of the electrode driver to the body part in contact therewith;
an AD converter connected to an output terminal of each of the two amplifiers to convert output signals of the two amplifiers into two digital signals;
a microcontroller configured to receive the two digital signals of the AD converter; and
a communication means configured to transmit the two digital signals.

The microcontroller is supplied with battery power.

The microcontroller controls the AD converter and the communication means.

The two amplifiers each receive and amplify one electrocardiogram voltage simultaneously, and an output impedance of the electrode driver is less than an input impedance of each of the two amplifiers.

FIG. 1 shows an electrocardiogram measurement apparatus 100 according to the present invention. The electrocardiogram measurement apparatus 100 includes three electrodes 111, 112, and 113 on the surface thereof. Two electrodes 111 and 112 spaced apart from each other by a predetermined distance are installed on one surface of the electrocardiogram measurement apparatus 100, and one electrode 113 is installed on the other surface.

FIG. 2 illustrates a method for a user to measure an electrocardiogram in a 6-channel mode using the electrocardiogram measurement apparatus 100 according to the present invention. The user makes contact with the electrodes 111 and 112 provided on one surface of the electrocardiogram measurement apparatus 100 with both hands, and brings the electrode 113 provided on the other surface into contact with the left lower abdomen (or left leg) of the user. When the three electrodes are brought into contact with the user's body in this way, two limb leads can be measured, and four leads can be additionally calculated and obtained as described below. The measurement method of FIG. 2 is provided by the present invention to obtain a 6-channel electrocardiogram most conveniently. In addition, the present invention provides an apparatus most suitable for the measurement method of FIG. 2. The principle of the measurement method is as follows.

A traditional 12-lead ECG is disclosed in, for example, [ANSI/AAMI/IEC 60601-2-25:2011, Medical electrical equipment-part 2-25: Particular requirements for the basic safety and essential performance of electrocardiogram the traditional 12-lead ECG, three limb leads are defined as follows: lead I=LA-RA; lead II=LL-RA; lead III=LL-LA. In these equations, RA, LA, and LL denote the voltages of the right arm, left arm, and left leg, or body parts close to these limbs, respectively. Conventionally, in order to remove power line interference, a right leg (DRL) electrode is used. From the relationships above, one limb lead can be obtained from the other two limb leads. For example, lead III=lead II-lead I. Three augmented limb leads are defined as follows: aVR=RA−(LA+LL)/2; aVL=LA−(RA+LL)/2; aVF=LL−(RA+LA)/2. Therefore, the three augmented limb leads can be obtained from two limb leads. For example, aVR=−(I+II)/2. Therefore, when two limb leads are measured, the remaining four leads can be calculated and obtained. Accordingly, the present invention discloses an apparatus for simultaneously measuring two leads using three electrodes and two amplifiers to provide six leads. Here, one amplifier means that one signal is amplified. In an actual configuration, one amplifier may be configured as a set of multiple cascaded amplification stages or active filters. A standard 12-lead electrocardiogram consist of the six leads and six precordial leads from V1 to V6.

Modified chest leads (MCLs) are similar to the precordial leads and are medically very useful. In the principle of the present invention, the voltage of one electrode that is not connected to any amplifier among the three electrodes is substantially equal to the circuit common in the signal frequency band, as will be described later. Accordingly, the electrocardiogram measurement apparatus 100 according to the present invention is suitable for measuring one MCL among six MCLs from MCL1 to MCL6. This is because each MCL is a voltage at the position of the corresponding precordial lead referenced on the voltage of a body part to which the left hand is connected.

FIG. 3 illustrates a method for a user to measure MCL1 using the electrocardiogram apparatus according to the present invention in an MCL mode. For example, in order to measure MCL1 using the electrocardiogram apparatus according to the present invention, the user contacts the electrodes 111 and 112 provided on one surface of the electrocardiogram measurement apparatus 100 with both hands and brings the electrode 113 provided on the other surface into contact with the MCL position (for example, the V1 position in measuring MCL1), as shown in FIG. 3. In the present invention, in order for the user to measure MCLn, the user needs to bring the electrode 113 into contact with the MCLn position, that is, the Vn position on the user's body.

Hereinafter, an embodiment of the electrocardiogram measurement apparatus according to the present invention will be described with reference to FIGS. 4 and 5. FIG. 4 shows an electrical equivalent circuit model for explaining principles and embodiments of removing power line interference by the electrocardiogram measurement apparatus according to the present invention. FIG. 5 shows an electrical equivalent circuit model of an embodiment in which the electrocardiogram measurement apparatus according to the present invention simultaneously measures two channels of an electrocardiogram using two single-ended input amplifiers and one electrode driver.

In FIG. 4, a current source 450 is used to model power line interference. In addition, in FIG. 4, a human body is denoted by 430 and modeled by three electrode resistors 431, 432, and 433 connected to each other at one point. In FIG. 5, one electrocardiogram signal is modeled as one voltage source (461 or 462) existing between two electrode resistors. Since three electrodes are used in the present invention, in FIG. 5, it is modelled such that there are two ECG voltage sources 461 and 462 on the human body. This is because though there are three electrocardiogram voltages on the three electrodes (because the number of cases of selecting two of the three electrodes is 3), but only two electrocardiogram voltages are independent. The modeling for power line interference in FIG. 4 and the electrocardiogram signal in FIG. 5 is in a simplified form. However, the above models are suitable to clarify issues to be addressed. In addition, the above models clearly suggest what should be devised in the present invention. In addition, the present invention can be easily understood from the above models. The present invention has been devised based on the above models. Since the conventional arts do not use the above models, the conventional arts fail to accurately present a solution to the issues.

The present invention can be presented in various embodiments, as will be described later. However, the various embodiments of the present invention are commonly based on the following principle of the present invention. The principle of the present invention is devised for the present invention. The present invention differs from the conventional arts in that it does not use a DRL electrode compared to the DRL method used in the conventional arts.

A challenge that has not been overcome by a conventional electrocardiogram measurement apparatus that does not use a DRL electrode and is required to be overcome is to remove or reduce power line interference. Power line interference in the electrocardiogram measurement apparatus is caused by a current source having a substantially infinite output impedance due to a significantly high output impedance as shown in FIG. 4 (in FIG. 4, the power line interference current source is indicated by 450.). Accordingly, in order to remove the power line interference, it is necessary to minimize the impedance looking into the human body from the power line interference current source. The impedance looking into the human body from the power line interference current source is the sum of the impedance of the human body and the impedance of the electrocardiogram measurement apparatus. As a result, it is necessary to minimize the impedance of the electrocardiogram measurement apparatus looking into through the three electrodes. There exists an impedance called an electrode impedance or electrode resistance between each electrode used to measure the electrocardiogram and the human body (431, 432, and 433 in FIG. 4). Accordingly, in order to minimize the effect of the electrode impedance when measuring an electrocardiogram voltage, the electrocardiogram measurement apparatus should have a high impedance. Accordingly, the electrocardiogram measurement apparatus should satisfy two opposing conditions that a low impedance should be provided to remove power line interference and a high impedance should be provided to measure an electrocardiogram voltage.

A method that can be considered to satisfy the two opposing conditions, for example, when three electrodes are used, is to connect three large resistors to the three electrodes, respectively, combine the opposite ends of the three resistors at one point, and provide negative feedback of the common mode signals of the three electrodes to the one point at which the three resistors are combined. However, this method is practically difficult to use. This is because the impedance of the power line interference current source is large and thus the magnitude of the power line interference current will not decrease. Accordingly, in this case, the power line interference voltage induced in the three resistors is still quite large or the amplifiers may be saturated. In addition, since the magnitude of the power line interference current is not reduced and the impedances of the respective electrodes may be different, a different power line interference voltage is induced at a high level in each electrode. Accordingly, even if a differential amplifier is used, it is difficult to remove the power line interference induced in each electrode. This is the difficulty of the conventional arts.

Therefore, in the present invention, the power line interference current is concentrated and flows through only one of the electrodes installed in the electrocardiogram measurement apparatus. To this end, while three electrodes are connected to the human body, the impedance that the power line interference current source looks into the electrocardiogram measurement apparatus through the one electrode is minimizes. Thereby, the power line interference voltage (indicated by 440 ν_{body} in FIG. 4) induced in the human body by the power line interference current source is minimized. Since the power line interference voltage induced in the human body is minimized, the input impedances seen through the other electrodes of the electrocardiogram measurement apparatus may be increased, and the electrocardiogram voltage may be accurately measured. At this point, what is important is that the one electrode through which the power line interference current flows should not be used for measurement because a high power line interference voltage is induced at the one electrode. Accordingly, in the present invention, when three electrodes are used, two electrodes and two amplifiers to receive electrocardiogram signals from the two electrodes are used for measurement. In particular, it should be noted that two differential amplifiers cannot be used in the electrocardiogram measurement apparatus employing three electrodes because only two electrodes should be used for measurement. It should also be noted that, when negative feedback is used, if negative feedback is provided in all frequency bands then the electrocardiogram signals appear at the electrode and mixed with the power line interference voltage, and therefore negative feedback should be provided only at the power line interference frequency. Hereinafter, the present invention will be described in detail with reference to the drawings.

FIG. 4 and the subsequent drawings show only a part of the electrocardiogram measurement apparatus 100 according to the present invention for simplicity. In FIG. 4, the electrocardiogram measurement apparatus 100 according to the present invention comprises three electrodes 111, 112, and 113, two amplifiers 411 and 412, and one electrode driver (specifically, a band pass filter) 413. In FIGS. 4 and 5, the two amplifiers 411 and 412 employed in the present invention are not differential amplifiers, but are single-ended input amplifiers.

An important feature of the embodiment of the present invention shown in FIG. 4 is that the electrocardiogram measurement apparatus 100 comprises an electrode driver 413 represented as a band pass filter. That is, in FIG. 4 and the like, the electrode driver 413 may have a frequency characteristic of band pass. Accordingly, in the present invention, the electrode driver 413 may be described as a band pass filter. The input of the electrode driver 413 is connected to one electrode 112. The output of the electrode driver 413 drives the electrode 113 through the resistor 423 (it is fed back to the electrode 113). The resonance frequency or peak frequency of the electrode driver, that is, the band pass filter 413, is the same as the frequency of power line interference. In addition, the band pass filter 413 has a large Q. In FIG. 4, the input impedance of the band pass filter 413 is considerably large and the output impedance thereof is considerably small. The element value of the resistor 423 is represented by Ro. In the present invention, for simplicity, the resistor 423 is regarded as the output impedance of the electrode driver 413.

In the present invention, two of the three electrodes are connected to the circuit common of the analog circuit with the resistors 421 and 422, which have values Ri. The resistors 421 and 422 are regarded as input impedances of the amplifiers 411 and 412.

In FIG. 4, 430 is a model of a human body. There is a contact resistance, commonly called electrode impedance, between the human body and an electrode. In FIG. 4, the electrode impedances (electrode resistances) present between the human body 430 and the three electrodes 111, 112, and 113 are represented by resistors 431, 432, and 433, respectively. The element values of the electrode resistors 431, 432, and 433 are indicated by Rₑ₁, Rₑ₂, and Rₑ₃, respectively.

In FIG. 4, 450 is a power line interference current source for modeling power line interference. Current iₙ of the power line interference current source 450 flows to the circuit common of the electrocardiogram apparatus 100 according to the present invention through the human body 430 and the three electrodes 111, 112, and 113. When the power line interference currents flowing through the three electrodes 111, 112, and 113 are represented by iₙ₁, iₙ₂, and iₙ₃, the following equation is established according to Kirchhoff's current law.

$$\begin{matrix}Equation 1 & \\ {i}_{n} = {i}_{n 1} + {i}_{n 2} + {i}_{n 3} & ( 1 )\end{matrix}$$

For circuit analysis, power line interference induced in the human body 430 is denoted by ν_{body}. In FIG. 4, νₙ₁, νₙ₂, and νₙ₃ represent power line interference voltages at the electrodes 111, 112, and 113, respectively. In Equation 1, each current is given as follows.

$$\begin{matrix}Equation 2 & \\ {i}_{n 1} = \frac{{v}_{body}}{{R}_{i} + {R}_{e 1}} & ( 2 )\end{matrix}$$

$$\begin{matrix}Equation 3 & \\ {i}_{n 2} = \frac{{v}_{body}}{{R}_{i} + {R}_{e 2}} & ( 3 )\end{matrix}$$

$$\begin{matrix}Equation 4 & \\ {i}_{n 3} = \frac{{v}_{body} + {v}_{n 2} H ( f )}{{R}_{o} + {R}_{e 3}} & ( 4 )\end{matrix}$$

$$Here ,$$

$$\begin{matrix}Equation 5 & \\ {v}_{n 2} = \frac{{R}_{i}}{{R}_{i} + {R}_{e 2}} {v}_{body} & ( 5 )\end{matrix}$$

Here, the transfer function of the band pass filter 413 is denoted by −H(f). Using the equations above, the following equation is obtained.

$$\begin{matrix}Equation 6 & \\ {i}_{n} = \frac{{v}_{body}}{{R}_{i} + {R}_{e 1}} + \frac{{v}_{body}}{{R}_{i} + {R}_{e 2}} + \frac{{v}_{body}}{{R}_{o} + {R}_{e 3}} \frac{{R}_{i}}{{R}_{i} + {R}_{e 2}} H ( f ) , + \frac{{v}_{body}}{{R}_{o} + {R}_{e 3}} & ( 6 )\end{matrix}$$

In the present invention, the element values of the circuit of FIG. 4 are used so that the following approximations are possible (Equations 7 and 8). Equations 7 and 8 are important components of the present invention.

$$\begin{matrix}Equation 7 & \\ {R}_{i} ≫ {R}_{e 1} , {R}_{e 2} , or {R}_{e 3} & ( 7 )\end{matrix}$$

$$\begin{matrix}Equation 8 & \\ {R}_{i} ≫ {R}_{o} & ( 8 )\end{matrix}$$

Then, the following approximation is established.

$$\begin{matrix}Equation 9 & \\ {i}_{n} \approx \frac{{v}_{b}}{{R}_{o} + {R}_{e 3}} ( 1 + H ( f ) ) & ( 9 )\end{matrix}$$

The following equation is obtained from Equation 9.

$$\begin{matrix}Equation 10 & \\ {v}_{body} \approx ( {R}_{o} + {R}_{e 3} ) \frac{{i}_{n}}{( 1 + H ( f ) )} & ( 10 )\end{matrix}$$

In Equation 10, if there is no feedback, that is, H(f)=0, the following equation is established.

$$\begin{matrix}Equation 11 & \\ {v}_{body} \approx ( {R}_{o} + {R}_{e 3} ) {i}_{n} if H ( f ) ) = 0. & ( 11 )\end{matrix}$$

By comparing Equation 10 and Equation 11, it can be seen that the present invention reduces the influence of power line interference current iₙ to the amount of feedback, or (1+H(f)). Therefore, if the magnitude of the gain at the resonance frequency of the band pass filter satisfies |H(f₀)|»1, ν_{body}≈0. Thus, the principle of removing power line interference in the present invention has been proved.

Using Equations 2 and 10, the following can be confirmed.

$$\begin{matrix}Equation 12 & \\ {v}_{n 1} \approx \frac{{R}_{i}}{{R}_{i} + {R}_{e 1}} ( {R}_{o} + {R}_{e 3} ) \frac{{i}_{n}}{1 + H ( f )} , \approx ( {R}_{o} + {R}_{e 3} ) \frac{{i}_{n}}{1 + H ( f )} , \approx {v}_{body} & ( 12 )\end{matrix}$$

Now the following result is obtained for νₙ₃. From the above results, ν_{body}≈0 and iₙ₃? aaiₙ can be used.

$$\begin{matrix}Equation 13 & \\ {v}_{n 3} \approx {v}_{body} - {i}_{n 3} {R}_{e 3} \approx - {i}_{n} {R}_{e 3} & ( 13 )\end{matrix}$$

The following can be derived from Equations 12 and 13.

$$\begin{matrix}Equation 14 & \\ \vert {v}_{n 3} \vert ≫ \vert {v}_{n 1} \vert & ( 14 )\end{matrix}$$

This means that, if |H(f)| is large, as a result of feedback, almost all power line interference current flows through the electrode (the electrode 113 in FIG. 4) to which feedback is provided, and therefore the electrode to which feedback is provided is contaminated by power line interference while the electrodes (the electrodes 111 and 112 in FIG. 4) to which feedback is not provided are hardly influenced by power line interference. This in turn means that only the electrodes to which feedback is not provided should be used for electrocardiogram measurement and the electrode to which feedback is provided should not be used for the measurement. Accordingly, the effect of power line interference cannot be eliminated using a differential amplifier whose input is connected to the electrodes 111 and 113 or a differential amplifier whose input is connected to the electrodes 112 and 113. This is one of the important issues of the conventional arts.

Hereinafter, description will be given of the principle of obtaining two electrocardiogram channel signals using three electrodes according to the present invention. FIG. 5 shows an electrical equivalent circuit given when an electrocardiogram is measured using the electrocardiogram apparatus according to the present invention.

In FIG. 5, ν₁, ν₂, and ν₃ represent the electrocardiogram signal voltages of the electrodes 111, 112, and 113, respectively. Voltage ν₂ of the electrode 112 obtained by analyzing this equivalent circuit based on the principle of superposition is given as follows.

$$\begin{matrix}Equation 15 & \\ & ( 15 )\end{matrix}$$

$${v}_{2} = - {v}_{a} \frac{( {R}_{o} + {R}_{e 3} ) ∥ ( {R}_{i} + {R}_{e 2} )}{( {R}_{i} + {R}_{e 1} ) + ( {R}_{o} + {R}_{e 3} ) ∥ ( {R}_{i} + {R}_{e 2} )} \frac{{R}_{i}}{( {R}_{i} + {R}_{e 2} )} , + {v}_{b} \frac{( {R}_{i} + {R}_{e 1} ) ∥ ( {R}_{i} + {R}_{e 2} )}{( {R}_{i} + {R}_{e 1} ) ∥ ( {R}_{i} + {R}_{e 2} ) + ( {R}_{i} + {R}_{e 1} )} \frac{{R}_{i}}{( {R}_{i} + {R}_{e 2} )} , - {v}_{2} H ( f ) \frac{( {R}_{i} + {R}_{e 1} ) ∥ ( {R}_{i} + {R}_{e 2} )}{( {R}_{o} + {R}_{e 3} ) + ( {R}_{i} + {R}_{e 1} ) ∥ ( {R}_{i} + {R}_{e 2} )} \frac{{R}_{i}}{( {R}_{i} + {R}_{e 2} )} .$$

In Equation 15, the symbol ∥ represents the value of parallel resistance. As in the previous equations, the conditions of Equations 7 and 8 can be assumed. In this case, voltage ν₂ is approximated as follows.

$$\begin{matrix}Equation 16 & \\ {v}_{2} \approx - {v}_{a} \frac{( {R}_{o} + {R}_{e 3} )}{( {R}_{i} + {R}_{e 3} )} \frac{{R}_{i}}{( {R}_{i} + {R}_{e 2} )} + {v}_{b} - {v}_{2} H ( f ) \approx {v}_{b} - {v}_{2} H ( f ) & ( 16 )\end{matrix}$$

Accordingly, under the conditions of Equations 7 and 8, voltage ν₂ is given as follows.

$$\begin{matrix}Equation 17 & \\ {v}_{2} \approx {v}_{b} \frac{{1}_{}}{1 + H ( f )} & ( 17 )\end{matrix}$$

From the above equation, it can be seen that if |(H(f₀)|«1, v₂≈v_{b} in the signal band.

FIG. 6 shows a frequency response of the band pass filter used in the electrocardiogram measurement apparatus according to the present invention. In FIG. 6, the resonance frequency of the band pass filter is 60 Hz, the gain at the resonance frequency is 20, and Q=120. FIG. 7 shows that when the band pass filter of FIG. 6 is used, ν_{b} may be obtained with an accuracy of 98% at a frequency less than or equal to 40 Hz.

Similarly, voltage ν₁ of the electrode 1 is obtained as follows.

$$\begin{matrix}Equation 18 & \\ & ( 18 )\end{matrix}$$

$${v}_{1} = + {v}_{a} \frac{{R}_{i}}{( {R}_{i} + {R}_{e 1} ) + ( {R}_{o} + {R}_{e 3} ) ∥ ( {R}_{i} + {R}_{e 2} )} , + {v}_{b} \frac{( {R}_{i} + {R}_{e 1} ) ∥ ( {R}_{i} + {R}_{e 2} )}{( {R}_{o} + {R}_{e 3} ) + ( {R}_{i} + {R}_{e 1} ) ∥ ( {R}_{i} + {R}_{e 2} )} \frac{{R}_{i}}{( {R}_{i} + {R}_{e 1} )} , - {v}_{2} H ( f ) \frac{( {R}_{i} + {R}_{e 1} ) ∥ ( {R}_{i} + {R}_{e 2} )}{( {R}_{o} + {R}_{e 3} ) + ( {R}_{i} + {R}_{e 1} ) ∥ ( {R}_{i} + {R}_{e 2} )} \frac{{R}_{i}}{( {R}_{i} + {R}_{e 2} )} .$$

When the conditions of Equations 7 and 8 are used, voltage ν₁ is approximated as follows.

$$Equation 19$$

$${v}_{1} \approx + {v}_{a} + {v}_{b} - {v}_{2} H ( f ) \approx {v}_{a} + {v}_{2}$$

The equation above is obtained using Equation 16. Equation 20 below is obtained from the equation above, and Va may be obtained by this equation. It can be seen from Equation 20 that Va can be obtained without the influence of the band pass filter.

$$\begin{matrix}Equation 20 & \\ {v}_{1} - {v}_{2} \approx + {v}_{a} & ( 20 )\end{matrix}$$

1 1

Thus, the principle of obtaining signals of two electrocardiogram channels using two single-ended amplifiers according to the present invention has been described.

FIG. 8 shows an electrical equivalent circuit model for explaining the principle and embodiment of removing power line interference by the electrocardiogram measurement apparatus according to the present invention using common mode signal of two electrodes. Of course, even when the common mode signal is used, the power line interference current is concentrated and flows through an electrode 832 to which the output of an electrode driver 813 is fed back, and a power line interference voltage is present in the electrode 832. FIG. 9 shows an electrical equivalent circuit model of an embodiment of simultaneously measuring two channels of an electrocardiogram using one differential amplifier 811 and one single-ended input amplifier 812 while removing power line interference using the method of FIG. 8, that is, adopting common mode signal. As in the previous case where two single-ended input amplifiers are used, two electrocardiogram voltages may be obtained.

For simplicity, the circuit analysis of FIGS. 8 and 9 is omitted. In the embodiment of FIGS. 8 and 9, one electrode driver, that is, the band pass filter 813, applies a driving voltage to a human body part in contact with the electrode 112 through the output impedance Ro and the electrode 112, as in the embodiment of FIGS. 4 and 5. That is, the electrode 112 is not connected to an amplifier for measuring the electrocardiogram voltage, but is connected to the electrode driver 813 through the output impedance 823. That is, the electrode 112 is not used in measuring the electrocardiogram voltage. When the peak value of the band pass filter is large, the transfer function H(f) of the band pass filter may be corrected or compensated for in order to realize |H(f)|«1 in the signal band.

While one band pass filter 813 is used as one electrode driver in FIG. 8, one constant voltage source 1013 is used as one electrode driver in FIG. 10. The constant voltage source 1013 applies a driving voltage to a human body part in contact with the electrode 112 through a resistor 1023 having a small resistance Rₒ and the electrode 112. Most of the power line interference current flows through the electrode 112. In order to further concentrate the power line interference current, the output impedance 1023 of the electrode driver 1013 may be reduced. This method is less effective in removing power line interference than the previous methods using a band pass filter. Even in the embodiment of FIG. 10, two electrocardiogram voltages are simultaneously amplified using two amplifiers connected to two electrodes except for the electrode in which power line interference current is concentrated. In the embodiment of FIG. 10, one differential amplifier 1011 and one single-ended input amplifier 1012 are used. The output of the single-ended input amplifier 1012 may include weak power line interference and a band pass filter 1033 may be used to further reduce power line interference.

In FIGS. 5, 9 and 10, it is important to drive one electrode using an electrode driver having a small output impedance in order to reduce the power line interference. The output of the electrode driver is transmitted, through the electrode, to a human body part that is in contact with the electrode. Once the power line noise is reduced, two single-ended input amplifiers may be used or one single-ended input amplifier and one differential amplifier to amplify the two electrocardiogram voltages received from the two electrodes.

The principle of the present invention is summarized as follows. The condition that the input impedance the power line interference current source looks into the electrocardiogram measurement apparatus should be low is satisfied by reducing the output impedance of the electrode driver connected to one electrode, and the condition that the input impedances the electrocardiogram signal voltages are looking into the electrocardiogram measurement apparatus should be high is satisfied by increasing the input impedances seen through the other two electrodes. Thereby, the electrocardiogram measurement apparatus according to the present invention may accurately measure the electrocardiogram signal voltage while reducing power line interference. Accordingly, the output impedance of the electrode driver of the electrocardiogram measurement apparatus according to the present invention is less than the input impedance of each of the two amplifiers.

Description has been given above regarding an embodiment in which power line interference is removed by applying the output of one electrode driver to one electrode, and two electrocardiogram voltages are measured simultaneously using two amplifiers of a large input impedance that receive two electrocardiogram voltages from two electrodes.

### Advantageous Effects

The electrocardiogram measurement apparatus according to the present invention provides six electrocardiogram leads obtained simultaneously using the smallest number of electrodes (specifically, three electrodes). When the electrocardiogram measurement apparatus according to the present invention is used in the MCL mode, one limb lead (specifically, Lead I) and one MCL may be measured.

Since the portable electrocardiogram measurement apparatus according to the present invention has a size of one credit card, it is convenient to carry the apparatus, and multiple electrocardiograms may be obtained most conveniently regardless of time and place. In addition, since the electrocardiogram measurement apparatus according to the present invention is capable of wirelessly communicating with a smartphone, the electrocardiogram measurement apparatus may be conveniently used without substantial limitation on the distance between the electrocardiogram measurement apparatus and the smartphone.

In addition, when the electrocardiogram measurement apparatus according to the present invention is not in use, all circuits except the current detectors are turned off and only the microcontroller enters a sleep mode. When the electrocardiogram measurement apparatus is used, only necessary circuits are supplied with power, and the microcontroller enters an activation mode. Therefore, consumption of power of the battery embedded in the electrocardiogram measurement apparatus may be reduced to the maximum degree.

In addition, the electrocardiogram measurement apparatus according to the present invention does not include a mechanical power switch or a selection switch. Accordingly, the measurement apparatus may be designed to be compact and slim, and may not lead to unnecessary troublesome use of a switch by the user, failure and finite service life of the switch, or an increase in manufacturing cost.

Further, since the electrocardiogram measurement apparatus according to the present invention does not include a display such as an LCD, there may no possibility of failure and deterioration of the display, and the apparatus may not lead to an increase in manufacturing cost, and may be manufactured in a compact size and convenient to carry.

### BRIEF DESCRIPTION OF DRAWINGS

FIG. 1 is a perspective view of an electrocardiogram measurement apparatus having three electrodes according to the present invention.

FIG. 2 illustrates a method for measuring an electrocardiogram in a 6-channel mode using the electrocardiogram measurement apparatus 100 according to the present invention.

FIG. 3 illustrates a method for measuring an electrocardiogram in an MCL mode using the electrocardiogram apparatus according to the present invention.

FIG. 4 shows an electrical equivalent circuit model for explaining the principle and embodiment of removing power line interference by the electrocardiogram measurement apparatus according to the present invention.

FIG. 5 shows an electrical equivalent circuit model of an embodiment in which the electrocardiogram measurement apparatus according to the present invention simultaneously measures two channels of an electrocardiogram using two single-ended input amplifiers and one band pass filter (electrode driver).

FIG. 6 shows a frequency response of the band pass filter used as an electrode driver in the electrocardiogram measurement apparatus according to the present invention.

FIG. 7 shows a frequency response of one signal channel when a band pass filter is used as an electrode driver in the electrocardiogram measurement apparatus according to the present invention.

FIG. 8 shows an electrical equivalent circuit model for explaining the principle and embodiment of removing power line interference by the electrocardiogram measurement apparatus according to the present invention using the common mode signal.

FIG. 9 shows an electrical equivalent circuit model of an embodiment of simultaneously measuring two channels of an electrocardiogram by the electrocardiogram measurement apparatus according to the present invention, using one differential amplifier, one single-ended input amplifier and one band pass filter (electrode driver) in removing power line interference using the common mode signal.

FIG. 10 is another embodiment of simultaneously measuring two channels of an electrocardiogram by the electrocardiogram measurement apparatus according to the present invention using one differential amplifier, one single-ended input amplifier, and one constant voltage generator (electrode driver).

FIG. 11 is a block diagram of a circuit embedded in the electrocardiogram measurement apparatus according to the present invention.

FIG. 12 is an operation flowchart of the electrocardiogram measurement apparatus according to the present invention.

FIG. 13 shows an initial screen of a smartphone when a smartphone app is executed to use the electrocardiogram measurement apparatus according to the present invention

FIG. 14 is a flowchart illustrating a smartphone app operated when the electrocardiogram measurement apparatus according to the present invention is used.

FIG. 15 shows an electrocardiogram measurement apparatus according to the present invention provided with a blood test strip insert port

FIG. 16 shows an example of implementing the electrocardiogram measurement apparatus according to the present invention in the form of a smart watch.

FIG. 17 shows an example of implementing the electrocardiogram measurement apparatus according to the present invention in the form of a ring.

FIGS. 18 and 19 show examples of the electrocardiogram measurement apparatus configured to be coupled to pants to measure an electrocardiogram according to the present invention.

FIGS. 20 and 21 show embodiments of the electrocardiogram measurement apparatus that can be coupled to the band of a watch using two or one slide guide that serves as two or one electrode according to the present invention.

FIG. 22 is a perspective view of an electrocardiogram measurement apparatus having four electrodes according to the present invention.

FIG. 48 illustrates subset embodiments of Embodiment F3, which include three electrodes, an electrode driver, and two amplifiers, wherein the electrode driver is an amplifier, an input of the electrode driver is connected to a right-hand electrode, and an output thereof is connected to a left-hand electrode.

FIG. 49 illustrates embodiments of Embodiment Group H, which include three electrodes, an electrode driver, and two single-ended input amplifiers, wherein the electrode driver is a band-pass filter or a constant voltage source, and an output of the electrode driver is connected to an electrode.

FIG. 50 illustrates subset embodiments of Embodiment 49A, which include three electrodes, an electrode driver, and two amplifiers, wherein the electrode driver is a band-pass filter, an input of the electrode driver is connected to a left-hand electrode, and an output thereof is connected to a right-hand electrode.

FIG. 51 illustrates subset embodiments of Embodiment 49H, which include three electrodes, an electrode driver, and two amplifiers, wherein the electrode driver is a constant voltage source, and an output of the constant voltage source is connected to a left-hand electrode.

FIG. 52 illustrates subset embodiments of Embodiment A1 of FIG. 27, which include four electrodes, an electrode driver, and two amplifiers, wherein the electrode driver is an amplifier, an input of the electrode driver is connected to a right-hand electrode, and an output thereof is connected to another right-hand electrode disposed adjacent to the right-hand electrode.

FIG. 53 illustrates embodiments of Embodiment Group G, which include three electrodes, an electrode driver, a differential amplifier, and a single-ended input amplifier, wherein the electrode driver is an amplifier, an input of the electrode driver is connected to a center point of a voltage divider, and an output thereof is connected to an electrode.

FIG. 54 illustrates subset embodiments of Embodiment Group C, which include four electrodes, an electrode driver, a differential amplifier, one single-ended input amplifier, and one differential amplifier, wherein the electrode driver is an amplifier, an input of the electrode driver is connected to a center point of a voltage divider, and an output thereof is connected to a fourth electrode.

FIG. 55 illustrates embodiments of Embodiment Group I, which include three electrodes, an electrode driver, a differential amplifier, and a single-ended input amplifier, wherein the electrode driver is a band-pass filter, an input of the band-pass filter is connected to a center point of a voltage divider, and an output thereof is connected to an electrode.

FIG. 56 illustrates embodiments of Embodiment Group J, which include three electrodes, an electrode driver, and two amplifiers, wherein the electrode driver is a constant voltage source, an output of the constant voltage source is connected to an electrode, a first amplifier is a single-ended input amplifier connected to a center point of a voltage divider, and a second amplifier is a differential amplifier connected across both ends of the voltage divider.

FIG. 57 is Table 8, which shows magnitudes of power-line interferences included in six limb-lead electrocardiogram signals obtained by measurement or calculation in an electrocardiogram measuring apparatus according to Embodiment F1 of FIG. 41.

FIG. 58 is Table 9, which shows magnitudes of power-line interferences included in six limb-lead electrocardiogram signals obtained by measurement or calculation in electrocardiogram measuring apparatuses according to Embodiment F3 of FIG. 41 and Embodiment 49H of FIG. 49.

### MODE FOR CARRYING OUT INVENTION

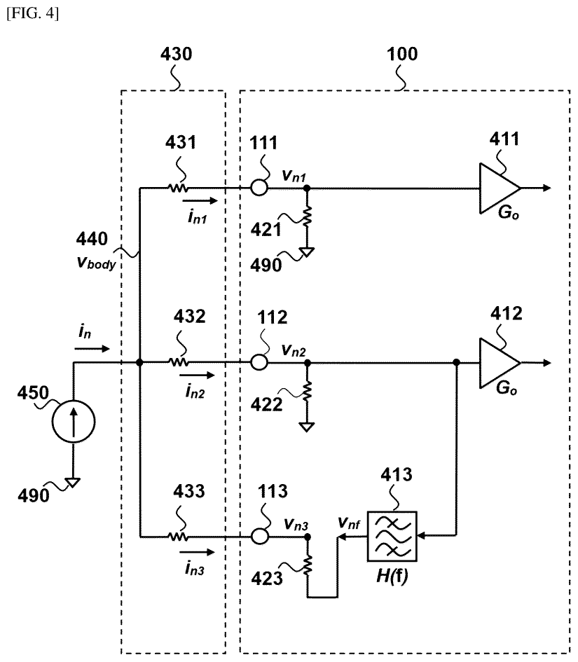

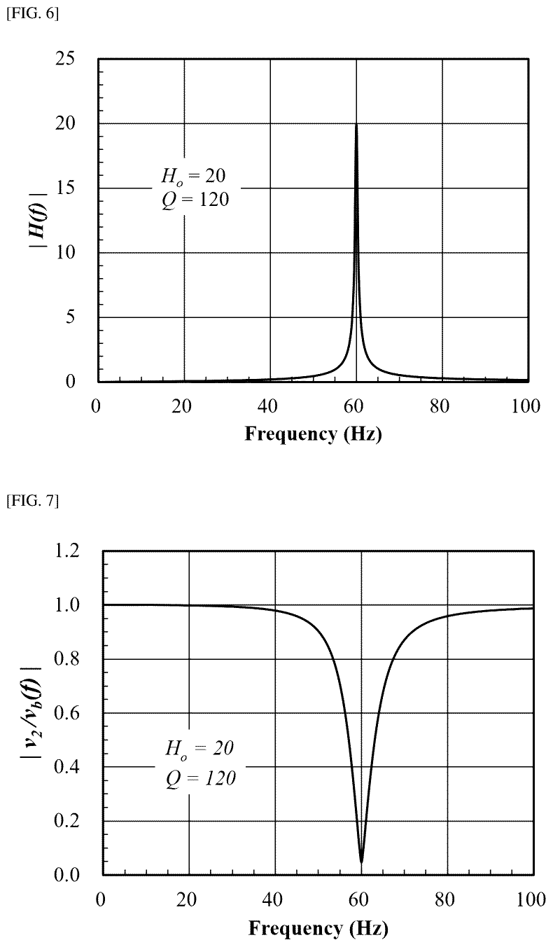

Hereinafter, an embodiment according to the present invention will be described with reference to the drawings. In this embodiment, an electrocardiogram (ECG) measurement apparatus is described as including three electrodes, but is not limited thereto. The electrocardiogram measurement apparatus may include three or more electrodes. An important embodiment of the present invention has been described above based on FIGS. 4 to 10 to explain the principle of the present invention.

The portable electrocardiogram measurement apparatus according to the present invention may be in the form of a credit card and have a thickness of 6 mm or less in order to enhance portability. Since the portable electrocardiogram measurement apparatus according to the present invention is portable, it uses a battery. When a CR2032 type battery is employed, the service life thereof may be about 2 years.

In addition, to make the portable electrocardiogram measurement apparatus compact, either a mechanical power switch or a selection switch may not be provided. In addition, to reduce power consumption, a display is not employed.

The portable electrocardiogram measurement apparatus according to the present invention may employ a current detector in order not to use a mechanical power switch or a selection switch. The current detector is always supplied with power required for operation and waits to generate an output signal when an event occurs. When a user brings multiple electrodes into contact with the body to measure an electrocardiogram, a loop of minute current that can flow through the human body is generated. Accordingly, when the body is electrically connected to the current detector, the current detector causes the minute current to flow through the body. Upon detecting the minute current, the current detector generates an output signal. When the portable electrocardiogram apparatus is not in use, only the current detector operates, and the other circuits are powered off, and the microcontroller waits in a sleep mode in order to increase the battery usage time. At this time, when an event of touching two electrodes by both hands occurs and the current detector generates an output signal, the microcontroller is activated to power on the electrocardiogram circuit to perform electrocardiogram measurement. The current detected by the current detector is supplied from the battery provided in the portable electrocardiogram measurement apparatus, and is a direct current.

The electrocardiogram measurement apparatus 100 according to the present invention may further include a function of measuring blood properties such as blood glucose level, ketone level, or international normalized ratio (INR). Accordingly, in this embodiment, the electrocardiogram measurement apparatus 100 will be described as an example for measuring an electrocardiogram and blood properties together. The blood glucose level or ketone level may be measured using an amperometric technique. The INR is a measure of the tendency to coagulate blood and may be measured for capillary blood using an electric impedance technique, the amperometric technique, a mechanical technique, or the like. One blood test strip insert port through which a blood test strip required for the blood property test can be inserted may be provided in the case of the electrocardiogram measurement apparatus according to the present invention.

In an embodiment of the electrocardiogram measurement apparatus 100 according to the present invention, a thermometer function may be included. A suitable type to include the thermometer function in the electrocardiogram measurement apparatus 100 according to the present invention is a contact type, and a suitable temperature sensor is a thermistor. In order to measure body temperature using the electrocardiogram measurement apparatus 100 including the thermometer function according to the present invention, a user brings a portion of the electrocardiogram measurement apparatus 100 to which the temperature sensor is attached into contact with the user's forehead or armpit. To accurately measure the body temperature, the temperature of the skin should not be changed by the portion of the electrocardiogram measurement apparatus 100 to which the temperature sensor is attached.

FIG. 11 is a block diagram of a circuit embedded in the electrocardiogram measurement apparatus according to the present invention. Although not shown in FIG. 11 for clarity of the invention, the electrocardiogram measurement apparatus according to the present invention may include a blood test circuit and a blood test strip insert port. The function and operation of each block in FIG. 11 are described below. When the user touches a pair of electrodes 111 and 112 with both hands, an electrocardiogram current detector 1140 allows minute current to flow through both hands and detects the minute current flowing through both hands. Then, the current detector 1140 generates a signal to change the microcontroller 1180 from a sleep mode to an active mode. Then, the microcontroller 1180 powers on the electrocardiogram measurement circuit 1160 and the AD converter 1170. The electrocardiogram measuring circuit 1160 amplifies two electrocardiogram signals through two amplifiers and generates two outputs. The AD converter 1170 receives the two outputs of the electrocardiogram measurement circuit 1160, and the outputs of the AD converter 1170 are transmitted to the smartphone 210 through the wireless communication means 1190 and the antenna 1192. Upon receiving data, the smartphone 210 displays multiple electrocardiogram waveforms. After the measurement for a certain duration, the microcontroller 1180 enters the sleep mode and waits for the next touch of both hands.

When the electrocardiogram measurement apparatus according to the present invention is brought into contact with both hands and the lower left abdomen, six leads can be displayed at a time. However, when it is inconvenient to bring the electrocardiogram measurement apparatus into contact with the lower left abdomen or only one lead is to be measured, the electrocardiogram measurement apparatus may automatically determine whether the user intends to measure only one lead or six leads. When the user touches the electrocardiogram measurement apparatus with only both hands to measure only one lead, only one current detector 1140 detects current. Then, only Lead I is displayed on the smartphone. When the user touches the electrocardiogram measurement apparatus with both hands and the lower left abdomen to measure six leads, both the current detector 1140 and the current detector 1150 detect currents. The six leads are then displayed on the smartphone. Each of the blocks shown in FIG. 11 may be implemented based on conventional technology using commercialized parts.

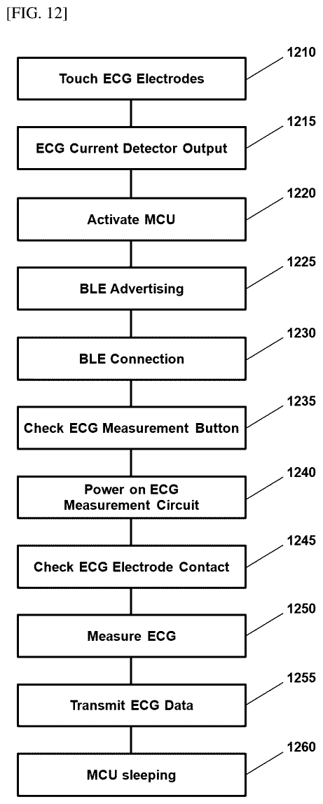

FIG. 12 is an operation flowchart of the electrocardiogram measurement apparatus 100 according to the present invention in measuring an electrocardiogram. In order to measure the electrocardiogram, a user touches the pair of electrodes 111 and 112 of the electrocardiogram measurement apparatus 100 with both hands (1210). Then, the current detector detects minute current flowing through the human body between both hands and generates an output signal (1215). The output signal activates the microcontroller 1180 by generating an interrupt of the microcontroller 1180 (1220). The activated microcontroller 1180 activates the wireless communication means 1190. Hereinafter, a case where the wireless communication means 1190 is a Bluetooth low energy device will be described. The wireless communication means 1190 of the electrocardiogram measurement apparatus 100 advertises as a Bluetooth low energy peripheral (1225). At this time, the smartphone that is performing scanning as a Bluetooth low energy central device discovers the electrocardiogram measurement apparatus 100 and attempts to connect thereto. At this time, when the electrocardiogram measurement apparatus 100 approves the connection, the smartphone and the electrocardiogram measurement apparatus 100 are Bluetooth low energy connected (1230). At this time, the electrocardiogram measurement apparatus 100 may check whether the user has touched the electrocardiogram measurement button of the smartphone to actually measure an electrocardiogram (1235).

Once it is confirmed that electrocardiogram measurement is requested, the microcontroller 1180 powers on the electrocardiogram measurement circuit 1160 (1240). This operation may be performed by connecting an output pin of the microcontroller 1180 to the electrocardiogram measurement circuit 1160 and setting the voltage of the output pin to High. Next, it is checked whether the pair of electrodes 111 and 112 are in touch with both hands, using the current detector (1245). This step is to determine when the microcontroller 1180 should start ECG measurement, that is, AD conversion. That is, this step is to check whether both hands continuously remain in contact with the electrodes 111 and 112.

After the above steps, the microcontroller 1180 starts the ECG measurement (1250). That is, the microcontroller 1180 performs AD conversion according to a preset AD conversion cycle and brings an AD conversion result. In the present invention, two electrocardiogram signals are measured. The measured ECG data is transmitted to the smartphone 210 (1255). When a preset measurement time of, for example, 30 seconds, elapses, the microcontroller 1180 enters the sleep mode (1260).

All circuits of FIG. 11 are driven by a battery embedded in the electrocardiogram measurement apparatus 100. In the example of FIG. 11, any of a mechanical power switch, a mechanical selection switch, and a display may not be provided. In FIG. 11, when the electrocardiogram measurement apparatus 100 does not perform measurement, the electrocardiogram current detector and the microcontroller 1180 each consume approximately 1 uA, and all the other blocks are completely powered off.

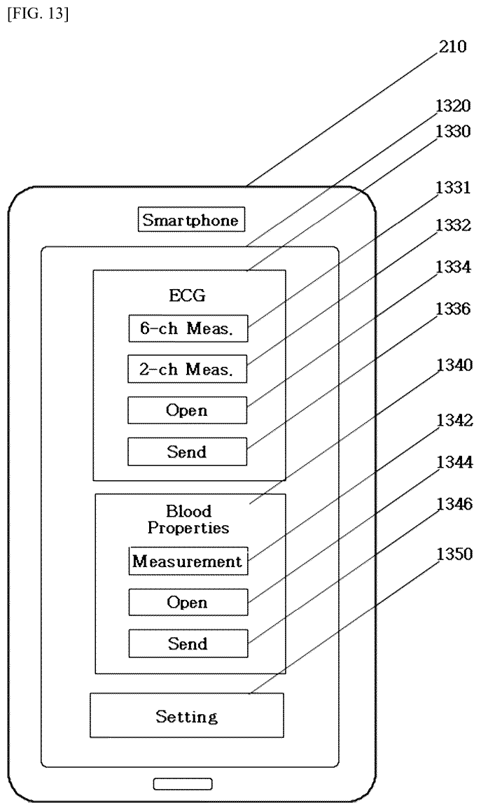

The electrocardiogram measurement apparatus 100 according to the present invention is used together with the smartphone 210. FIG. 13 shows an initial screen of a smartphone when a smartphone app according to the present invention is executed. When the smartphone app is executed, touch buttons 1331, 1332, 1334, 1336, 1342, 1344, 1346, and 1350 are displayed on the display 1320 of the smartphone 210. The buttons 1331, 1332, 1334, and 1336 related to the electrocardiogram are configured in an electrocardiogram box 1330. When the electrocardiogram measurement apparatus 100 according to the present invention includes a function of measuring blood properties, the buttons 1342, 1344 and 1346 related to blood properties are configured in a blood glucose box 1340. To measure an electrocardiogram, the user selects and touches one of the electrocardiogram measurement mode buttons 1331 and 1332 wanted. When the user is to measure the electrocardiogram in a 6-channel mode, the user touches the button 1331. When the user is to measure the electrocardiogram in an MCL mode, the user touches the button 1332. Then, when the user remains touching the pair of electrodes 111 and 112 of the electrocardiogram measurement apparatus 100 with both hands, the electrocardiogram measurement apparatus 100 measures the electrocardiogram as described above. The measured ECG data is displayed in the form of a chart on the smartphone display 1320 and is stored in the smartphone 210. The open button 1334 is touched to view, in a chart form, the ECG measurement data stored in the past. To send the stored data to a doctor or a hospital, the Send button 1336 is touched. The Setting button 1350 is touched when a user's name, date of birth, gender, address, etc. are to be recorded or when options are to be set.

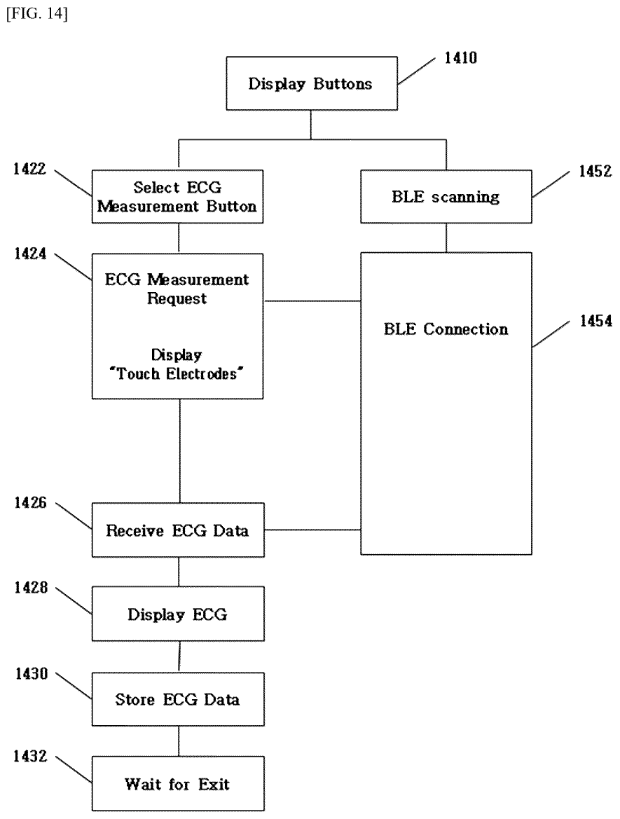

FIG. 14 is a flowchart illustrating a smartphone app according to the present invention. For simplicity, only the process of measuring an electrocardiogram will be described. As shown in FIG. 14, the flow operated in measuring the electrocardiogram is composed of two branches: a central branch 1422, 1424, 1426, 1428, 1430, 1432, and a Bluetooth low energy (BLE) branch 1452, 1454. When the app starts, various buttons appear on the smartphone display 1320 (1410), and then the BLE branch 1452 1454 for performing Bluetooth low energy communication is started. The user who wants to measure the electrocardiogram touches one of the ECG measurement buttons 1331 and 1332 (1422).

When the user touches one of the ECG measurement buttons 1331 or 1332 (1422), an ECG measurement request signal is sent to the BLE branch 1452, 1454 (1424). In addition, a message instructing the user to contact electrodes according to the ECG measurement mode is displayed on the smartphone display 1320 (1424). In the BLE branch 1452, 1454, an ECG measurement request signal is sent to the electrocardiogram measurement apparatus 100 (1454).

The electrocardiogram measurement apparatus 100 receiving the ECG measurement request signal performs the electrocardiogram measurement task described in FIG. 12 and transmits measured ECG data to the BLE branch 1452, 1454. The BLE branch 1452, 1454 transfers the ECG data received from the electrocardiogram measurement apparatus 100 to the central branch 1422, 1424, 1426, 1428, 1430, 1432. Then, the central branch 1422, 1424, 1426, 1428, 1430, 1432 receives the ECG data (1426). The received ECG data is displayed in a chart form on the smartphone display 1320 in the central branch 1422, 1424, 1426, 1428, 1430, 1432 (1428). When all the ECG measurements are completed, the measured ECG data is stored in a file format in a smartphone storage device (1430). While the measured ECG data is being displayed in the form of a chart on the smartphone display 1320, the smartphone app waits for the user to end the app by pressing the app exit button (1432).

According to the present invention, the user may be provided with desired results without undergoing abnormality in the number of cases of all possible operation sequences by using the electrocardiogram measurement apparatus 100, which is not provided with a mechanical switch, a selection switch, or a display, and a smartphone app simplified to use.

The present invention has been described in detail regarding a case where an electrocardiogram is measured using the single portable electrocardiogram measurement apparatus 100 and a smartphone app, but the electrocardiogram measurement apparatus 100 according to the present invention is not limited thereto. Various measurement items may be additionally measured.

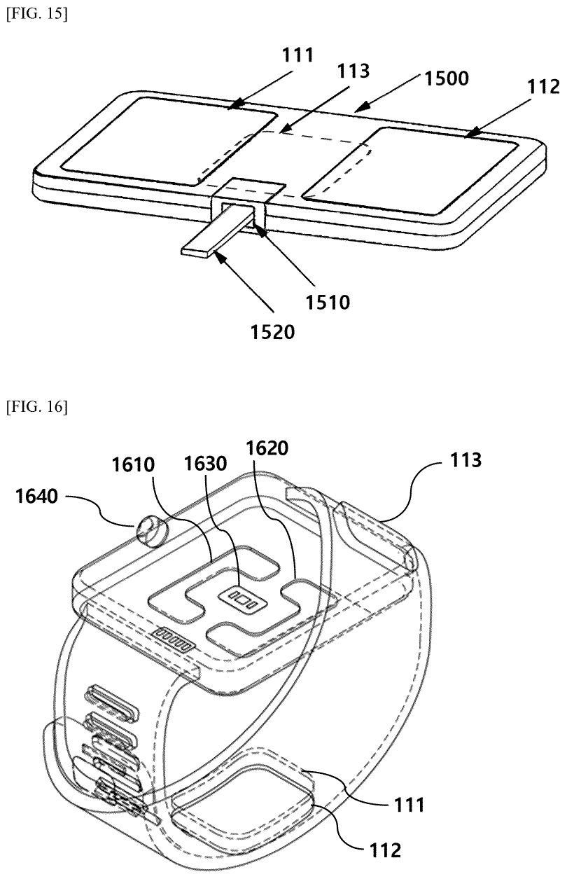

As described above, the electrocardiogram measurement apparatus 100 according to the present invention may further include a function of measuring blood properties. In this case, one embodiment of the electrocardiogram measurement apparatus 1500 to which the function of measuring blood properties is added according to the present invention includes a blood property test strip insert port 1510 through which a blood property test strip 1520 can be inserted, and one type thereof may be configured as shown in FIG. 15.

The electrocardiogram measurement apparatus 100 according to the present invention has been described as being implemented in a plate shape. However, the electrocardiogram measurement apparatus according to the present invention uses the minimum number of filters in principle and has a simple circuit configuration, and accordingly it can be manufactured in a compact size. Accordingly, the electrocardiogram measurement apparatus according to the present invention has a feature that the power consumption of the battery is low. Accordingly, the electrocardiogram measurement apparatus according to the present invention is suitable to be implemented as a watch or ring shape. Particularly, when the electrocardiogram measurement apparatus according to the present invention is implemented as a watch shape or a ring shape, it is suitable for a user to always wear and has an advantage that it can be used in conjunction with a photoplethysmograph (PPG).

The PPG uses LEDs to emit light to the skin and measure reflected or transmitted light. Recently, the PPG built in the smart watch can provide heart rate, heart rate variability (HRV), and breathing rate (BR). HRV provides a lot of information about personal health conditions. HRV is used for sleep analysis or stress analysis, and is also used to detect arrhythmias such as atrial fibrillation. Normally, HRV analysis is performed using ECG. However, recently, it has also been performed using PPG. The PPG included in a patient monitor used in hospitals measures oxygen saturation and generates an alarm when the oxygen saturation is low. Recently, a PPG signal is obtained using a camera installed in a smartphone, and the occurrence of an arrhythmia symptom may be detected using the signal. Accordingly, PPG installed on the watch or ring facilitates detection of occurrence of an arrhythmia symptom. Accordingly, when the PPG and the electrocardiogram measurement apparatus according to the present invention are installed together on a watch or ring, the PPG may generate an alarm signal upon detecting occurrence of arrhythmia symptoms, and the user who receives the alarm signal can measure the electrocardiogram using the electrocardiogram measurement apparatus according to the present invention.

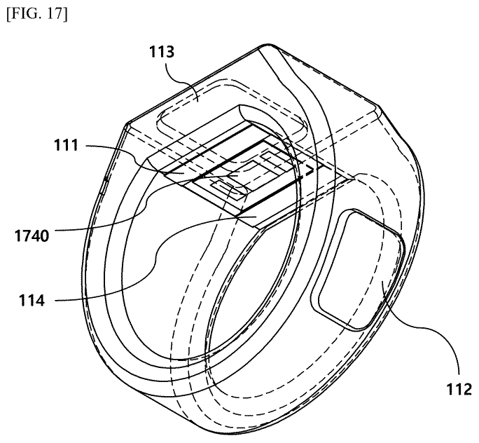

For user convenience and accuracy of ECG measurement, the locations of the electrocardiogram electrodes are important. A plurality of examples of implementing the electrocardiogram measurement apparatus according to the present invention on a watch will be described with reference to FIG. 16.

In the first example, three ECG electrodes may be installed on both sides of a watch band. In FIG. 16, one ECG electrode 111 is installed on the inner surface of the band, i.e., the surface of the band contacting the wrist, and the two electrodes 112 and 113 are installed on the outer surface of the band, i.e., the surface of the band that does not contact the wrist. In this example, when the user wears the watch on the left wrist, the electrode 111 contacts the left wrist. In this case, the user brings the electrode 112 into contact with the left lower abdomen or chest and the right hand finger into contact with the electrode 113 to perform ECG measurement.

In the second example, one ECG electrode 1610 may be installed on the bottom surface of the watch. In this case, the electrode 1610 is always in contact with the wrist wearing the watch. When the user is to measure the ECG, the electrode 112 is brought into contact with the left lower abdomen or chest, and the electrode 113 is brought into contact with one finger of the hand without the watch.

In the third example, another part of the watch body, for example 1640, may be used instead of the electrode 113 of FIG. 16.

In all the above cases where electrodes are installed on a watch or watch band for user convenience and accuracy of electrocardiogram measurement, it should be noted that one electrode 112 is installed on the outer surface, that is, the surface of the band that does not contact the wrist, of a portion of the band located on the inside of the wrist (the palm side, not the back side of the hand). This is intended to make the electrode 112 comfortably contact the user's left lower abdomen or chest portion. In addition, in all the above cases where electrodes are installed on a watch or watch band, the PPG 1630 installed on the bottom surface of the watch may analyze the PPG signal and generate an alarm to the user.

The electrocardiogram measurement apparatus according to the present invention may be implemented in a ring shape. In this case, the ring is worn on the thumb or little finger to facilitate electrocardiogram measurement. FIG. 17 shows an example in which the electrocardiogram measurement apparatus according to the present invention is implemented in a ring shape. In FIG. 17, one electrode 111 among the three electrodes contacts a finger wearing the ring. The electrode 112 and electrode 113 are not in contact with the finger. That is, the electrode 112 and the electrode 113 are located on the outer portion of the thumb or little finger, and are arranged spaced apart from each other. When the ring is worn on the thumb of the left hand, the electrode 111 may be brought into contact with the thumb of the left hand, the electrode 112 may be brought into contact with the lower left abdomen, and the electrode 113 may be brought into contact with the second finger of the right hand. PPG 1730 installed on the surface of the ring that touches the skin may analyze the PPG signal and generate an alarm to the user.

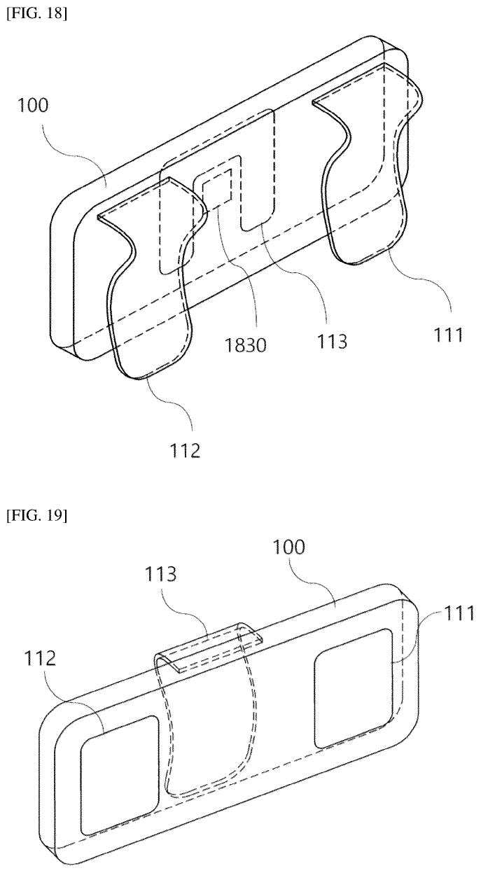

The electrocardiogram measurement apparatus according to the present invention may be implemented in a form that is easy to be coupled to other objects to keep the apparatus worn on a body. FIGS. 18 and 19 show examples of the electrocardiogram measurement apparatus according to the present invention that can be coupled to pants and measure an electrocardiogram immediately when the electrocardiogram is be measured. In FIG. 18, two clips 111 and 112 serving as two electrodes are used to attach the electrocardiogram measurement apparatus 100 according to the present invention to the inside of the pants, that is, between the pants and the user's body. When used, the electrocardiogram measurement apparatus 100 is attached to the pants at the position of the lower left abdomen using the clips 111 and 112. Then, the electrode 113 and the PPG 1830 automatically contact the lower left abdomen of the user. When the PPG 1830 sends an alarm or ECG measurement is needed, the user brings a left hand finger into contact with the clip 111 and brings a right hand finger into contact with the clip 112.

The electrocardiogram measurement apparatus 100 according to the present invention shown in FIG. 19 is attached to the outside of the pants. The electrocardiogram measurement apparatus 100 and the clip 113 inside the pants press the pants, and thus the electrocardiogram measurement apparatus 100 is fixed to the pants. When the electrocardiogram is measured, the clip 113 automatically contacts the user's lower left abdomen, and the user brings a left hand finger into contact with the electrode 111 and brings a right hand finger into contact with the electrode 112.

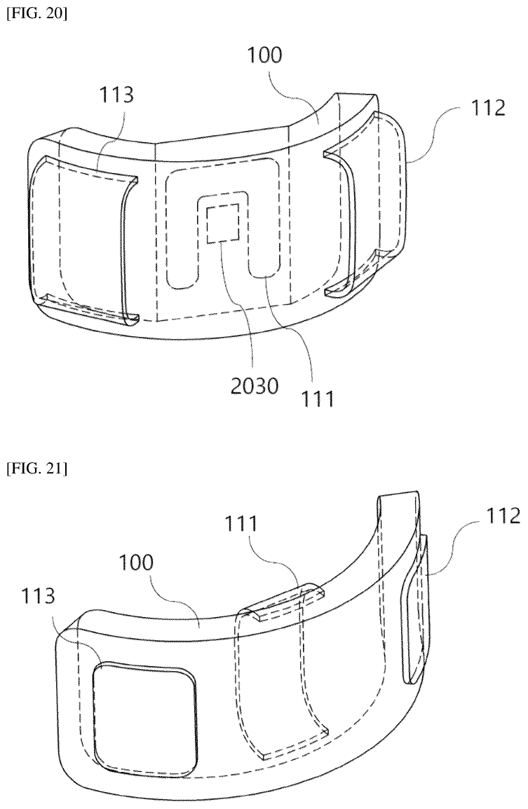

FIGS. 20 and 21 show embodiments of the electrocardiogram measurement apparatus that can be coupled to the band of a watch using two or one slide guide that serves as an electrode according to the present invention. In FIG. 20, when a watch band is inserted between the electrocardiogram measurement apparatus 100 according to the present invention and the slide guides 112 and 113 serving as electrodes, the electrocardiogram measurement apparatus 100 is fixed to the watch band. When the watch is worn on the left hand, the electrode 111 and the PPG 2030 automatically contact the left wrist. When the PPG 2030 sends an alarm or ECG measurement is needed, the user brings the lower left abdomen into contact with the slide guide 113 and brings a right hand finger into contact with the slide guide 112.

In FIG. 21, when the band of the watch is inserted between the electrocardiogram measurement apparatus 100 according to the present invention and the slide guide 111, the electrocardiogram measurement apparatus 100 is fixed to the band of the watch. When the watch is worn on the left hand, the electrode 111 automatically contacts the left wrist. In order to measure the ECG, the user brings the lower left abdomen into contact with the electrode 113 and brings a right hand finger into contact with the electrode 112.

As described above, the electrocardiogram measurement apparatus according to the present invention to which the PPGs 1830 and 2030 of FIGS. 18 and 20 are added is capable of constantly monitoring a user's heart rate. Although a separate drawing is not added for simplicity, it is apparent that the electrocardiogram measurement apparatus according to the present invention can be coupled to the band of a watch using the clips shown in FIG. 18 or 19 instead of the slide guides shown in FIGS. 20 and 21.

In the embodiment of the electrocardiogram measurement apparatus according to the present invention, the electrocardiogram measurement apparatus 100 is described as including three electrodes. However, in another embodiment according to the present invention, the electrocardiogram measurement apparatus may include four electrodes. The operation principle of an electrocardiogram measurement apparatus including the four electrodes according to the present invention is the same as that of the previous case of including three electrodes. The important point is that the electrocardiogram measurement apparatus including four electrodes according to the present invention includes three amplifiers configured to receive an ECG signal from three electrodes, the three amplifiers each amplify one ECG signal, and accordingly the apparatus actually measures three ECG signals simultaneously.

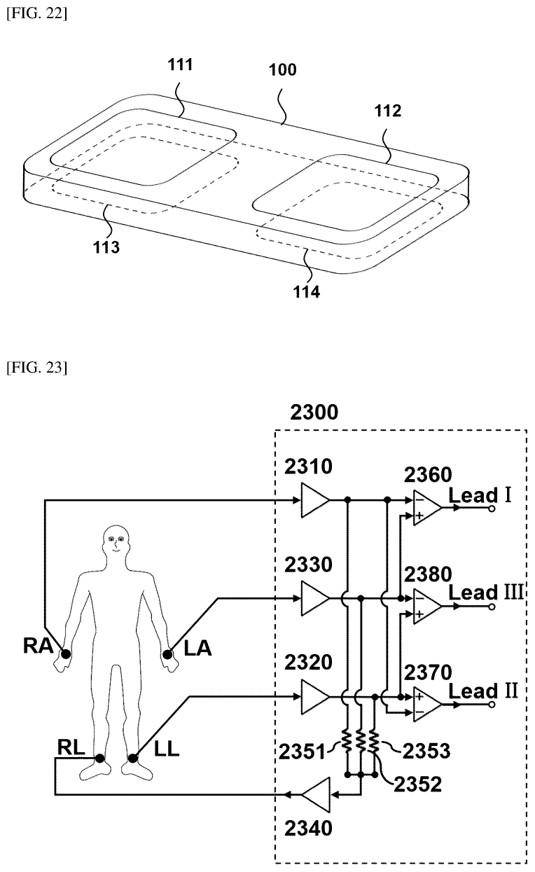

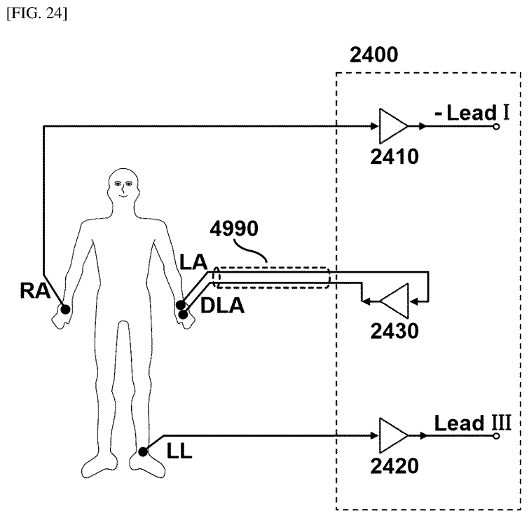

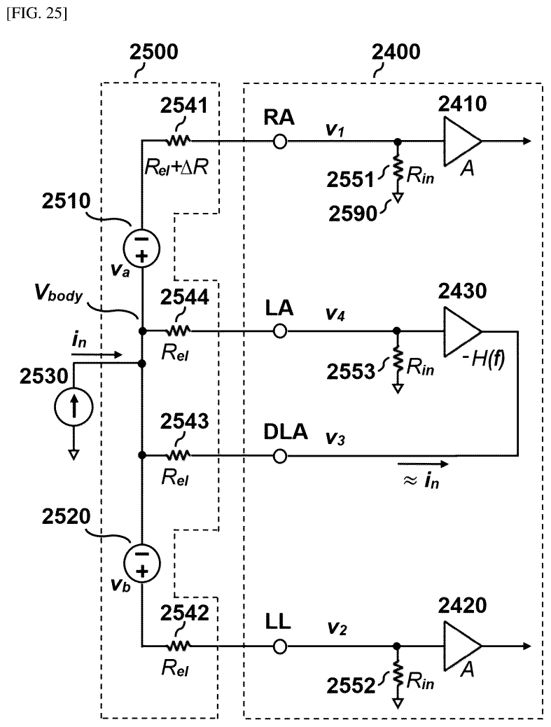

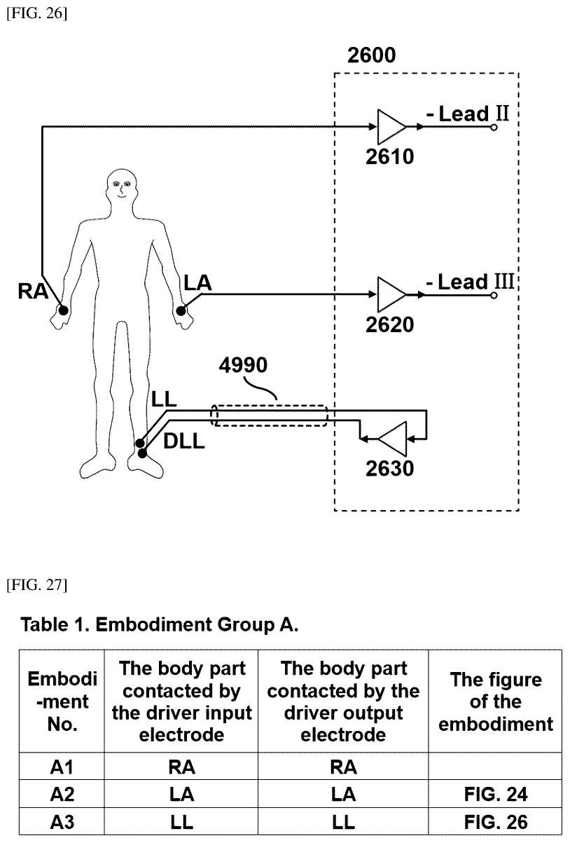

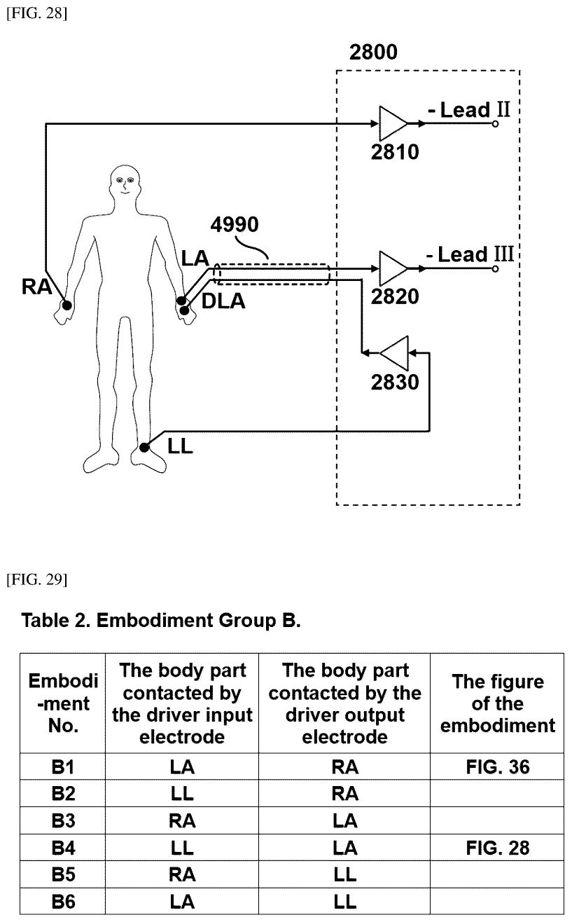

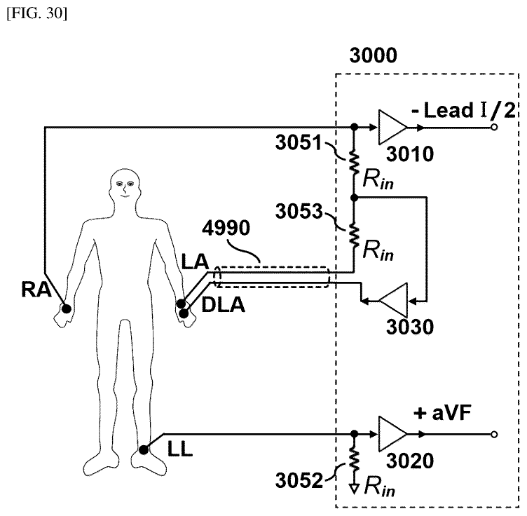

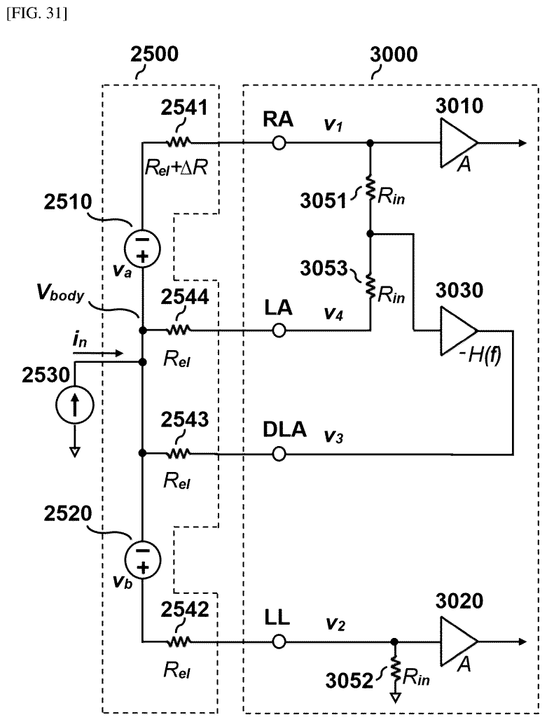

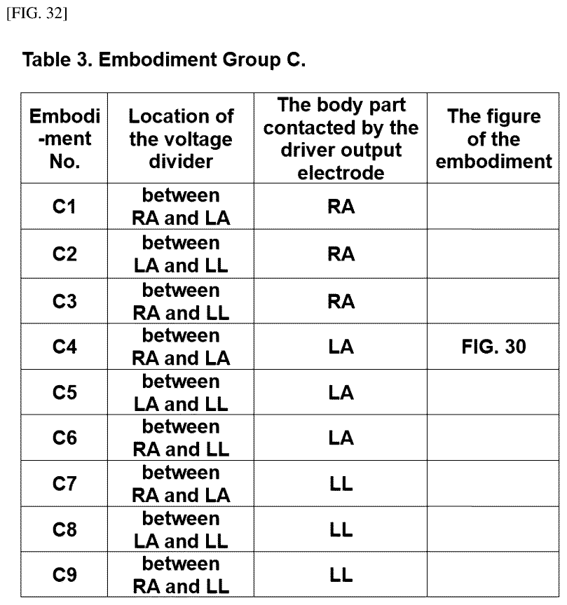

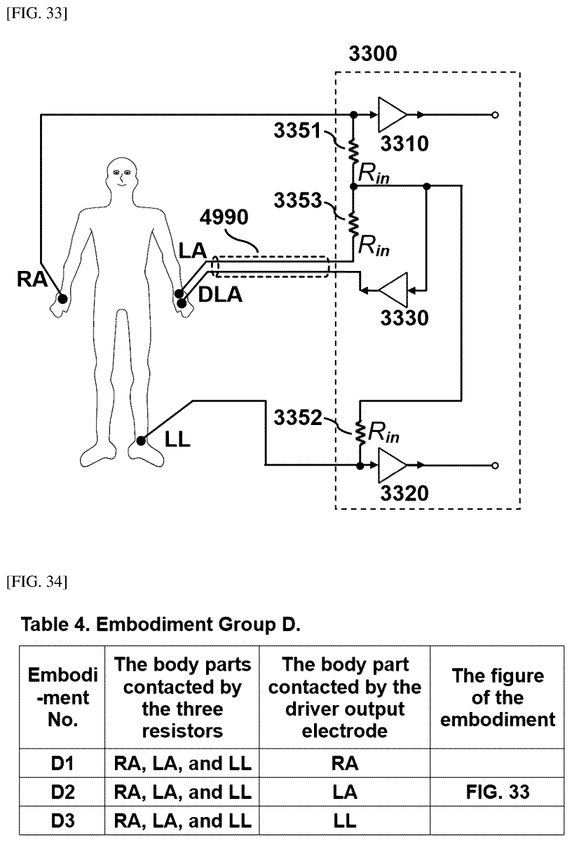

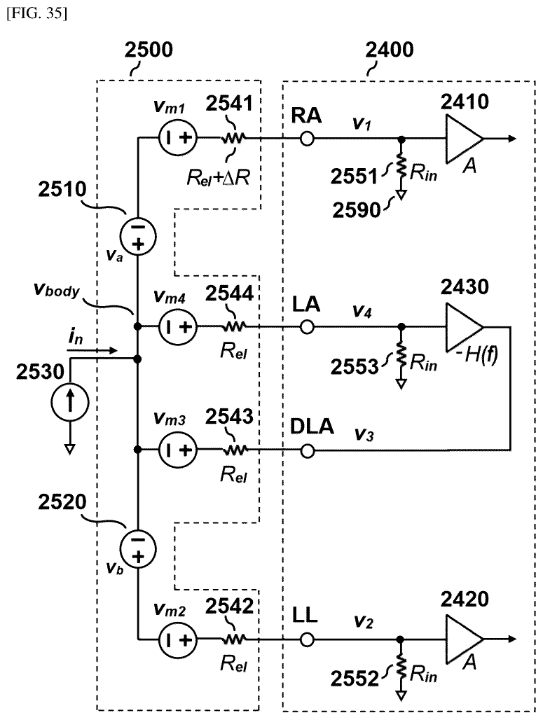

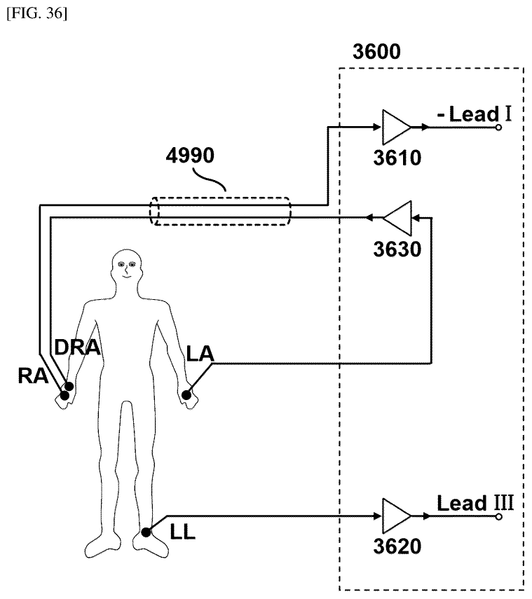

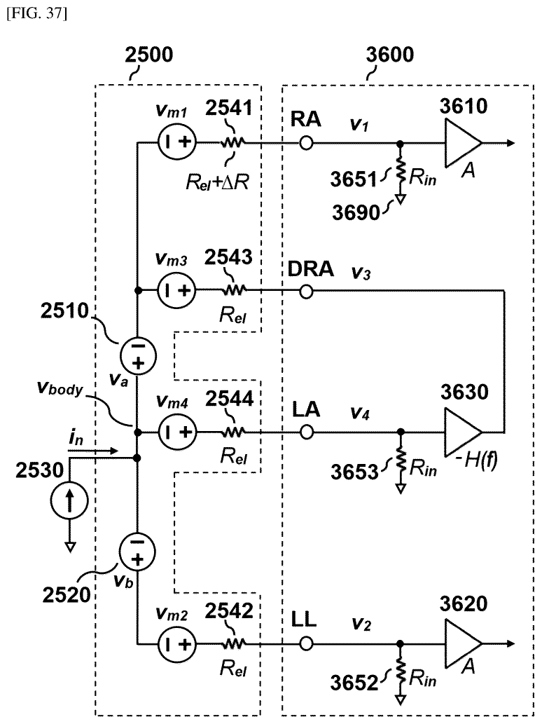

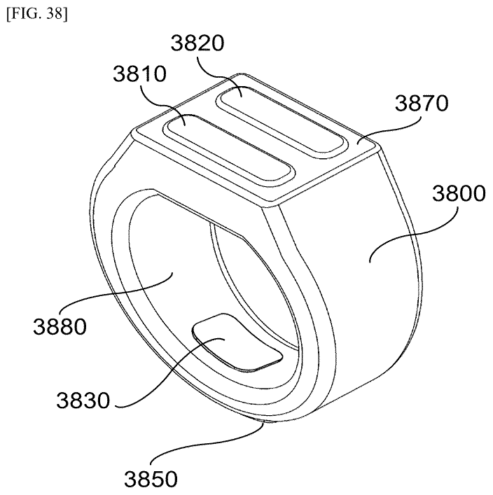

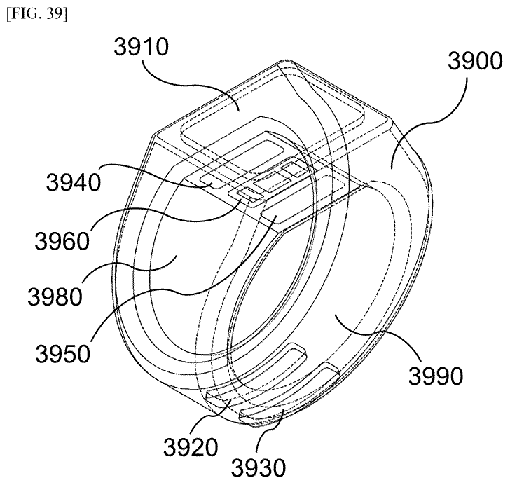

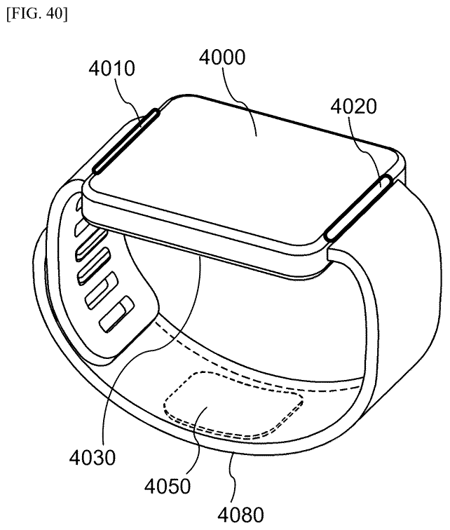

The electrocardiogram measurement apparatus including the four electrodes may be easily implemented by the foregoing description. The method of using the electrocardiogram measurement apparatus including the four electrodes according to the present invention is almost the same as the method of using the electrocardiogram measurement apparatus 100 including the three electrodes according to the present invention. The three ECG signals measured by the electrocardiogram measurement apparatus including four electrodes according to the present invention include, for example, two limb leads and one MCL. Alternatively, the three ECG signals may be one limb lead and two MCLs. An embodiment of the electrocardiogram measurement apparatus including the four electrodes according to the present invention is illustrated in FIG. 22. In FIG. 22, the four electrodes 111, 112, 113, and 114 are provided on two plate-shaped wide surfaces, two on each wide surface.

The electrocardiogram measurement apparatus according to the present invention has been described in detail, but the present invention is not limited thereto. The present invention may be changed in various forms according to the intention of the present invention.

The embodiments described from now on are based on HWANG (Application No.: KR10-2023-0003099, Filing Date: Jan. 9, 2023) filed with the Korean Intellectual Property Office and HWANG (Provisional Application No: 63/444,228, Filing Date: Feb. 9, 2023) filed with the United States Patent and Trademark Office. Also, some embodiments described hereafter are based on designs already registered in the European Patent Office and the US Patent and Trademark Office. Prior arts related to the embodiments described hereafter are as follows.

Chan et al. (U.S. Pat. No. 7,894,888 B2, Date of Patent: Feb. 22, 2011, H. Chan, et al.) used three differential amplifiers for electrocardiographs implemented in a watch measuring Lead I, Lead II, and Lead III. Chan et al. used three electrodes for this.

For a handheld device using four electrodes, Amitai et al. (U.S. Pat. No. 10,092,202 B2, Date of Patent: Oct. 9, 2018, D. Amitai, et al.) measured three differential voltages. That is, Amitai uses three differential amplifiers. Also, Amitai uses the Wilson Central Terminal.

The configuration for generating the voltage of Wilson Central Terminal is shown in FIG. 3B of VAJDIC et al. (US Pub. No.: US 2022/0125363 A1, Pub. Data: Apr. 28, 2022, B. VAJDIC, et al.). To generate the voltage of the Wilson Central Terminal, three buffer amplifiers, and three matched resistors are used to equally receive the three ECG voltages induced from the three electrodes contacting the right hand, left hand, and left leg.

A summary of the prior art described above is shown in FIG. 23. FIG. 23 shows a conventional technique for a measuring device for obtaining six electrocardiogram lead signals. The Wilson Central Terminal is implemented using three matched resistors connected to the outputs of the three buffer amplifiers. The voltage of the Wilson central terminal is applied to the input of one electrode driver 2340, and the output of the electrode driver 2340 is applied to the right leg electrode (RL: Right Leg) contacting the right leg. Lead I, Lead II, and Lead III are obtained by amplifying three differential voltages between right arm (RA), left arm (LA), and left leg (LL) or left lower abdomen using three differential amplifiers, respectively. For convenience, the left lower abdomen will be omitted from the description. From now on, left leg means left leg or left lower abdomen.

FIG. 23 shows a representative prior art. However, there are also conventional arts using two differential amplifiers. Charkravarthi et al. (US Pub. No.: US 2021/0113108 A1, Pub. Date: Apr. 22, 2021, V. S. Charkravarthi, et al.) discloses a device using a small number of probes, that is, electrodes. Charkravarthi et al. used Wilson Central Terminal and two differential amplifiers. However, Charkravarthi et al applied the signal of the Wilson Central Terminal to the right leg (RL).

R. F. A. Santala et al. (U.S. Pat. No. 10,405,765 B2, Date of Patent: Sep. 10, 2019, R. F. A. Santala, et al.) disclosed two differential amplifiers connected to three electrodes contacting the right hand, left hand, and left leg and a right leg driver (RLD) which applies its output to the electrode contacting the right leg. Thus, Santala et al. used four electrodes, two differential amplifiers, and one electrode driver. Differential voltages were measured by connecting a right arm (RA) electrode to a negative input terminal of each differential amplifier. On the other hand, Santala et al. did not use the Wilson Central Terminal. Santala et al. received an input of a right leg driver (RLD) from a right hand (RA) electrode and applied the output of the right leg driver to a right leg (RL) electrode.

The embodiments described hereafter are based on the prior invention of the present inventor (WO2019/108044A1, International Publication Date: Jun. 6, 2019, International Application No.: PCT/KR2018/015193, International Application Date: Dec. 3, 2018) and may be viewed as further embodiments using the further concept.

In the present invention from now on, the prior invention described above will be simply referred to as three electrode embodiments for convenience, if necessary. In the present invention, from now on, the differences from the above prior invention will be mainly described. Therefore, matters not described in the present invention described hereafter can be applied to the contents described in the three electrode embodiments (i.e., the contents described in the previous part of the present application). Thus, the present invention described hereafter includes the contents described above.

The appearance, operation principle, configuration, and use method of the electrocardiogram device according to the present invention for the above problems to be solved are as follows. The present invention solves the above problems through systematic and analytical circuit design and software production.

In accordance with one aspect of the present invention, provided is an electrocardiogram measuring device comprising:
a first electrode and a second electrode configured to receive a first and a second electrocardiogram voltages of a first and a second body part in contact therewith, respectively;
two amplifiers configured to receive the first and the second electrocardiogram voltages from the first and the second electrodes;
a third electrode configured to contact a third body part and transfer a third electrocardiogram voltage of the third body part;
an electrode driver configured to receive the third electrocardiogram voltage and output a driving voltage;
a fourth electrode placed adjacent to one of the three electrodes and configured to receive and transmit the output of the electrode driver to one of the three body parts in contact therewith;
an AD converter connected to an output terminal of each of the two amplifiers to convert output signals of the two amplifiers into two digital signals;
a microcontroller configured to receive the two digital signals of the AD converter; and
a communication means configured to transmit the two digital signals;
wherein:
the microcontroller is supplied with power from a battery;
the microcontroller controls the AD converter and the communication means; and
the two amplifiers each receive and amplify one electrocardiogram voltage simultaneously.

In accordance with one aspect of the present invention, provided is an electrocardiogram measuring device comprising:
a first electrode, a second electrode, and a third electrode configured to receive a first, a second, and a third electrocardiogram voltages of a first, a second, and a third body part in contact therewith, respectively;
three amplifiers configured to receive the first, the second, and the third electrocardiogram voltages from the first, the second, and the third electrodes;
a fourth electrode configured to contact a fourth body part and transfer a fourth electrocardiogram voltage of the fourth body part;
an electrode driver configured to receive the fourth electrocardiogram voltage and output a driving voltage;
a fifth electrode placed adjacent to one of the four electrodes and configured to receive and transmit the output of the electrode driver to one of the four body parts in contact therewith;
an AD converter connected to an output terminal of each of the three amplifiers to convert output signals of the three amplifiers into three digital signals;
a microcontroller configured to receive the three digital signals of the AD converter; and
a communication means configured to transmit the three digital signals;
wherein:
the microcontroller is supplied with power from a battery;
the microcontroller controls the AD converter and the communication means; and
the three amplifiers each receive and amplify one electrocardiogram voltage simultaneously.

According to the present invention, two electrocardiogram voltages are measured using two amplifiers having high input impedance that receive the two electrocardiogram voltages from two electrodes while removing power line interference by applying an output of one electrode driver to one electrode. An embodiment of simultaneously measuring two electrocardiogram voltages is described.

The most significant feature of the three electrode embodiments is that two amplifiers including at least one single-ended input amplifier are used to sense two ECG lead signals induced to the first and second electrodes among the three electrodes. Also, the output of one electrode driver is applied to the third electrode among the three electrodes. This is to obtain an effect of reducing power line interference by allowing substantially all current of the power line interference current source to flow through the third electrode by connecting an electrode driver output terminal having a low output impedance to the third electrode.

After simultaneously measuring two ECG lead signals, four ECG lead signals are additionally calculated and obtained. The additionally obtained four ECG lead signals can be displayed on a smartphone. Embodiments to be described from now on will focus on simultaneously measuring two ECG lead signals.

The above characteristics of the three electrode embodiments are applied as they are to the electrocardiogram measuring device according to the present embodiments using four electrodes. The device according to the following embodiments is an embodiment in which one electrode is added to the device according to the three electrode embodiment. All other required components and usage are the same. The device according to this embodiment is suitable for implementation as a wearable device such as a watch, a ring, a patch shape, or a chest strap.

In order to reduce power line interference in the three electrode embodiment, since the input impedance that the power line interference current source looking into the electrocardiograph through the human body must be reduced, an electrode driver output terminal having a low output impedance is connected to one electrode. A four electrode embodiment to be described also has the same common feature.

In the present invention, the goal is to achieve compliance with the standard of [ANSI/AAMI/IEC 60601-2-25:2011, Medical electrical equipment-part 2-25: Particular requirements for the basic safety and essential performance of electrocardiographs], which is an international standard for electrocardiographs. The goal is obtaining the electrocardiogram voltages defined by the standard, but implementing a simple and convenient device. An object of the present invention is to implement a wearable device that is small, lightweight and consumes little power.

The embodiments described hereafter use four electrodes but contact three places on the human body. When a device has four electrodes, it can contact four places on the human body, but it is convenient to contact three places: right hand, left hand, and left leg with the wearable device. It is very inconvenient to touch four parts of the human body with one wearable device.

In addition, in all embodiments to be described from now on, negative feedback is used using one electrode driver. The frequency band of the electrode driver used in all embodiments includes the power line interference frequency and the frequency band of the electrocardiogram signal according to ANSI/AAMI/IEC 60601-2-47:2012, that is, at least from 0.67 Hz to 40 Hz. ST segment measurement or recording ECGs from infants can be performed if the frequency band of the electrode driver includes 0.67 Hz or lower. In addition, the additional effect of reducing baseline wandering can be obtained.

Briefly describing the present invention: A wearable electrocardiograph using four electrocardiogram electrodes, each of which contacts one of the three body parts. The wearable electrocardiograph comprises two electrodes connected to two amplifiers, one electrode connected to an input terminal of one electrode driver, and one electrode connected to an output terminal of the electrode driver.

Compared to the embodiment using the three electrodes described above, the characteristics of the embodiment using the four electrodes described below are briefly summarized as follows.

Measures two ECG lead signals induced at two electrodes.

The output of one electrode driver is applied to the third electrode.

Obtains six electrocardiogram lead signals.

A difference from the three electrode electrocardiograph is that one electrode is added.

Connects the added electrode to the input terminal of the electrode driver for a better power line reduction.

Compared to the prior art, the superiority and differences of the embodiments described hereafter are described as follows.

The present invention includes an embodiment in which two single-ended input amplifiers are used instead of two differential amplifiers. The differential amplifiers shown in FIG. 23, used for electrocardiogram measurement in the prior art are instrumentation amplifiers. An instrumentation amplifier is a differential amplifier with a high input impedance and a low output impedance. An instrumentation amplifier is typically implemented using three operational amplifiers and four matched resistors. In contrast, a single-ended input amplifier used in the present invention is implemented with one operational amplifier. Therefore, the differential amplifier shown in FIG. 23 is significantly more complicated, larger in size, more expensive, and consumes more power than the single-ended input amplifier used in the present invention.

It is very well known that one single-ended input amplifier used in the present invention is implemented with one operational amplifier. Meanwhile, a differential amplifier in which one input terminal among two input terminals of a differential amplifier is connected to a circuit common, that is, signal ground, operates as a single-ended input amplifier. Therefore, one single-ended input amplifier in the present invention includes one differential amplifier or one instrumentation amplifier with one input terminal connected to the circuit common. Connecting one input terminal of the differential amplifier to the circuit common includes connecting the input terminal of the differential amplifier to the circuit common through an element having one high impedance.

In addition, it is described later that the voltage of the electrode connected to the input of the electrode driver becomes nearly zero, that is, it becomes a virtual ground. Therefore, a differential amplifier with one input terminal connected to the input of an electrode driver can be regarded as a single-ended input amplifier. Therefore, in the present invention, one single-ended input amplifier includes one differential amplifier having one input terminal connected to the input of the electrode driver. Here, one differential amplifier includes one instrumentation amplifier.

In the prior art, using three differential amplifiers to measure the three ECG lead signals of lead I, lead II, and lead III is a redundant implementation. This is because if two of the three ECG lead signals are known, the other one can be calculated. Therefore, using three differential amplifiers is less efficient than using two differential amplifiers. Therefore, using two single-ended input amplifiers in the present invention is more efficient than using three differential amplifiers.

As described later, even when a differential amplifier is used, induced motion artifacts are the same as when a single-ended input amplifier is used. This is another reason to use a single-ended input amplifier in the present invention.

ii) The traditional Wilson Central Terminal is not used in the present invention. In the embodiments of the present invention, the input signal to the electrode driver is received from one electrode. Or, in other embodiments of the present invention, in order to simplify the traditional Wilson Central Terminal, a buffer amplifier is not used and implemented with only two or three resistors.

In the prior art, to use Wilson Central Terminal, the output of the electrode driver was applied to the right leg (RL) in order to measure chest leads as well as limb leads in an environment where power line interference is very strong, such as a hospital operation room. The prior art is very inefficient from the viewpoint of the present embodiments because the present embodiments locate the RL electrodes at a different position suitable for a wearable electrocardiogram device. Therefore, the prior art of using the Wilson central terminal and applying the output of the electrode driver to RL is merely adding elements that are not practically necessary, although it could have had some benefits in the prior art. Therefore, the prior art is less efficient than the present invention when implementing a wearable electrocardiograph.

iii) In the present invention, the output of the electrode driver is not applied to the right leg. In the present invention, the output of the electrode driver is applied to one body part among the right hand, left hand, and left leg as required. Not applying the output of the electrode driver to the right leg is an absolutely essential condition for the miniaturization of wearable devices. If an electrode of a wearable device is to be in contact with the right leg, one cable and one electrode connected to one end of the cable must be used. This is very inconvenient to use and increases the size of the wearable device unnecessarily. In the present embodiments, the output of the electrode drive can be applied to one of RA, LA, and LL depending on an embodiment. The present embodiments disclose all possible embodiments for obtaining six ECG leads using four electrodes.

iv) Santala et al. receive the input of the electrode driver from RA. In the present embodiments, the input of the electrode drive can be received from LA or LL instead of RA. In the present embodiments, the input of the electrode drive can be received from one of RA, LA, and LL depending on an embodiment, and all possible embodiments for obtaining six ECG leads using four electrodes are disclosed.

v) Santala et al. connected the inputs of two differential amplifiers to RA while receiving the input of an electrode driver from RA. That is, the input of the electrode driver was connected to the two inputs of the two differential amplifiers. That is, the input of the electrode driver was connected to two amplifiers. However, in the present embodiments, the input and output of one electrode driver are not used to measure the ECG lead signals. That is, the two electrodes connected to the input and output of the electrode driver are not connected to the input of the amplifier used for measurement. Among the four electrodes, only two electrodes that are not connected to the input or output of the electrode driver are connected to two amplifiers to measure two ECG lead signals.

vi) The present invention targets various types of wearable devices, such as a plate shape, a watch, a ring, a patch shape, and the like. Therefore, in the present invention, the four electrodes are disposed to suit the structure, manufacturing method, and usage method of each wearable device. To this end, electrode arrangement rules according to the present invention are provided. This is described in more detail below.

vii) Among the matters to be further considered in arranging ECG electrodes, motion artifacts should be considered. Motion artifacts are noise generated when the relative position and pressure of one electrode and the skin in contact with the electrode change. Since the motion artifacts are difficult to remove once generated, it is a particularly troublesome problem in wearable devices. Compared to electrocardiographs for hospitals that measure with wet electrodes attached to the skin in a comfortable lying and resting state, motion artifacts are generated easily in wearable devices in which dry electrodes must contact preset body parts according to the user's will in an unfixed position. However, it is very important to reduce motion artifacts for accurate and comfortable ECG readings.

The four electrodes installed on the wearable device are inevitably affected by motion artifacts. Motion artifact is a major cause of baseline wandering. A human body motion may result in the baseline wandering in the electrocardiogram. Baseline wandering noise is just as prone to wearable devices as motion artifacts. Though it is also difficult to remove baseline wandering noise in wearable devices, reducing baseline wandering noise is important. In the present invention, a rule for reducing motion artifacts is prepared and embodiments according to the rule are disclosed.

So far, it has been described that a single-ended input amplifier is used to implement the present invention. In the present invention, using a single-ended input amplifier is more advantageous than using a differential amplifier in terms of size, price, and power consumption. However, it is possible to use a differential amplifier instead of a single-ended input amplifier to implement the present invention. In the present invention, all embodiments using single-ended input amplifiers can be replaced with embodiments using differential amplifiers without additional requirements. Therefore, even if a differential amplifier is not specifically mentioned or illustrated, the present invention includes an embodiment using a differential amplifier.

Further consideration is given below to the difference between the use of a differential amplifier and the use of a single-ended input amplifier in the present invention.

To sense one differential voltage, one differential amplifier must be used. In one differential amplifier, two signals are input to the two input terminals of + input terminal and − input terminal respectively. That is, two ECG electrodes must be connected to one differential amplifier. Therefore, at least three ECG electrodes must be connected to the two differential amplifiers. Thus, one of the three ECG electrodes is connected to the two input terminals of the two differential amplifiers. Now, the input and output of the electrode driver must be connected to two electrodes.

Taking Santala et al. as an example, the inputs of the two differential amplifiers are commonly connected to the RA electrode. When the electrode driver operates, the input of the electrode driver becomes a virtual ground. This will be described later in the embodiments of the present invention. That is, when the input of the electrode driver is connected to the RA electrode, the voltage of the RA electrode becomes substantially zero Volt. Therefore, the two inputs of the two differential amplifiers commonly connected to the RA electrode are effectively zero Volt. Therefore, using a differential amplifier is not much different than using a single-ended input amplifier.

On the other hand, in the above embodiments, consider the case of changing the input and output of the electrode driver. That is, consider the case where the output of the electrode driver is applied to the right-hand electrode (to distinguish from RA, it will be called DRA for convenience) and the electrode driver's input is received from another right-hand electrode (to be called RA). However, in this case, since the power line interference voltage is very high at the output of the electrode driver, a strong power line interference is applied to the DRA electrode connected to the electrode driver output and the input terminals of the differential amplifiers. That is, the signal-to-noise ratio of the ECG signal output from the differential amplifiers is considerably lowered. Therefore, in this case, if two differential amplifiers are used, rather than obtaining a good electrocardiogram signal from which power line interference is removed, a considerably bad result is inevitable. Therefore, the input of the differential amplifier must be connected to the input of the electrode driver and not to the output of the electrode driver.

The purpose of the embodiments described hereafter is to simplify and optimize the prior art shown in FIG. 23 to be suitable for wearable devices. Embodiments according to the present invention described from now on will be described by dividing them into several groups. One group has commonalities and there are differences between different groups.

Embodiments from now on will be referred to as Embodiment Group A. In Embodiment Group A, the input and output electrodes of the electrode driver are in contact with the same body part.

In the electrocardiogram measuring device including four electrocardiogram electrodes described below and the electrocardiogram measuring device including three electrocardiogram electrodes described above, the three electrodes commonly contact the right hand, left hand, and left leg. An electrocardiogram measuring device including four electrocardiogram electrodes includes one additional electrode compared to the electrocardiogram measuring device including three electrocardiogram electrodes described above.

One electrode to be added is connected to the input of one electrode driver. Since what is pursued in the present invention is a wearable device, the additional electrode should also contact one of the right hand, left hand, and left leg. Since four ECG electrodes are in contact with three body parts, two electrodes are in contact with one of the right hand, left hand, and left leg. The contact position of the two electrodes is selected according to the application, that is, according to the device. That is, the position of the electrode connected to the input of the electrode driver varies depending on the embodiment. The position of the electrode connected to the input of the electrode driver should be selected as a position where less noise is generated in the ECG signal.

Assume a situation where three electrodes are installed before adding one additional electrode. Two amplifier inputs and an electrode driver output are connected to three places on the human body. That is, one device is already connected to each of the three body parts. Now one additional electrode must be selected as the electrode driver input. Therefore, it is conceivable that one additional electrode may be installed at any of the three body parts and the electrode driver input is connected to the additional electrode. But it is not. The reason for this is as follows.

Electrode placement rules start with understanding the following. In the present invention using four electrodes, one electrode driver is an amplifier having a considerably high input impedance. As described further later, the input of the electrode driver becomes electrically a virtual ground. That is, the voltage of the input terminal of the electrode driver becomes near zero.

Since the electrode driver input has a high impedance, the voltage of the electrode driver input is the same as the voltage of the body part contacting with the electrode connected to the input of the electrode driver. That is, the electrocardiogram voltage of the body part contacting the electrode connected to the input of the electrode driver becomes zero. Therefore, if another electrode contacts the body part contacting the electrode driver input electrode and an amplifier input is connected to the electrode contacting the body part, the electrocardiogram voltage input to the amplifier becomes zero. Therefore, important conclusions are drawn.

Electrode Placement Rule 1: An electrode connected to the input of an ECG measurement amplifier cannot be installed or placed at the same location as an electrode driver input electrode is installed or placed.

Electrode Placement Rule 2: Considering the above Electrode Placement Rule 1, the electrode connected to an electrode driver input must be placed on the same body part as the electrode to which the electrode driver output is connected.

Accordingly, the input electrode and the output electrode of the electrode driver are installed on the same body part. However, there is no limitation on the position where the electrode driver input and output are installed. Therefore, two electrodes connected to the input and output of the electrode driver can be installed anywhere on the right hand, left hand, or left leg. That is, there are three methods of installing two electrodes connected to the input and output of the electrode driver. Accordingly, the position of the electrode for sensing the two ECG signals must also be changed. This is a new concept that cannot be inferred from the method of Santala et al.

An embodiment according to the present invention will now be described with reference to the figures. In this embodiment, the electrocardiogram measuring device is described as including four electrodes but is not limited thereto, and the electrocardiogram measuring device may include five or more electrodes.

All figures used in the embodiments of the present invention may show measurement circuits of the electrocardiogram measuring device according to the present invention implemented as a wearable. The importance of electrical equivalent circuit modeling cannot be overemphasized. The development of the electrocardiogram measuring device starts from electrical equivalent circuit modeling, and the content to be improved must be based on this.

FIG. 24 shows one embodiment of the present invention. In the embodiment of FIG. 24, two ECG lead signals, Lead I, and Lead III, are each sensed by a single-ended input amplifier. The output of electrode driver 2430 is applied to electrode DLA. The difference between the present invention and the three electrode invention is that one electrode is added and the added electrode is connected to the input of the electrode driver. In the embodiment of FIG. 24, a LA electrode is added, and the LA electrode is connected to the input of the electrode driver 2430.

It can be seen that the embodiment of FIG. 24 is very simple compared to the prior art of FIG. 23. In FIG. 24 and the following figures, 4990 indicates that a single cable can be bundled when two connection lines are used to bring two electrodes into contact with the human body in order to implement the present invention as a Holter-type electrocardiograph or an electrocardiograph sing cables. This provides convenience and avoids confusion about cables. Meanwhile, when the device according to the present invention is implemented as a wearable device, it indicates that it can be placed on one body part. For example, when the device of FIG. 24 is implemented as a device included in a watch, it is possible to place two electrodes on the bottom of the watch in contact with the wrist wearing the watch. As another example, when the device of FIG. 24 is implemented as a device included in a ring, it is possible to place two electrodes on the inner surface of the ring that contacts the finger wearing the ring. The embodiment of FIG. 24 is suitable for implementation as a single watch or ring.

FIG. 25 shows an electrical equivalent circuit for analyzing the electrical operation of the embodiment of FIG. 24. In FIG. 25, for convenience, the RA electrode is shown at the top of the Figure, and the LL electrode is shown at the bottom of the Figure. Power line interference is caused by the power line interference current source 2530. The power line interference current source 2530 applies the power line interference current into the electrocardiogram measuring device 2400 according to the present invention through the body 2500 of a user. The device 2400 according to the present invention includes four electrodes RA, LA, DLA, and LL. The voltages of each electrode were expressed as v₁, v₄, v₃, and v₂. Two single-ended input amplifiers 2410 and 2420 are respectively connected to two ECG sensing electrodes, RA and LL. The input of one electrode driver 2430 is connected to electrode LA and the output is connected to electrode DLA. The transfer function of the electrode driver 2430 is represented by −H(f). The magnitude of the transfer function is considerably large in the power line interference frequency and the ECG lead signal frequency band.

The input impedance of the two amplifiers and one electrode driver is represented by Rᵢₙ. The four electrodes contact the human body 2500, and electrode impedance Rₑₗ exists between the skin and the electrodes. Usually, Rᵢₙ is very large. That is, Rᵢₙ>>Rₑₗ. Each electrode impedance value may be different. The difference between electrode impedance 2541 and electrode impedance 2542 was expressed as ΔR. In general, since the output impedance of the electrode driver is much smaller than the electrode impedance Rel, it is regarded as zero in many cases. The electrocardiogram voltage of electrode LA relative to electrode RA was expressed as vₐ. That is, vₐ is Lead I. The electrocardiogram voltage of electrode LL relative to electrode LA was denoted by v_{b}. That is, v_{b} is Lead III.

Analysis of the electrical equivalent circuit gives the following result. The voltage v_{body} induced to the human body by the power line interference current source 2530 becomes very small due to the influence of the electrode driver 2430. v_{body}=in*Rₑₗ/(H(f)+1) In the present invention, substantially all of the power line interference current flows through one electrode connected to the output of the electrode driver. This is the same as in the three electrode embodiments described above. In FIG. 25, substantially all of the power line interference current flows through one electrode DLA. Due to the influence of the electrode driver 2430, v₄ becomes very small. Effectively, v₄ is zero Volt. That is, v₄ is a virtual ground. It should be noted here that the virtual ground is for an AC signal, and v₄ may include a DC voltage component. A constant voltage source supplying a constant DC voltage is regarded as grounded in an alternating current circuit.

Consequently, v₁=−vₐ=−Lead I. Also, v₂=v_{b}=Lead III. That is, power line interference was removed, and two ECG lead signals, Lead I, and Lead III, were measured. In an embodiment of the present invention −Lead I is an inverted Lead I. In a wearable measurement device using one battery, analog signals are referenced to a common AC signal ground provided at an approximate middle value of the battery. A signal that is referenced to an AC signal ground is AD-converted to approximately the middle value of the AD-conversion range. The signal of a negative number only changes up and down with a reference at the middle of the AD conversion range. Therefore, negative values of the sensed or measured −Lead do not matter.

This is a major difference between an electrocardiogram measurement device that uses a common ground in an electronic circuit that uses a positive supply power and a negative supply power and AD converts based on a circuit common, that is zero DC voltage, and a wearable electrocardiogram measurement device that uses a single battery. This difference is notable. Due to this difference, various embodiments of the present invention are possible. In the present embodiments, it is possible to measure an inverted signal, for example, −Lead I rather than Lead I, so that various embodiments can be easily implemented.

FIG. 26 shows another embodiment of the present invention. The input and output of electrode driver 2630 are connected to the left leg. The signal sensed by the right hand electrode RA is −Lead II. The signal sensed by the left electrode LA is −Lead III.

The embodiment of FIG. 26 is advantageous for application when implementing an electrocardiograph attached to pants by the two clips shown in FIG. 18. When the electrocardiogram measuring device attached to the pants by the two clips described above is worn, the two electrodes, electrode LL and electrode DLL, maintain contact on the left lower abdomen even when an electrocardiogram is not measured. When measuring electrocardiograms, two hands are brought into contact with the two clips 111 and 112 of FIG. 18.

The embodiments including FIG. 24 and FIG. 26 described so far will be referred to as Embodiment Group A. There are three embodiments in Embodiment Group A, and they are summarized in Table 1 of FIG. 27. In Embodiment Group A, two electrocardiogram measuring electrodes are respectively disposed at two body parts where the input electrode and output electrode of the electrode driver do not contact.

Embodiments described from now on will be referred to as Embodiment Group B. In the embodiments of Embodiment Group B, the input electrode and the output electrode of the electrode driver are installed to contact different human body parts. The reason why Embodiment group B is possible is because of the following Electrode Placement rule 3.

Electrode Placement Rule 3: An electrode connected to the output terminal of the electrode driver can be placed anywhere on the three parts of the human body.

Therefore, the electrode driver output electrode can be placed at a body part contacted by another electrode connected to an electrocardiogram measurement amplifier. It is surprising that this is possible. One example of this is shown in FIG. 28. This is a new concept that is difficult to infer from the method of Santala et al. Before actually implementing Electrode Placement Rule 3, the following Electrode Placement Rule 4 may be used.

Electrode Placement Rule 4: Assign the electrode driver input electrode to the third part of the human body where the two electrocardiogram measuring electrodes are not installed. Now, the electrode driver output electrode may be freely assigned to a desired location among the three body parts.

The Electrode Placement Rules 3 and 4 may be expressed as follows. Dispose three electrodes on the right hand, left hand, and left leg, respectively, and connect the inputs of the three amplifiers to the three electrodes. Set one of the three amplifiers as an electrode driver. Connect the output of the electrode driver with a fourth electrode and assign the fourth electrode to the same human body part where one of the three electrodes is already disposed of.

Using the above Electrode Placement Rules, we get the following result. At the final stage of electrode placement, it is necessary to check the following Electrode Placement Rule 5.

Electrode Placement Rule 5: One more electrode is installed on a body part where one electrode driver output electrode is installed. That is, two electrodes are installed on the same body part. This can be expressed as when two electrodes are in contact with one body part, one of the two electrodes is necessarily the electrode driver output electrode.

FIG. 28 shows an embodiment of the present invention. The device in the embodiment of FIG. 28 senses ECG signals induced in the right and left hands. In the embodiment of FIG. 28, the measured ECG signals at the right hand and the left hand are the same as in the embodiment of FIG. 26. The electrode driver 2830 receives an input signal from the left leg and applies the output signal of the electrode driver 2830 to the left hand. Then, the ECG signal sensed from the right hand is −Lead II. The ECG signal sensed from the left hand is −Lead III. In the embodiment of the present invention, it can be seen from the embodiment of FIG. 28 that the input electrode and the output electrode of one electrode driver can be respectively connected to different parts of the human body. The embodiment of FIG. 28 has the feature that two electrodes are in contact with the left hand.

The embodiment of FIG. 28 is advantageous for fabrication over Embodiment Group C or Embodiment Group D using additional matched resistors. The electrical equivalent circuit model analysis is omitted.

So far, it has been found that contact positions of electrodes connected to the input and output of the electrode driver can be variously changed. As seen through the above Figures, the positions of the electrodes used in the present embodiments are limited to the right hand, left hand, left leg or left lower abdomen, or chest. Other than that, there is no limitation on the position of an electrode. Therefore, the number of embodiments is six in total, as specifically shown in Table 2 in FIG. 29. Although the six embodiments may be different in mechanical form, they all obtain two ECG lead signals in common. Depending on the embodiment in FIG. 29 and the position of the electrode, each electrode has a different impedance Rₑₗ and motion artifacts, which are further described later.

In order to simply call the electrodes connected to the input and output terminals of an electrode driver, respectively, they may be referred to as an electrode driver input electrode and an electrode driver output electrode.

A review of the embodiments of Embodiment Group B provides the following advantages.

It can be decided where to place the two electrodes on the body.

It can be decided which embodiment to choose to reduce motion artifacts as described later.

It is possible to review what possible embodiments exist.

In Embodiment Group B, one electrocardiogram measuring electrode is positioned at the same position as the electrode driver output electrode, and the other electrocardiogram measuring electrode is positioned at a position other than the electrode driver input and output electrodes.

So far, a single-ended input amplifier has been used to implement the present invention. However, as previously described, the present invention includes using a differential amplifier instead of a single-ended input amplifier. As described above, it is unnecessary to use a differential amplifier in the present invention. Using a single-ended input amplifier is more advantageous than using a differential amplifier in terms of size, cost, and power consumption.

However, it is possible to use a differential amplifier instead of a single-ended input amplifier to implement the present invention. In the present invention, all embodiments using single-ended input amplifiers can be replaced with embodiments using differential amplifiers as they are. Therefore, even if the differential amplifier is not specifically mentioned or illustrated, the present invention includes embodiments using a differential amplifier. For Embodiment Group B a differential amplifier may be used instead of a single-ended input amplifier. A method of using the differential amplifier in the present invention will be described later.

Embodiments from now on belong to Embodiment Group C. In Embodiment Group C, a voltage divider is constructed using two matched resistors, and an input signal of the electrode driver can be received from the intersection of the voltage divider.

FIG. 30 shows one embodiment belonging to Embodiment Group C of the present invention. In the embodiment of FIG. 30, two matched resistors are placed between the right hand electrode and the left hand electrode. The two resistors act as a voltage divider. The connection point of the two resistors is connected to the input terminal of the electrode driver. FIG. 30 is a case where the output of the electrode driver is applied to the left hand. In another embodiment, one voltage divider may be formed between two electrodes contacting different body parts.

FIG. 31 shows an electrical equivalent circuit model for analyzing the operation of the embodiment of FIG. 30. −Lead I/2 is sensed from the right-hand electrode. Lead I/2+Lead III=aVF is sensed from the left leg electrode. Lead I and Lead III are calculated from the two sensed lead signals. The calculation may be performed after AD conversion of the two sensed lead signals. Detailed descriptions are omitted.

The embodiment of FIG. 30 has the advantage of directly measuring aVF without calculation. In Embodiment Group C of a practical example, one differential amplifier can be used instead of one single-ended input amplifier as in the three electrode embodiment. For example, in the embodiment of FIG. 31, a single differential amplifier that receives inputs from RA and LA can be used instead of the single-ended input amplifier 3010. Embodiment Group C has nine embodiments, as shown in Table 3 in FIG. 32. Detailed descriptions are omitted.

Embodiments from now on will be referred to as Embodiment Group D. The resistors used in Embodiment Groups C and D have a high resistance of at least 10 MOhm or more. Compared to the past, it is easy to obtain commercially available small chip resistors or thin-film resistors of 10 MOhm or more these days. Matched resistors used in the prior art of FIG. 23 could typically use resistors on the order of 10 kOhm.

FIG. 33 shows another embodiment of the present invention that obtains the Wilson Central Terminal voltage using three matched resistors. The embodiment of FIG. 33 implements a Wilson Central Terminal simpler than the Wilson Central Terminal shown in the prior art of FIG. 23. In the embodiment of FIG. 33, the obtained Wilson Central Terminal voltage is applied to the LA electrode of the left hand. In this case, −2/3*Lead I−1/3*Lead III is sensed at the right hand amplifier 3310. At the left leg amplifier, 1/3*Lead I+2/3*Lead III is sensed. Lead I and Lead III are calculated from the two sensed values.

In FIG. 33, the output electrode of the electrode driver can be contacted anywhere on the right hand, left hand, or left leg. Thus, there are three embodiments of Embodiment Group D, as shown in Table 4 of FIG. 34.

In embodiments that implement the Wilson Central Terminal digitally, it is possible to avoid using three matched resistors. Instead of using three resistors, one additional ECG voltage can be obtained using the two ECG voltages measured at two electrodes, and then the Wilson central terminal voltage can be obtained by calculation.

Embodiments from now on will be referred to as Embodiment Group E. In Embodiment Group E, among the various embodiments described above, superior embodiments for reducing motion artifacts are described. As described above, for user convenience and accuracy of electrocardiogram measurements, the location and shape of the electrocardiogram electrodes installed in the electrocardiogram measuring device are important.

In the embodiments using four electrodes, one ECG electrode connected to the input of an electrode driver is added compared to the embodiments using three electrodes described above. From now on, among the various embodiments described above, the superior embodiments are selected to reduce motion artifacts. However, variations from the embodiments described below are possible depending on the purpose and method of use. Therefore, embodiments in which the Embodiments Groups A to D are changed to be similar to the embodiments described below are also included in the scope of the present invention.

Among the matters to be further considered in arranging ECG electrodes, motion artifacts may be considered. Motion artifacts are noise generated when the relative position and pressure of one electrode and the skin in contact with the electrode change. It is well known that motion artifacts are a particularly troublesome problem in wearable devices because it is difficult to remove once they occur (H. Halvaei, L. Sornmo, and M. Stridh, Signal quality assessment of a novel ECG electrode for motion artifact reduction, Sensors, 2021, 21, 5548.).

Electrocardiographs for measure ECGs in a comfortable lying and resting state with wet electrodes attached to the skin to be fixed. Wearable electrocardiographs must be used with dry electrodes contacting with preset body parts in an unfixed and moving posture. Thus, motion artifact noise is more likely to occur from wearable devices. It is important to reduce motion artifacts for accurate and comfortable ECG readings. However, the four electrodes installed in the wearable device are inevitably affected by motion artifacts.

Motion artifacts are an important cause of baseline wandering (L. Zhong, Pub. No.: US2014/0023134A1, Pub. Date: Jan. 23, 2014). The baseline wandering in the electrocardiogram may occur as a cause of human body motion. Baseline wandering noise is just as prone to wearable devices as motion artifacts. It is also difficult to remove baseline wandering noise in wearable devices, and reducing baseline wandering noise is important.

Motion artifacts are analyzed in order to prepare a criterion for determining which embodiment of the various embodiments described so far, is advantageous for a wearable device. FIG. 35 is an electrical equivalent circuit model in which motion artifacts voltage sources are inserted into the electrical equivalent circuit model of FIG. 25. Each independent motion artifacts voltage source is connected to one electrode. The motion artifacts voltage source is connected in series with the electrode resistance or electrode impedance. In general, the higher the electrode resistance, the stronger the motion artifacts. Analyzing FIG. 35, the following result is obtained. For convenience, power line interference is omitted.

$${v}_{1} = - {v}_{a} + {v}_{m 1} - {v}_{m 4} … … … … … … … … … … … … \qquad ( Equation 21 )$$

$${v}_{2} = {v}_{b} + {v}_{m 2} - {v}_{m 4} … … … … … … … … … … … … \qquad ( Equation 22 )$$

Naturally, each motion artifacts is applied to each electrode. Thus, vₘ₁ is applied to v₁ and vₘ₂ is applied to v₂. However, it should be noted that −vₘ₄ generated from the input electrode of the electrode driver is commonly applied to the two measured electrocardiogram voltages v₁ and v₂.

The reason for obtaining this result is that the voltage of the electrode driver input electrode becomes substantially zero even if there is a motion artifact or baseline wandering. That is, this is because the electrode driver input electrode becomes a virtual ground. This result means that the motion artifacts generated from the electrode driver input electrode are induced to the ECG measuring electrode.

The above results are important. In particular, considering that the electrode driver input electrode is a virtual ground, it is very surprising that the motion artifacts generated from the virtual ground electrode are transferred to all measurement electrodes. The above expressions mean the following. The following will be referred to as considerations or rules regarding motion artifacts.

Motion artifacts generated from the electrode driver input electrode are transferred to all ECG measurement electrodes. When using four electrodes, it propagates to two measurement amplifiers, and when using additional chest electrodes, it propagates to the chest electrodes as well. Therefore, it is important to reduce the motion artifacts generated from the electrode driver input electrode. It is a natural and well-known fact that electrodes for measuring ECG leads should be installed so that motion artifacts or baseline fluctuations are less likely to occur. However, it is more necessary to consider installing not only the ECG lead measurement electrode but also the input electrode of the electrode driver at a location where motion artifact or baseline fluctuation is weak.

Another surprising result is that vₘ₃ does not appear in common to v₁ and v₂. Motion artifacts generated from the electrode driver output electrode do not propagate to any ECG measuring electrode. Therefore, it can be concluded that it is advantageous to place the electrode driver output electrode at a location where motion artifacts are expected to be high or strong.

It can be seen from i) above that two motion artifacts are applied to one ECG lead signal amplifier. The first motion artifact is generated from an electrode connected to measure an electrocardiogram signal. The second motion artifact is generated at the electrode to which the electrode drive input is connected. This is a very important consideration that must be taken into account in embodiments implementing the present invention.

According to the present invention, after measuring and obtaining two ECG lead signals, four ECG lead signals are calculated and obtained. Motion artifacts included in the two ECG lead signals are also mixed with the calculated four ECG lead signals. Therefore, it is important to measure the two ECG lead signals so that motion artifacts do not occur.

As described many times before, the embodiments of the present invention use a single-ended input amplifier. For reference, FIG. 37 will be analyzed for the case of using a differential amplifier. When the voltage between electrode RA and electrode LA is amplified using a differential amplifier, the following result is obtained.

$$V ( RA ) - V ( LA ) = - {v}_{a} + {v}_{m 1} - {v}_{m 4} = {v}_{1} … … … … … … … \qquad ( Equation 23 )$$

In other words, even if a differential amplifier is used, the result of motion artifacts is the same as when using a single-ended input amplifier. Therefore, there is a further need to use a single-ended input amplifier in the present invention.

This gives us important considerations or rules on how to respond to motion artifacts. It is important to install ECG electrodes in consideration of which function is to be assigned to each ECG electrode. It is very important to carefully consider the electrode arrangement that is advantageous to motion artifact and baseline fluctuation among the various embodiments from Embodiment Groups A to D described above, and select embodiments accordingly. That is, in order to implement an electrocardiogram measuring device with less noise, the above considerations regarding motion artifacts must be applied in addition to the considerations regarding the electrode arrangement of the electrode driver described above.

In order to implement an electrocardiogram measurement device with low noise, the shape and installation location of each electrocardiogram electrode should be considered. Also, it should be considered which component is to be connected to each ECG electrode. For this purpose, a suitable shape for the wearable device should be designed. In the present invention described so far, when the human body part contacted by the electrode driver input electrode is determined, electrodes for electrocardiogram measurement are automatically assigned to the remaining two human body parts. Therefore, it is convenient to give priority to the electrode driver input electrode in the assignment of electrodes. From now on, the electrode driver input electrode will be considered preferentially when presenting the preferred embodiments in turn for specific shapes such as a ring, watch, clip shape, etc.

Using FIG. 24 and the like above, it has been described that an electrocardiogram measuring device having four electrodes can be implemented in the form of a ring worn on one finger. A preferred first embodiment for reducing motion artifacts in an electrocardiograph having four electrodes included in a ring will now be described. In the first embodiment, two electrodes can be placed on the inner surface of the ring. The skin of the finger contacting the inner surface of the ring is soft, has low electrode resistance, and contains moisture. Therefore, electrodes contacting the skin generate less motion artifacts. The electrode that generates less motion artifact should be selected as an electrode driver input electrode.

The two electrodes installed on the inner surface of the ring are set as an electrode driver input electrode and output electrode, respectively. This corresponds to the electrical connection shown in FIG. 24 for embodiment A2. In order to reduce motion artifacts, the two electrodes installed on the inner surface of the ring need to be larger than electrodes 111 and 114 shown in FIG. 17.

The remaining two electrodes are connected to two amplifiers for electrocardiogram measurement. The remaining two electrodes are installed on the outer surface of the ring and contact a finger of the other hand and the left leg, respectively. It is also advantageous to reduce motion artifacts if the electrodes 112 and 113 of FIG. 17 installed on the outer surface are wide.

In addition to the above-described embodiment, an embodiment in which two electrodes are installed on the inner surface of the ring and each is used as an output of an electrode driver and one electrocardiogram sensing electrode is also possible. In this case, the two electrodes installed on the upper and lower surfaces of the ring are used as the input electrode of the electrode driver and another sensing electrode for an ECG signal. These correspond to embodiment B3 and embodiment B4, respectively. In the case of the ring, these embodiments are generally considered not superior to embodiment A2 above from the point of view of motion artifacts. However, it is possible to implement these embodiments depending on the design of a ring.

On the other hand, it is necessary to install a photoplethysmograph along with an electrocardiogram a ring. As described above, a photoplethysmograph can generate an alarm to the user when an arrhythmia occurs by analyzing photoplethysmogram signal. The photoplethysmograph should be mounted on the inner surface of the ring to contact a finger. Therefore, there may be insufficient space to install two ECG electrodes on the inner surface of a ring. In addition, installing one electrode with a large area on the inner surface of the ring and using this electrode as the input electrode of the electrode driver is advantageous in reducing motion artifacts.

In this case, three electrodes can be placed on the outer surface of a ring. The electrical connection of an embodiment in which three electrodes are placed on the outer surface of a ring is shown in FIG. 36. FIG. 36 shows a second preferred embodiment for a ring. For an embodiment, when the embodiment of FIG. 36 is implemented as a ring, the electrode driver input electrode LA may be disposed on the inner surface of the ring worn on a finger of the left hand, and three electrodes may be disposed on the outer surface of the ring.

FIG. 37 is an electrical equivalent circuit model of the embodiment of FIG. 36. Analyzing this model, the following results can be obtained. For convenience, power line interference is omitted.

$${v}_{1} = - {v}_{a} + {v}_{m 1} - {v}_{m 4} … … … … … … … … … … … … … \qquad ( Equation 24 )$$

$${v}_{2} = {v}_{b} + {v}_{m 2} - {v}_{m 4} … … … … … … … … … … … … … \qquad ( Equation 25 )$$

The above equations are the same as the analysis results of FIG. 35 which is for FIG. 24. This is because the position of the electrode driver input electrode is the same. In this embodiment, since the area of the electrode driver input electrode is increased, motion artifacts −vₘ₄ of the electrode driver input electrode, which is commonly applied to the electrocardiogram voltage v₁ of the right hand electrode and the electrocardiogram voltage v₂ of the left leg electrode, can be reduced.

In the above embodiment, when the ring is worn on a finger of the left hand, a finger of the right hand contacts two electrodes RA and DRA, and one electrode LL contacts the left leg. In this case, two electrodes may be installed on the upper surface of the ring as shown in FIG. 38.

In the present invention, four electrodes are brought into contact with three human body parts. If a user wears a ring or watch on the user's left hand, the user places the left hand on the left leg when taking the ECG. Therefore, in this case, the most unstable part among the three body parts is the right hand. Therefore, if the right hand can be stabilized, the motion artifacts generated from the right hand can be reduced. In the case of using the electrocardiograph included in the ring of FIG. 38, if the right hand is placed on the left hand or left leg and one right hand finger is placed on the two electrodes 3810 and 3820, the right hand can be stabilized and motion artifacts can be reduced.

Meanwhile, another embodiment in which one electrode is installed on the inner surface of a ring is shown in FIG. 39. In this embodiment, a ring is implemented so that the finger of the right hand contacts one electrode 3910 (RA electrode), and the left leg contacts two electrodes 3920 and 3930 (LL electrode and DLL electrode). In this embodiment, the electrodes 3940 and 3950 inside the ring are connected as one and assigned as an electrode driver input electrode. When measuring the electrocardiogram, the two electrodes 3920 and 3930 are also advantageous for stably contacting the left leg. Also, the area of the right hand electrode 3910 can be increased. Therefore, this embodiment is advantageous for motion artifact reduction. In this case, the lower part of the ring may be made thicker, wider, and mechanically stronger than a conventional ring.

The embodiments described above, preferred for reducing motion artifacts in the electrocardiogram included in a ring, have in common that the electrode driver input electrode is installed on the inner surface of the ring.

So far, it has been described that a single-ended input amplifier is used to implement the present invention. However, as previously described, the present invention involves using a differential amplifier instead of a single-ended input amplifier. As described above, it is not necessary to use a differential amplifier in the present invention. Using a single-ended input amplifier is more advantageous than using a differential amplifier in terms of size, cost, and power consumption. However, it is possible to use a differential amplifier instead of a single-ended input amplifier to implement the present invention. In the present invention, all embodiments using single-ended input amplifiers can be replaced with embodiments using differential amplifiers as they are. Therefore, even if the differential amplifier is not specifically mentioned or illustrated, the present invention includes embodiments using the differential amplifiers.

The method of using differential amplifiers in the present invention is as follows.

Two differential amplifiers are used instead of two single-ended input amplifiers used in the present invention.

Among the two input terminals of the differential amplifier, one is connected to the input terminal of the single-ended input amplifier, and the other is connected to the input of the electrode driver. Since two single-ended input amplifiers are used in the present invention, a method for connecting two differential amplifiers is determined. The polarity of the input terminals of the differential amplifier is not critical. This is because the measured value can be treated according to its polarity.

One electrode driver is used in the present invention and no Wilson Central Terminal is used. Even when using a differential amplifier, the matters related to the electrode driver are the same as when using a single-ended input amplifier. The input of the electrode driver connects to one electrode.

The output of the electrode driver is connected to one electrode.

Since the present invention aims at a wearable device and obtains six limb leads, four electrodes contacting three human body parts are used. These statements do not change when using a differential amplifier instead of a single-ended input amplifier.

Therefore, in the case of using the differential amplifiers in this embodiment, the two differential amplifiers in common have one input terminal connected to the electrode installed on the inner surface of a ring.

It can be seen through circuit analysis that even when a differential amplifier is used the motion artifacts are the same as when a single-ended input amplifier is used. This is another reason to use a single-ended input amplifier in the present invention.

In the previous three electrode embodiments, it was described that it may be necessary to measure only lead I depending on the situation or necessity. It is possible to measure only lead I without contacting the left leg electrode, using the embodiments of FIG. 24 and FIG. 38. In the embodiments of FIG. 24 and FIG. 38, power line interference can be reduced without contacting the left-leg electrode. The embodiments of FIG. 26 and FIG. 39 are disadvantageous when only lead I is to be measured. FIG. 26 and FIG. 39 cannot reduce power line interference unless the left leg electrode is brought into contact with the left leg.

A photoplethysmograph installed on the inner surface of a ring that touches a finger analyzes the photoplethysmogram signal and instructs an alarm to occur when it is determined that there is an abnormality. The ring can transmit an instruction signal for alarm generation to a smart device or a smartphone wirelessly connected to the ring.

Now, embodiments in which four electrocardiogram electrodes are installed on a watch body and a watch band will be described. There are three preferred embodiments in this case.

The first preferred embodiment is as follows. The most preferred location for installing the input electrode of the electrode driver is the bottom surface of the watch body. This is because the bottom surface of the watch is always in contact with the user's wrist and provides the widest space for placing the ECG electrodes. Also, since the skin on the wrist that the bottom of the watch touches is soft and moist, there are less motion artifacts. Therefore, in FIG. 16, the two electrodes of electrode 1610 and electrode 1620 installed on the bottom of the watch body are assigned to be the input electrode and output electrode of the electrode driver, respectively. This corresponds to the electrical connection shown in FIG. 24 for embodiment A2. This embodiment is very similar to the first preferred embodiment of the ring described above.

The remaining two electrodes are connected to two amplifiers for electrocardiogram measurement. Among the remaining two electrodes, one electrode 1640 in FIG. 16 is installed on the main body of the watch to make contact with a finger of the other hand. The other electrode 112 of FIG. 16 is installed on the outer surface of the watchband to contact the left leg.

Another embodiment in which two electrodes are installed on the bottom of the watch body is considered. An embodiment in which the electrode 1640 in FIG. 16 installed on the watch body is selected as an electrode driver input, and the electrodes 1610 or 1620 installed on the bottom of the watch is selected as an electrode driver output can be considered. However, in this embodiment, since the unstable right hand contacts the small electrode 1640 of FIG. 16, motion artifacts are relatively large. Therefore, this embodiment is not a good embodiment.

On the other hand, in the first embodiment, which is preferred when implementing an electrocardiograph included in the watch described above, the right hand electrode (RA), which has the smallest electrode size and is in contact with the right hand which is an unstable body part, is used as an ECG measurement electrode. Therefore, motion artifacts may occur in the RA electrode of this embodiment. Therefore, in the present embodiment, a method of stabilizing a watch worn on one hand with the other hand is devised in order to reduce motion artifacts.

One embodiment of stabilizing a watch worn on one hand with the other hand to reduce motion artifacts is described using FIG. 16. The watch is stabilized by simultaneously holding one side of the band and the electrode 113 with the thumb and a finger of the other hand. Electrode 113 is connected to the electrode driver input. Electrode 1610 on the bottom of the watch body is connected to the electrode driver output. When the watch is worn on the left hand, another electrode 1620 on the bottom of the watch body is connected to an electrocardiogram measurement amplifier to measure Lead I. The electrocardiogram measured by the electrode 1620 on the bottom of the watch body has the advantage of being less affected by motion artifacts. The electrode 112 installed on the outer surface of the watchband is in contact with the left leg. This corresponds to embodiment B3. This embodiment is one of the preferred embodiments for installing an electrocardiograph included in a watch. In this embodiment, an electrode facing electrode 113 may be additionally installed on a band facing the band on which electrode 113 is installed. The above electrode and electrode 113 may be held together with the other hand.

The above two embodiments can be expressed as follows: An electrocardiogram measuring device, characterized in that input of an electrode driver is connected to an electrode installed on the bottom of the watch body or the outer surface of the watch band, and another electrode installed on the watch band contacts the left leg.

Another structure of a watch suitable for reducing motion artifacts generated from the right hand electrode is shown in FIG. 40. The embodiment of FIG. 40 is the third preferred embodiment for installing an electrocardiograph included in a watch. When the watch of FIG. 40 is worn on the left hand, if the two electrodes 4010 and 4020 are held with the thumb and a finger of the right hand, respectively, the watch is fixed to the right hand, thereby reducing motion artifacts.

The two electrodes 4010 and 4020, which are held by the thumb and a finger of the right hand, are assigned as the electrode driver output electrode and the electrocardiogram measurement electrode, respectively. This embodiment electrically corresponds to the embodiment of FIG. 36. That is, this corresponds to embodiment B1. In this embodiment, since the electrode driver input electrode is installed on the bottom of the watch body, less motion artifacts is generated. This embodiment may be preferred.

In this embodiment, the electrode 4030 of FIG. 40 installed on the bottom of the watch body contacts the left hand wearing the watch, and the electrode 4050 installed on the outer surface 4080 of the watch band contacts the left leg. In order to reduce motion artifact, this embodiment satisfies the electrode use rule of using the right hand electrode (DRA), which is the smallest in size and has the most unstable body part in contact with the electrode, as an electrode driver output.

Other embodiments can be applied to the watch of FIG. 40. Electrically connect the two electrodes 4010 and 4020 held by the thumb and a finger of the right hand and assign them to the electrode driver input. Among the two electrodes on the bottom of the watch body, one is assigned as an electrode driver output electrode and the other is assigned as an ECG measurement electrode. Electrodes installed on the outer surface of the watch band become ECG measurement electrodes that are in contact with the left leg. Then, lead I and lead II are measured at each electrocardiogram lead measurement electrode. This embodiment is also a good embodiment for motion artifacts.

The two embodiments using the watch of FIG. 40 can be expressed as follows. An electrocardiogram measuring device, wherein an input electrode of an electrode driver is assigned to at least one of two electrodes installed on a watch body so that the watch can be stabilized with the other hand not wearing the watch, and another electrode installed on the outer surface of the watch band contacts with left leg.

Thus, the superiority of the embodiment can be judged by motion artifacts analysis. Embodiments A2 and B1, which are preferred for reducing motion artifacts in the electrocardiograph included in a watch, have in common that an electrode driver input electrode is installed on the bottom of the watch body.

The following thoughts about the electrocardiograph included in a ring described above are also applied to an electrocardiograph included in a watch. It has been described above that it may be necessary to measure only lead I depending on the situation or necessity. Embodiment A2 in FIG. 24, B3, and B1 in FIG. 36, are advantageous when only lead I is to be measured without contacting the left leg. In the above three embodiments, A2, B3, and B1, power line interference can be reduced without contacting the left leg. For reference, the embodiments of FIG. 26 and FIG. 28 are disadvantageous when only Lead I is to be measured. In the embodiments of FIG. 26 and FIG. 28, power line interference cannot be reduced unless the left leg electrode is brought into contact with the left leg.

A photoplethysmograph installed on the bottom of the watch may analyze a photoplethysmogram signal and generate an alarm for a user.

As described above with FIGS. 18 and 26, an electrocardiogram measuring device can be implemented by being coupled to the pants so that the electrocardiogram can be measured immediately when the electrocardiogram is to be measured. Two clips serving as two electrodes are used to attach the electrocardiogram measuring device to the inside of the pants, that is, between the pants and the user's left lower abdomen. When the electrocardiogram measuring device is attached to the left lower abdomen of the pants using two clips, the two electrodes and a photoplethysmograph automatically contact the left lower abdomen of the user. When the photoplethysmograph sends an alarm or the user wants to measure the electrocardiogram, the user touches a finger of the left hand to the first clip and a finger of the right hand to the second clip.

The electrocardiogram measuring device corresponds to embodiment A3 in which two electrodes are in contact with the left lower abdomen. Since the two electrodes contacting the left lower abdomen are in contact with soft and moist skin, it is suitable to assign one electrode as the electrode driver input electrode. Since the two electrodes are adjacent and contact the same body part, the other electrode is assigned as the electrode driver output electrode.

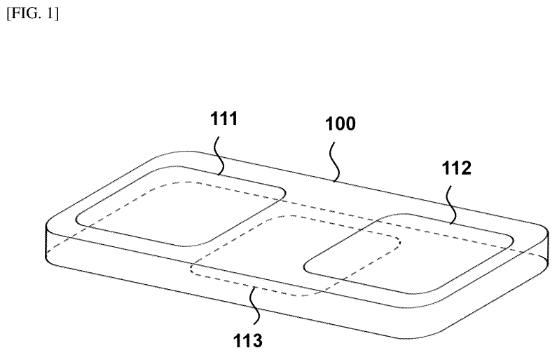

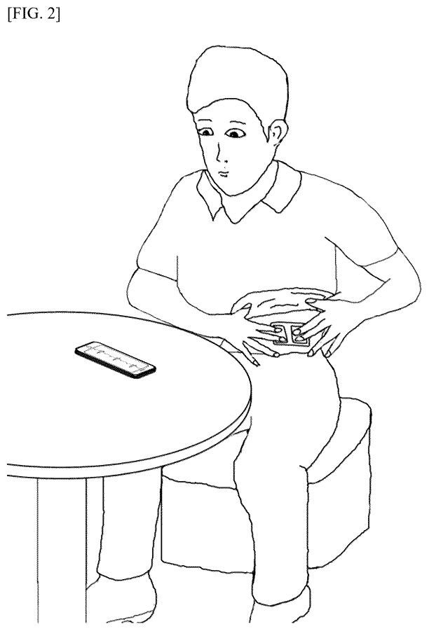

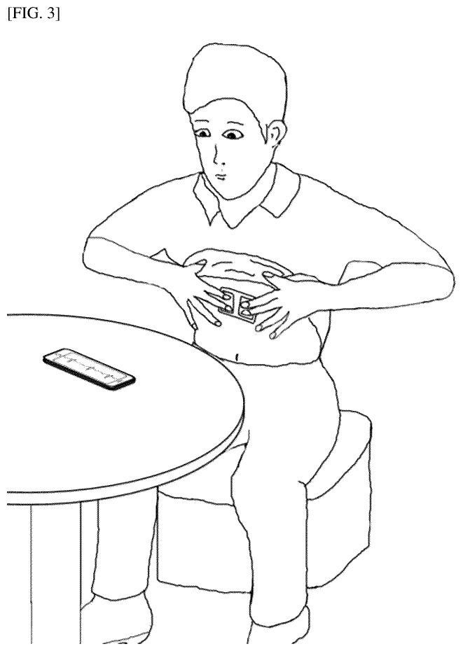

The electrocardiogram measuring device shown in FIG. 1 described as plate-shaped above is flat and has a length longer than the width. Any one of the three electrodes installed in the electrocardiogram measuring device may be divided into two so that the device has four electrodes. That is, one electrode of the three can be made smaller and one electrode can be added nearby. Electrode 113 in FIG. 1 is preferably divided into two electrodes, and the two electrodes are brought into contact with the left leg or left lower abdomen to reduce motion artifacts.

Nanoelectrodes can be used to reduce motion artifacts. The electrode impedance of nanoelectrodes is low and the nanoelectrodes are made of materials other than conventional metals (S. Yao, and Y. Zhu, Nanomaterial-Enabled Electrodes for Electrophysiological Sensing: A Review, The Journal of The Minerals, Metals & Materials Society, Vol. 68, pp. 1145-1155, 2016)

In order to reduce the electrical contact resistance of the nanoelectrode and prevent it from being broken to enhance mechanical strength, a nanoelectrode and a metal electrode may be combined and used. In this embodiment, the nanoelectrode and the metal electrode are bonded well and the two objects are bonded using an adhesive material having low electrical resistance.

Digital signal processing may be employed in all embodiments according to the present invention. That is, after converting a voltage amplified by an amplifier into a digital signal, necessary operations can be performed by a microcontroller, and the result of the operation can be output through the DA converter. The output of the DA converter can be applied to the input of the electrode driver. That is, in the embodiments of the present invention, the input of the electrode driver can be received from a DA converter.

However, since the ECG signal is weak, to apply digital signal processing, voltage amplification using an amplifier is essential. AD conversion with appropriate resolution can be performed only after the electrocardiogram signal is amplified. An electrocardiogram signal received from one electrode is amplified, AD converted, digital signal processed, and the output of the DA converter is applied to an electrode driver input.

In the analysis of all embodiments, it was assumed that the output impedance of the electrode driver was low. However, a series resistor may be inserted at the output of the electrode driver to prevent the electrode driver from applying excessive current to the human body.

As described in the three electrode embodiments, when the electrodes are brought into contact with human body parts, an electrocardiogram measuring device can be automatically powered on by a current sensor. A user can also start taking electrocardiograms with the current sensor. Therefore, if a current sensor is used, a mechanical switch may not be used. Details follow the three electrode embodiments.

The present invention discloses an apparatus for obtaining a multi-channel ECG lead signal while reducing noise such as power line interference, motion artifacts, and baseline wandering. The present invention includes a method for reducing power line interference, motion artifacts, baseline wandering, and high-frequency noise by deep learning. In particular, since power line interference is a signal having a constant frequency, it is easy to produce training data, and training is also easy, and the result is effective. In addition, motion artifacts, baseline wandering, and high-frequency noise can be removed by using a deep learning method to create and train training data.

An embodiment of measuring MCL in the above three electrode embodiments has been described. Even in the embodiments using four electrodes, embodiment A2 in FIG. 24, embodiment B1 in FIG. 36, and embodiment B6 are suitable for measuring the chest lead with MCL based on the LA voltage. Alternatively, the chest lead can be measured relative to the Wilson Central Terminal using five electrodes.

Although the electrocardiogram measuring device has been described as including four electrodes in the previous embodiments of the electrocardiogram measuring device, another embodiment of the electrocardiogram measuring device according to the present invention may include five electrodes. The operation principle of the electrocardiogram measuring device according to the present invention including the five electrodes is the same as the previous description for the embodiments including the four electrodes. An important point is that the electrocardiogram measuring device including five electrodes according to the present invention includes three amplifiers that respectively receive three electrocardiogram signals from the three electrodes, and each of the three amplifiers amplifies one electrocardiogram signal so that the device simultaneously amplifies three electrocardiogram signals. Thus, three electrocardiogram signals are actually measured simultaneously.

An electrocardiogram measuring device including the five electrodes can be easily implemented as described above. The method of using the electrocardiogram measuring device including the five electrodes according to the present invention is almost the same as the method of using the electrocardiogram measuring device according to the present invention including the four electrodes.

The three ECG signals measured by the electrocardiogram measuring device including five electrodes according to the present invention include, for an embodiment, two limb leads and one chest lead. Alternatively, the three electrocardiogram signals may be one limb lead and two chest leads. The electrocardiogram measurement device according to the present invention including the five electrodes may be implemented in a form suitable for the use method and function.

The electrocardiogram measuring device including four electrodes described above or the electrocardiogram measuring device including five electrodes described so far can obtain the standard 12-lead electrocardiogram after obtaining two limb leads and one chest lead by calculation. To this end, a method similar to that of Dower et al. (U.S. Pat. No. 4,850,370, Patent Date: Jul. 25, 1989) or a method by deep learning may be used.

One electrocardiogram measuring device installed on a chest strap including at least four electrodes can be used to obtain seven or more electrocardiogram lead signals. An electrocardiogram measuring device installed on a chest strap may be used in parallel with an electrocardiograph included in a watch, an embodiment may use Hwang (Hwang, WO2023/018318A1, Pub. Date: Feb. 16, 2023). In this case, the standard 12-lead electrocardiogram can be generated by using a method similar to Dower et al. or by using a deep learning method.

Embodiments from now on will be referred to as Embodiment Group F. Embodiment Group F belongs to the embodiments using three electrodes described above. An embodiment of Embodiments Group F is as follows.

An electrocardiogram measuring device, comprising:
a first electrode and a second electrode configured to receive a first ECG voltage of a first body part and a second ECG voltage of a second body part in contact therewith, respectively;
a first amplifier configured to receive the first ECG voltage from the first electrode;
one electrode driver configured to receive the second ECG voltage from the second electrode and transmit the second ECG voltage to a third electrode and a second amplifier;
the third electrode configured to receive the second ECG voltage from the electrode driver and transmit the second ECG voltage to a third body part in contact therewith;
the second amplifier configured to receive the second ECG voltage from the electrode driver;
an AD converter connected to an output terminal of each of the two amplifiers to convert output signals of the two amplifiers into two digital signals,
wherein the two amplifiers each receive and amplify one ECG voltage simultaneously.

So far, embodiments of obtaining six limb lead signals using four electrodes have been disclosed. Using four electrodes and an electrode driver is advantageous to reduce power line interference. However, when space for installing four electrodes is insufficient, using an embodiment using three electrodes is necessary. That is, when trying to manufacture a wearable electrocardiogram a small size, it may be cumbersome to install four electrodes. For wearable electrocardiographs, power line interference generally occurs less than for electrocardiogram hospital operating rooms, etc., so it is possible to obtain reduced power line interference while using three electrodes.

FIG. 41 shows an embodiment in which six ECG limb lead signals are obtained using three electrodes. In Embodiment Group F, the output of the electrode driver connected to one electrode in contact with a human body part is connected to the input terminal of one single-ended input amplifier. The embodiment of FIG. 41 can be seen as combining the two electrodes RA and DRA of the embodiment of FIG. 36 into one electrode. The Embodiment Group F has a feature that the output voltage of the electrode driver is one electrocardiogram lead signal. The output of an electrode driver is an electrocardiogram lead signal is a notable feature. Another ECG lead signal is received from an electrode not connected to the input or output of the electrode driver and amplified by another amplifier 4120.

Embodiment Group F simultaneously measures two ECG signals, as in the above-described embodiments using three electrodes. Embodiment Group F looks similar to the embodiment using three electrodes and a band pass filter described in FIG. 4. However, the frequency band of the electrode driver used in Embodiment Group F includes the power line interference frequency and the frequency band of the electrocardiogram signal according to ANSI 60601-2-47, that is, from 0.67 Hz to 40 Hz. If the frequency band of the electrode driver includes 0.67 Hz or less, an additional effect of reducing baseline wandering can be obtained.

FIG. 42 is an electrical equivalent circuit model of FIG. 41 for analyzing the electrical characteristics of FIG. 42. The input voltage v1 of the single-ended input amplifier 4110 and the input voltage v2 of the single-ended input amplifier 4120 are expressed as follows.

$${v}_{1} = - {v}_{a} + {v}_{m 1} - {v}_{m 3} - i n {R}_{e 1} … … … … … … … … … … … … \qquad ( Equation 26 )$$

$${v}_{2} = {V}_{b} + {v}_{m 2} - {v}_{m 3} + in * {R}_{e 1} / ( H ( f ) + 1 ) … … … … … … … … … \qquad ( Equation 27 )$$

Looking at the above two analyzed results, the following conclusions are obtained.

The two measured electrocardiogram voltages commonly include motion artifact vₘ₃ of the electrode connected to the input of the electrode driver. This result is the same as that of the embodiments using four electrodes. The inclusion of the motion artifact from an electrode connected to the electrode driver input terminal is common. Thus, when measuring two ECG lead signals using negative feedback with three ECG electrodes and an electrode driver, it is necessary to reduce motion artifacts generated from an electrode connected to the electrode driver input terminal. Also, in this embodiment, two motion artifacts are mixed with one ECG lead signal.

The power line interference of v₂ in FIG. 42 is reduced to 1/(H(f)+1). This is because, as shown in FIG. 42, substantially all of the power line interference current flows to the output terminal of the electrode driver 4130, and the output impedance of the electrode driver 4130 is very small. The effect of removing power line interference is the same as that of the above-described embodiment using three electrodes and a band-pass filter. However, this embodiment is easier to implement than the embodiment using a band-pass filter. The effect of removing power line interference in this embodiment is the same as when using four electrodes. However, it should be noted that the power line interference in v₁ is not reduced.

It should be noted that v₁ contains unamplified ECG voltage vₐ. This is because amplifier 4130 acts as a negative feedback amplifier. Therefore, amplifier 4110 must be included to obtain amplified vₐ. In order to sense an ECG voltage in an electrocardiogram measuring device, amplification is essential. This is because the voltage of a normal ECG signal is very small. The magnitude of the R wave, the largest peak in the ECG signal, is usually very small, about one mV.

Because the amplifier 4130 outputs v1 to the electrode RA, it is proper to call the amplifier 4130 an electrode driver. The input of the amplifier 4130 is the ECG voltage induced at the electrode LA. Thus, the amplifier 4110 receives the ECG voltage induced at the electrode LA

Another consideration for this embodiment is as follows. Motion artifact analysis using FIG. 37 was previously performed. Looking closely at this analysis result, one electrode driver was used in FIG. 37, but the electrode driver is not helpful for reducing motion artifacts. The electrode driver. Is used to reduce power line interference. Therefore, it may be advantageous to use three electrodes for motion artifact reduction. For example, in FIG. 38 using four electrodes, when the electrode 3810 and the electrode 3820 are combined to form one electrode, the area of the combined electrode is widened. Accordingly, an area in contact with a finger of the right hand can be widened, and then motion artifact can be reduced. This may be an important conclusion.

Embodiment Group F has six embodiments shown in Table 5 in FIG. 43. Detailed descriptions are omitted. To select one of Embodiment Group F for a wearable electrocardiograph, it should be considered that an electrode driver input should be connected to an electrode with low motion artifacts. This is the same teaching in the embodiments using four electrodes. In the embodiment of FIG. 41, the electrode driver input electrode is brought into contact with the left hand. The embodiment of FIG. 41 is suitable for implementation as a watch or a ring. As in the above-described embodiment using four ECG electrodes, the input of the electrode driver may be connected to the electrode installed on the bottom of a watch body or the inner surface of a ring. In an embodiment of the device using two clips, the electrode driver input is connected to the left-leg electrode LL.

Embodiments of the electrocardiogram measuring device described above may include three or four electrocardiogram measuring electrodes. However, a common purpose pursued by all the embodiments described above is to obtain six ECG lead signals from the electrocardiogram measuring device. It has been described that the number of electrocardiogram lead signals to be measured and obtained in order to achieve this object is two. In all embodiments including three or four electrocardiogram measuring electrodes described above, if two electrocardiogram lead signals are measured and obtained, four electrocardiogram lead signals can be additionally obtained. That is, measuring and obtaining three ECG lead signals is an unnecessary, inefficient, and redundant method and falls within the scope of the present invention. In all the embodiments described above, because a single-ended input amplifier is used, one amplifier output is characterized by one ECG lead signal.

Compared to the embodiment including three ECG electrodes, in the embodiment including four ECG electrodes described above, one additional ECG electrode is connected to an input of one electrode driver. Other than this, the embodiments including four ECG electrodes and the embodiments including three ECG electrodes are identical in many respects.

All the embodiments described above are electrocardiogram measuring devices including at least three electrocardiogram electrodes in contact with three body parts of the right arm (RA), left arm (LA), and left leg (LL).

In general, in relation to ECG, “channel” and “lead” are used interchangeably, and they mean one ECG signal or ECG voltage. In addition, it can be expressed in various ways according to circumstances and needs, such as an electrocardiogram channel, an electrocardiogram lead, an electrocardiogram lead signal, or an electrocardiogram signal voltage.

In order to additionally obtain four ECG lead signals, requirements for the two ECG lead signals must be clarified. That is, the requirements for an electrocardiogram measuring device for measuring two electrocardiogram lead signals must be clarified. Hereinafter, requirements for an electrocardiogram measuring device including three or four electrocardiogram measuring electrodes in order to measure at least two electrocardiogram lead signals will be described.

Partial contents of the embodiments described from now on are based on WO2023/018318A1 (international application date: Aug. 16, 2022, inventor: HWANG, In-Duk). In addition, the embodiments described from now on can apply the contents of the above application without overlapping descriptions.

An electrocardiogram measuring device that presets parameters representing the arm to be worn

One of the requirements for an electrocardiogram measuring device including three or four electrocardiogram measuring electrodes is as follows. It must be determined what the two electrocardiogram lead signals obtained by measurement are. This will be described with an example.

In the embodiment of the watch shown in FIG. 16, when the watch is worn on the left hand, the electrodes 1610 and 1620 installed on the bottom of the watch body contact the left wrist. The electrode 1640 installed on the right side of the watch body contacts one right-hand finger. Electrodes 112 installed on the watch band contacts the left leg. According to the connection and configuration of the four electrodes and the electrocardiogram measurement circuit installed inside the watch, this watch can be implemented as embodiments of A2 in FIG. 24, B4, B3 in FIG. 28, C4, C5, C6 in FIG. 30, and D2 in FIG. 33. Naturally, the two ECG lead signals measured in each of the above seven embodiments are different from each other.

FIG. 44 shows the watch worn on the left hand and the watch worn on the right hand. When the watch is worn on the left hand, the electrode 1640 is installed on the right side of the watch body. When the watch is worn on the right hand, the electrode 1640 seems to be installed on the left side of the watch body. This is because the watch worn on the right hand has rotated the same watch itself worn on the left hand counterclockwise about 150 degrees around an axis perpendicular to the figure. The two watches in FIG. 44 are the same watch. However, the display or screen shown on the watch worn on the right hand is the display shown on the watch worn on the left hand rotated 180 degrees around the axis perpendicular to the FIG. 44. Again, the two watches in FIG. 44 are the same watch. More on this later.

When the watch is worn on the right hand, electrodes 1610 and 1620 installed on the bottom of the watch body contact the right wrist, and electrode 1640 contacts a finger of the left hand. The electrode 112 contacting the left leg contacts the left leg whether the watch is worn on the right hand or worn on the left hand. The connection and configuration of the four electrodes and the electrocardiogram measurement circuit installed inside the watch are the same as that of the watch worn on the left hand. When the watch is worn on the right hand, the watch may correspond to seven embodiments of A1, B1 of FIG. 36, B2, C1, C2, C3, and D1.

The seven embodiments in which the watch is worn on the left hand and the seven embodiments in which the watch is worn on the right hand each have a symmetrical relationship, and this relationship is shown in Table 6 of FIG. 45. In Table 6, the left and right embodiments are cases in which the same electrocardiogram measuring device, for example, a watch, is worn by changing hands. For example, A1 and A2 are cases in which the device of FIG. 24 is worn by swapping hands. What should be noted is that the left leg electrode 112 is still in contact with the left leg even when the watch is worn by changing hands.

When the watch is worn on the left hand and the electrocardiogram is measured, for example, in the embodiment of A2 of FIG. 24, the measured electrocardiogram lead signals are −lead I and lead III. However, when the watch is worn on the right hand, the ECG lead signals measured in embodiment A1 are Lead I and Lead II. That is, when the same watch is worn by changing hands, the two ECG lead signals measured by the watch are changed. Therefore, in order to know what the two ECG lead signals measured using the watch are, it is necessary to know which hand is wearing the watch.

This problem does not occur when the watch is worn only on one hand. Therefore, this embodiment includes the following to solve this problem: Two types of watches: a watch worn on the left hand only and a watch worn on the right hand only. This embodiment includes: a ring worn on the left hand only or a ring worn on the right hand only. The aforementioned problem does not occur in the long, flat, stick-shaped electrocardiogram measuring device described above unless it is used with the left and right sides switched.

An embodiment of the above watch worn on the left hand only includes: A watch comprising: one electrode in contact with the left hand; one electrode driver having an input connected to the electrode; and two amplifiers, wherein one of the two amplifiers, amplifies Lead III induced in an electrode contacting the left leg, wherein Lead II is calculated and obtained.

For a watch measures only one ECG lead signal, Lead I, Dusan has disclosed how the software analyzes the ECG lead signals measured by the watch and automatically determines which hand the watch was worn on to measure an ECG. (Sorin V. Dusan, Pat. No.: U.S. Pat. No. 10,045,708B2, Aug. 14, 2018) However, it is thought that the reliability of the method disclosed by Dusan is not high enough. This method may not be highly reliable.

The reason Dusan's method may not be highly reliable is as follows. First, it was assumed that FIGS. 3A, 3B, and 3C in Dusan are due to lead inversion, i.e., electrocardiograms measured by changing hands wearing the watch. However, while this assumption is true in many cases, it may be incorrect in some cases. A representative incorrect case is a case in which one electrocardiogram has Right Axis Deviation (RAD). When the electrocardiogram shows a RAD, the R-wave appears inverted. The cause of RAD may be right ventricular hypertrophy. That Dusan's assumption may be incorrect may be referred to the following three references.

Reference 1: Thomas B. Garcia, “12-Lead ECG, The Art of Interpretation”, 2nd Ed., Jones & Bartlett Learning, 2015, pp. 359-360.

Reference 2: Keith Wesley, “ECG AND 12-LEAD INTERPRETATION”, 5th Ed., Elsevier, 2017, p. 215.

Reference 3: James H. OKeefe, et al., “The Complete Guide to ECGs”, 4th Ed., Jones & Bartlett Learning, 2017, p. 578 and p. 581.

Second, Dusan compares the measured ECG with the ECG measured during the watch's enrollment phase. However, it may be difficult to compare the ECG measured in the case of an arrhythmia with the ECG measured in the enrollment phase.

Third, some users show lead I that the change in signal amplitude with respect to time is too small, that is, the ECG waveform is flat, making it difficult to perform any analysis. A case in which the change in magnitude of lead I is too small is when the direction of the electric axis of the heart is in the aVF direction, that is, downward. In this case, the change in magnitude over time is the largest in the aVF lead among the six limb leads. In this case, the waveform of lead I is flat. This phenomenon is a representative reason why electrocardiogram leads other than lead I is required. This is a very well-known fact in cardiology.

Therefore, the method disclosed by Dusan may be valid in many cases but may be erroneous in some cases. In medical diagnosis, not to make an incorrect diagnosis is more important than to make a correct diagnosis. Therefore, the validity of Dusan's method is lost. Dusan's method may present erroneous results to the user, which can be confusing and dangerous.

As described above, in the present invention, it is determined that Dusan's method is incomplete. Therefore, in order to exclude incompleteness, the present invention discloses the following method different from Dusan's method.

In the present invention, one electrocardiogram lead is additionally measured in addition to lead I. That is, lead III or lead II is additionally measured according to the wearing hand. However, in case a user's electrical axis of the heart is aVF, it is difficult to determine whether the additionally measured ECG lead is Lead III or Lead II by looking at only two ECG waveforms. In addition, it has already been explained that it is difficult to determine the hand wearing the watch using only lead I using Dusan's method. Therefore, although lead III or lead II is further measured in the present invention, in case a user's electric axis of the heart is in the aVF direction, it is determined that it is difficult to determine which hand the electrocardiogram measuring device was worn on and what the measured two ECG lead signals are, when a hand wearing a device is changed.

According to the above judgment, embodiments of the present invention include the following methods. In this method, a user pre-determines a hand to wear a wearable device, typically a watch or a ring. When a user uses a watch or ring for the first time, the device may be registered in a smartphone application.

The user stores the user's information on the watch or on the smartphone. In the registration step, two buttons for selecting a wearing hand are displayed on the screen of the smartphone or smartwatch, and the user selects one button. The two buttons indicate the left and right hands, respectively. When the user selects one button, the hand to be worn is set. When the hand to be worn is set, a parameter indicating the hand to be worn is stored in the watch or smartphone's memory.

A parameter representing the hand to be worn must be used to specify which two ECG leads are measured by performing the electrocardiogram measurement. For example, it should be determined whether the measured two electrocardiogram leads are (lead I and lead II), or (lead I and lead III). The above parameter is also used to select calculation formulas for obtaining additional electrocardiogram lead signals.

It has been previously described that the two watches shown in FIG. 44 are identical. One display or image of a watch can be obtained by rotating the other display or image of the other watch by 180 degrees. The display orientation is selected or changed by the user using the watch or smartphone application software. The selection of the display orientation is equivalent to the selection of a hand to wear the watch. Therefore, if the display orientation is set, it is unnecessary to set the hand on which the watch is worn to measure the electrocardiogram. That is, the step of selecting a hand to be worn using the two buttons described above is unnecessary. This is because the display orientation setting parameter can be used as the setting parameter of the hand wearing the watch in the watch application. Therefore, selecting the display orientation of the watch in the following embodiments is regarded as setting the hand wearing a watch.

Although the hand to be worn can be set in advance when a watch or ring is manufactured in a factory, a user must be able to check and change the hand to be worn finally.

After setting the wearing hand once, there is no need to set the wearing hand each time when measuring ECG lead signals. It can happen that a user measures ECGs wearing a watch on the opposite hand, without changing the setting. To check this has happened, the measured ECG leads and the ECG leads measured before may be compared to determine whether leads have changed or not. If the wearing hand has been changed, a pop-up window may ask the user to confirm the wearing hand. Electrocardiogram lead signals calculated using different calculation formulas may be modified according to the user's confirmation.

Embodiments according to the above description include: A watch measuring at least two electrocardiogram lead signals, including: at least three electrocardiogram electrodes; and a button for selecting a hand to wear, displayed on a screen of the watch.

Alternatively, one embodiment includes: A watch or a smartphone storing a parameter representing a hand wearing the watch in a memory.

Alternatively, one embodiment includes: An electrocardiogram measuring device that calculates and obtains additional electrocardiogram lead signals by selecting calculation formulas according to a selection of a button displayed on a watch display. The button is a button for selecting a hand to wear the watch or setting the display orientation of the watch.

Alternatively, one embodiment includes: An electrocardiogram measurement device that calculates and obtains additional electrocardiogram lead signals by selecting calculation formulas according to a selection of a button displayed on a smartphone screen. The button is for selecting a hand to wear the electrocardiogram measuring device.

Alternatively, one embodiment includes the following: An electrocardiogram measurement device that calculates and obtains additional electrocardiogram lead signals by setting a hand to be worn and selecting calculation formulas according to a pre-set parameter.

One embodiment includes the following.

An electrocardiogram measurement device comprising:
one electrode in contact with one hand;
one electrode driver having an input connected to the electrode;
one amplifier amplifying Lead II or Lead III induced to an electrode contacting the left leg; and
a smartphone that selects calculation formulas using a parameter representing a hand wearing the device and calculates and obtains lead III or lead II depending the parameter.

If the electrocardiogram measurement wearable device does not include a sufficiently wide display, a button for selecting a hand to be worn is displayed on a smartphone display, and calculation formulas for additionally obtaining electrocardiogram lead signals may be selected according to the selected button. The electrocardiogram measurement wearable device that does not include a wide display includes the devices described above along with the figures shown before, such as a ring and a device including two clips.

One embodiment according to the above includes: A smartphone displaying two buttons for selecting a hand to wear a ring on a smartphone screen, and selecting calculation formulas for obtaining additional electrocardiogram lead signals according to the selected button.

What has been described so far can be implemented in the steps of the flowchart of FIG. 46. Hereinafter, the steps of electrocardiogram measurement in which a parameter representing an arm to be worn is set in advance will be described. For convenience, the electrocardiogram measuring device may be expressed as a watch or a ring. As described above, in this embodiment, a parameter representing an arm to be worn is set in advance. This is done in step 461 of registering a watch or a ring to a smartphone. In this step, a user registers a watch or a ring to a smartphone, including the user's personal information.

As described above, the setting of an arm to be worn may be done by selecting a button displayed on a watch or a smartphone screen or by setting the display orientation of a watch. When an arm to be worn is selected, a parameter of the arm to be worn is stored in the memory of the watch, ring, or smartphone. This ends the setting of an arm to be worn.

Measurement of the ECG lead signals begins at step 463. In step 463, the user selects the number of ECG lead signals to be measured. This selection can be made by the user directly on a screen of a watch or a smartphone, or by determining the number of body parts touched by a current sensor. If only lead I is measured, the number of ECG lead signals to be measured is one. To get six ECG lead signals, the number of ECG lead signals to be measured is two. Depending on the number of ECG lead signals to be measured, the watch or ring starts taking an ECG measurement.

What the measured electrocardiogram lead signals are is determined by a parameter representing the arm wearing the watch or ring. Electrocardiogram lead signals measured in the four measurement steps shown in step 465 of FIG. 46 are all different. It is worth recalling that FIG. 46 shows four cases of electrocardiogram measurements that can be made in one watch or one ring. While measuring the ECG lead signals, the watch may present at least one ECG lead signal on a screen of the watch.

Electrocardiogram data measured in one of the four cases of step 465 may be transmitted to the smartphone. The ECG data transmitted by the watch or ring is numerical values obtained by sampling one or two ECG lead signals. The electrocardiogram data to be transmitted may include a parameter of the arm to be worn and the number of measured electrocardiogram leads. The watch or ring can communicate with a smartphone via Bluetooth low energy.

In step 467, the smartphone receives at least one measured ECG lead signal. The smartphone can determine what the received ECG is. The parameter of the worn arm may be stored in the memory of the smartphone or may be included in the received electrocardiogram data. Also, the received electrocardiogram data may include information about the number of measured electrocardiogram lead signals. Accordingly, in step 467, the smartphone determines the received electrocardiogram data and performs a subsequent operation according to the determined content. When lead I is measured, the sign of lead I can be determined. When two electrocardiogram leads are measured, the smartphone selects the calculation formulas to be used, and calculates, obtains, and displays each electrocardiogram lead signal.

The smartphone can store the measured data in a cloud server before ending the ECG measurement. Automatic diagnosis on the measured electrocardiogram data may be performed on a smartphone or a cloud server. The automatic diagnosis result may be transmitted to the medical staff as well as the user.

### Embodiments Measuring Lead I Only

Although all the embodiments described above are related to an electrocardiogram measuring device that obtains six electrocardiogram lead signals, it has been described that only lead I may be measured for convenience in the embodiment including the three electrocardiogram electrodes described above. In addition, just before, the electrocardiogram measuring device that a hand to be worn is set has been described. Hereinafter, conditions, devices, and methods necessary for embodiments in which only lead I is measured for convenience according to a user's selection will be described.

For convenience, it may be necessary to measure only the ECG lead signal between both hands. This includes cases in which some users can obtain sufficient information with only lead I, and it is inconvenient for a user to touch the electrocardiogram measuring device to the left leg or the user's clothes are unsuitable.

A number of embodiments using four ECG electrodes have been described above. In these embodiments, in order to measure only lead I, the electrode connected to the input of the electrode driver or the electrode connected to the output of the electrode driver should not come into contact with the left leg. This is because the electrode driver must form a feedback loop in the above-described embodiments which use the four ECG electrodes. If the left leg does not touch the electrocardiogram measuring device to measure only lead I, the feedback loop of the electrode driver is disconnected. Therefore, in order to measure only lead I, both the electrode connected to the input of the electrode driver and the electrode connected to the output of the electrode driver must be in contact with one hand or each of the two electrodes with each of both hands, respectively.

In all of the above embodiments, one electrode is in contact with the same human body part contacted by the electrode connected to the electrode driver output. Therefore, in order to measure only lead I, three electrodes must be in contact with the right hand (RA) and the left hand (LA). That is, two electrodes are in contact with one hand and one electrode is in contact with the other hand. That is, the electrode that is not used to measure lead I but is supposed to touch the left leg must be connected to the input of one amplifier.

For convenience, embodiments of measuring only lead I is described using Table 6. Among the seven symmetrical pairs shown in Table 6, there are three pairs wherein the electrode connected to the input of the electrode driver and the electrode connected to the output of the electrode driver do not contact the left leg. These are the embodiment pairs of A1 and A2, B1 and B3, and C1 and C4. Since these symmetrical pairs correspond to when the watch is worn by changing hands, it can be said that A2, B3, and C4 have actually different physical structures or configurations. That is, only these three are suitable for measuring lead I only. These three physical configurations have a common feature. The electrode connected to the input of the electrode driver and the electrode connected to the output of the electrode driver are in contact with one hand or each of the two electrodes with each of both hands, respectively.

In an electrocardiogram measuring device having a shape such as a ring, the ring may not include a display. The ring also may need to start an ECG measurement without a mechanical start switch. In particular, the ring needs to accommodate the user's intention to measure only lead I. In this case, only lead I can be measured if the contact of the left leg electrode to the left leg for a certain period of time is not detected after the contact of both hands is detected by the current detector.

As described above, the measurement of only one ECG lead signal, lead I, may be performed before or after determining which hand the ECG measuring device is worn on.

So far, for convenience, the necessity and method of measuring only Lead I have been described. In the case of a user whose electrical axis of the heart is in the direction of aVF, a warning message may be generated stating that only Lead I should not be measured. When a user measures Lead I, if the signal strength of Lead I is not strong enough, a warning message may be generated on a smartphone or smartwatch, saying “Since the signal strength of Lead I is not strong enough, you should not measure only Lead I”. Or, a warning message saying, “Touch the device with your left leg”, may be displayed.

In all the embodiments described so far or described in the following, the electrode driver can be implemented including digital signal processing. The ECG lead signal induced on one electrode is amplified by one amplifier, the output of the amplifier is AD-converted, digital signal processing is performed, and the output of the DA-converter is amplified to be used as an electrode driver output. This implementation can precisely control the output of the electrode driver.

One embodiment according to the above condition includes: An electrocardiogram measuring device in which an electrode connected to an input of an electrode driver and an electrode connected to an output of the electrode driver are in contact with one arm or both arms.

One embodiment includes: An electrocardiogram measuring device that measures only the electrocardiogram lead signal between both hands according to the user's choice, comprising: one electrode connected to the output of an electrode driver; and one electrode connected to the input of an amplifier that amplifies the electrocardiogram lead signal between both hands; are in contact with the same hand.

Alternatively, one embodiment includes the following: An electrocardiogram measurement device in which a current sensor detects contact between both hands to start an electrocardiogram measurement and measures only an electrocardiogram lead signal between both hands if contact of one electrode to the left leg is not detected.

### Tolerance Requirements

Now, it is known on which hand the wearable electrocardiogram measuring device such as a watch or a ring is worn, and accordingly, it is possible to determine what the measured two electrocardiogram lead signals are. Now, an embodiment in which four ECG voltages or ECG lead signals are calculated and obtained using the two measured ECG voltages will be described. A part of the following content is published in the same inventor's application (Publication No.: WO 2023/018318A1, Feb. 16, 2023, Application No.: PCT/KR2022/012211, Aug. 16, 2022).

First, the equations for the commonly known six electrocardiogram limb leads are summarized as follows. The following Equations 28 to 33 are described in ANSI/AAMI/IEC 60601-2-25:2011, Medical electrical equipment-part 2-25: Particular requirements for the basic safety and essential performance of electrocardiographs, which is an international medical device standard. The six equations are for six limb leads of the twelve leads. RA, LA, and LL are the voltages measured by an electrocardiograph in the right arm, left arm, left leg, or body parts close to these limbs, respectively.

$$I = L A - RA \qquad ( Equation 28 )$$

$$II = LL - RA \qquad ( Equation 29 )$$

$$III = LL - LA \qquad ( Equation 30 )$$

$$aVR = RA - ( L A + LL ) / 2 \qquad ( Equation 31 )$$

$$aVL = LA - ( R A + LL ) / 2 \qquad ( Equation 32 )$$

$$aVF = LL - ( RA + LA ) / 2 \qquad ( Equation 33 )$$

As described above, according to the present invention, after the two ECG lead signals measured are determined, four ECG lead signals are calculated and obtained. Formulas or equations for obtaining the four ECG lead signals have two cases as follows. It should be noted that the calculation formulas and results are different depending on the two measured ECG lead signals. Now, in this embodiment, an electrocardiogram measuring device and method for selecting and using formulas for additionally calculating and obtaining four electrocardiogram lead signals according to the two electrocardiogram lead signals measured by an electrocardiogram measuring device will be disclosed.

When the electrocardiogram measuring device according to the present embodiment measures lead I and lead II, four electrocardiogram leads are obtained using the following equations. For example, when the device in FIG. 24 is worn on the right hand, as shown on the right of FIG. 44, Lead I and Lead II are measured, so the following equations are used.

$$III = - I + II \qquad ( Equation 34 )$$

$$aVR = - ( I + II ) / 2 \qquad ( Equation 35 )$$

$$aVL = I - II / 2 \qquad ( Equation 36 )$$

$$aVF = - I / 2 + II \qquad ( Equation 37 )$$

The use of Equations 34 through 37 above in this embodiment is original. Thomson et al. disclosed Equations 35 through Equations 37. (U.S. Patent Application Publication, Pub. No.: US2015/0018660 A1, Pub. Date: Jan. 15, 2015, Appl. No.; Ser. No. 14/328,962, Claim 28.) However, Thomson et al. measure three voltages of RA, LA, and LL in order to use the above three equations. On the other hand, in this embodiment, only two ECG lead signals, that is, ECG voltages are measured. Therefore, this embodiment is more effective than Thomson et al. Also, Thomson et al. use Equation 30, III=LL-LA. That is, Thomson et al. do not use Equation 34. In the above, it was described that Thomson et al. use only Equations 35 to 37. In addition to Equations 34 to 37, the present invention discloses Equations 38 to 41 below. Therefore, this embodiment differs from Thomson et al.

When the electrocardiogram measuring device according to the present embodiment measures leads I and leads III, four electrocardiogram leads are obtained using the following equations. For example, the following equations are used when wearing the device in FIG. 24 on the left hand, as shown on the left of FIG. 44 and measuring-lead I and lead III.

$$II = I + III \qquad ( Equation 38 )$$

$$aVR = - I - III / 2 \qquad ( Equation 39 )$$

$$aVL = ( I - III ) / 2 \qquad ( Equation 40 )$$

$$aVF = I / 2 + III \qquad ( Equation 41 )$$

When the electrocardiogram measuring device according to the present embodiment measures leads II and leads III, four electrocardiogram leads may be obtained using equations different from the above equations. For convenience, this embodiment may be omitted.

In order to implement the above embodiments, there are several points to be noted. Expressing each term of Equation 38 as a function of time is as follows.

$$Lead II ( to + n T ) = Lead I ( to + n T ) + Lead III ( to + n T ) \qquad ( Equation 42 )$$

Equation 42 above means that in order to obtain another ECG lead from the two measured ECG leads, the two measured ECG leads must be sampled at the same time point. In Equation 42, T represents a sampling period and n represents a sampling number. Consider that the ECG measurement start command occurred at t=0. Then to represents the elapsed time until the first (n=0) sampling is performed (t=to). When the total number of samplings is N+1, NT represents the total time measured. In one embodiment, if the sampling rate is 300 sps (samples/second), T=3.333 ms. If measured for 30 seconds, N=30 s/3.333 ms=9,000.

Equation 42 indicates that the two ECG lead signals, Lead I and Lead III, are sampled at the same sampling rate. Therefore, in order to use Equations 34 through 41 in this embodiment, the ECG measuring device must sample two ECG lead signals at the same sampling rate. Of course, when the sampling rates are different, it is possible to use interpolation to convert the sampling rates to be the same. However, using the same sampling rate is much more effective.

After obtaining additional ECG leads by calculation, the six ECG leads can be displayed on a display of a smartphone. It can also be stored on a cloud server and a doctor can analyze six ECG leads in his or her office. The smartphone or the cloud server can automatically diagnose six ECG leads and provide the diagnosis results to the doctor. A doctor can be alerted when auto-diagnostic results indicate an emergency.

According to the above description, an embodiment includes: An electrocardiogram measuring device comprising at least three electrocardiogram electrodes, wherein the electrocardiogram measuring device selects equations for calculating and obtaining electrocardiogram lead signals according to a parameter representing a wearing arm.

From now on, in order to use Equations 34 to 37 or Equations 38 to 41, additional conditions required for an electrocardiogram measuring device including two amplifiers and two electrocardiogram lead signals measured using the device, are described.

A wearable device according to this embodiment is a medical device. An electrocardiogram measuring device implemented in the present embodiment must conform to a medical device certification standard. An international standard that may be applied at this embodiment is ANSI/AAMI/IEC 60601-2-47:2012, Medical electrical equipment-part 2-47: Particular requirements for the basic safety and essential performance of ambulatory electrocardiogram systems.

In order to implement this embodiment, the following conditions are required: The gains of the two amplifiers used in this embodiment must be the same.

When the two ECG lead signals measured using two amplifiers having unequal gains are applied to any one of Equations 34 to 37 above, an unsuitable result is obtained. Here, the gain includes the gain of the amplifier included in the electrocardiogram measuring device. The gain may refer to a final gain obtained after digital signal processing performed after AD conversion. In all the embodiments in this disclosure, the gain refers to a final gain that has gone through digital signal processing including a digital frequency filter. Therefore, the meaning of gain may include frequency response characteristics. The digital signal processing may not have to be performed in the same wearable device that has performed the AD conversion. The digital signal processing may be performed by smartphone application software connected to the electrocardiogram measuring device through wireless communication.

Before obtaining four ECG lead signals using the measured two ECG lead signals, digital signal processing may be performed on the measured two ECG lead signals. For example, digital signal processing must be performed to remove noises such as power line interference or motion artifacts described above. If not, noises also appear on the four calculated ECG leads. Once noise removal is performed on the measured two ECG lead signals, noise removal on calculated four ECG leads need not be performed. Noise can be removed from all six ECG leads, including calculated four ECG leads, by performing digital signal processing for noise removal on the measured two ECG leads. Digital signal processing to remove noise is not required for the four leads obtained using the above equations.

Besides the noise removal, the digital signal processing of the two measured ECG lead signals described above includes scaling conversion for displaying the ECG lead waveforms. For example, AD-converted digital values and the number of pixels on a display screen, corresponding to one mV, are determined. Thus, the digital signal processing includes normalization to display and store ECG lead signals in mV units. Such signal processing can be generally termed scaling or normalization. In this way, the gain also can be corrected in digital signal processing.

In this embodiment, a total of six ECG lead signals of the measured and the calculated ECG lead signals are displayed on a smartphone. When the electrocardiogram lead signals obtained in this embodiment are displayed on a smartphone, the same gain of the electrocardiogram measuring device means that when the same input signals are input to the two amplifiers, the two electrocardiogram waveforms displayed on the smartphone display are equal within a tolerance range. Accurate information about the ECG signal waveforms shown on the smartphone display can be obtained using detailed grids of the horizontal and vertical axes of the smartphone screen.

In this embodiment, when measuring electrocardiogram lead signals using a watch without a smartphone, one or two electrocardiogram lead signals may be displayed on the screen of the watch. In this case, the electrocardiogram lead signals displayed on the screen of the watch may include lead II, which is medically most important.

An embodiment includes: A watch including at least three electrocardiogram electrodes and showing lead II on the display of the watch.

When a smartphone and a watch are wirelessly connected, the ECG data obtained from the watch can be automatically transmitted to the smartphone. The smartphone may display six ECG lead signals using the transmitted ECG data.

In the above, the term expressed as the same or identical means that the magnitude of the difference between the two values is smaller than a tolerance or tolerable error. According to the above international standard, the gain accuracy must be within +/−10% of the maximum amplitude error. The compliance test for the above requirement in the international standard is performed with the following steps: “Apply a 5 Hz, 2 mV p-v sinusoidal signal to all ECG input channels.”

Accordingly, all the present embodiments have a feature of simultaneously amplifying two ECG lead signals using the identical two amplifiers. In order to use the above Equations 34 to 37 or Equations 38 to 41 for additionally obtaining ECG lead signals, two ECG lead signals measured simultaneously with the identical two amplifiers are required.

So far, we have understood the situation and introduced the concept, but we will now describe more strictly the contents described above. Although the term accuracy is used in the above standard, tolerance may be a more appropriate term than accuracy. Therefore, in the following, depending on the situation, the term tolerance or tolerance range may be used.

In addition, the term tolerance or tolerance range will be used by distinguishing two purposes. That is, the terms “tolerance required for a device” or “tolerance range required for a device” and “tolerance required for an amplifier” or “tolerance range required for an amplifier” will be used. The reason and meaning of using these terms will become more apparent in the embodiments below. The tolerance required for a device or the tolerance range required for a device means the tolerance required by the above international standard or a performance target required for an electrocardiogram measuring device to be manufactured by a company. On the other hand, the tolerance required for an amplifier or the tolerance range required for an amplifier refers to an allowable error of a measured electrocardiogram lead signal displayed on a smartphone screen after amplifying an electrocardiogram lead signal induced at an electrocardiogram electrode.

An example will be described using Table 7 in FIG. 47. Table 7 shows an example of errors in calculated ECG lead signals caused by errors in measured ECG lead signals. In the example of Table 7, when, as test signals, 0.60 mV to lead I and 1.00 mV to lead II are applied, measured results are lead I=0.54 mV and lead II=1.10 due to gain errors of the two used amplifiers. In this example, the accuracy of the two measurements is within the range permitted by the above international standards.

However, calculating aVF using Equation 37 using these measurement values gives 0.83 mV, which is 119% of the error-free value of 0.70m V. In this case, 19% of errors occurred. This error exceeds the tolerance of +/−10% of the international standard. If the measurement tolerance of lead I and lead II is 5%, we can obtain aVF=0.765 mV according to Equation 37. Then, the obtained error is 9%, and the requirement of the international standard can be satisfied.

Therefore, when implementing an embodiment, the accuracy of the gains of the two amplifiers is required to be superior to the international standard. For example, the tolerance of the gains of the two amplifiers must be within +/−5%. In this case, the maximum measurement tolerance or the tolerance required for an amplifier is 5%.

The conclusion drawn from the example of Table 7 is that the tolerance for the calculated ECG lead signals must satisfy the “tolerance required for a device” of the ECG measuring device, and to do so, the tolerance for the measured ECG lead signals must be less than the “tolerance required for a device”. What the tolerance of the measured ECG lead signals must satisfy is the “tolerance required for an amplifier”. Therefore, the “required tolerance of an amplifier” is less than the “required tolerance of the device” and is about half.

Table 7 shows a situation that may occur when actually measuring ECG lead signals. However, it may be difficult to draw a general conclusion because the magnitudes of ECG lead signals vary according to users in actual measurement environments. Therefore, the error analysis shown in Table 7 can be expanded by using two identical inputs to the two electrocardiogram electrodes. Even in this approach, the same conclusions as those obtained in Table 7 can be drawn. That is, the error of the calculated ECG lead signals is always greater than the error of the measured ECG lead signals.

Accordingly, the present embodiment includes: an electrocardiogram measuring device including at least three electrocardiogram electrodes; and two amplifiers having an identical gain within a tolerance range required for an amplifier.

When an identical input signal is applied to two amplifiers having the same gain, the waveforms of the two ECG lead signals displayed on a smartphone screen should be identical. Therefore, this embodiment includes: An ECG measurement device comprising: at least three electrocardiogram electrodes; and two amplifiers connected to two electrocardiogram electrodes among the at least three electrocardiogram electrodes, wherein, when an identical input signal is applied to the two amplifiers, the magnitudes of the two measured electrocardiogram lead signals displayed on a smartphone screen are identical with a tolerance required for an amplifier.

In order for the two amplifiers to have the same characteristics, the two amplifiers need to have the same structure or components. Therefore, this embodiment includes: An electrocardiogram measuring device including two amplifiers having the same components.

The amplifiers included in this embodiment are basically manufactured using operational amplifiers. In amplifiers using operational amplifiers, errors in amplifier characteristics, such as gain and frequency response, are mainly caused by errors in component values of resistors and capacitors. However, the error of these amplifiers can be controlled by designing and verifying by computer simulation considering tolerances of component values. To this end, resistors and capacitors may be high-precision, low-tolerance elements. The tolerance for resistors and capacitors can be +/−1%. Operational amplifiers may be low-noise amplifiers. The input impedance of the two amplifiers must be greater than 10 MOhm. The same two amplifiers required for this embodiment can be manufactured by applying these considerations.

In order to implement this embodiment, the following conditions are required. The gain accuracy of the two amplifiers used must be superior to the gain accuracy required by the international standard. For example, the maximum amplitude error of the two amplifiers must be within +/−5%. Otherwise, the accuracy of the ECG lead signals calculated using Equations 34 to 37 may have a maximum amplitude error larger than +/−10%. This can be understood from the example in Table 7.

From the above example, it can be seen that the allowable error of the measured ECG lead signals must be smaller than the allowable error required for the device in order to obtain the calculated ECG lead signals having the allowable error required for the device. In the following, the electrocardiogram lead signals obtained from the electrocardiogram measuring device according to the present embodiment will be separately referred to as measured electrocardiogram lead signals and calculated electrocardiogram lead signals.

It should be noted that the above situation occurs even if three ECG lead signals are measured and the three ECG lead signals are obtained by calculation. In addition, the above situation occurs regardless of whether an electrocardiogram lead signal to be measured is a limb lead or an augmented limb lead. Therefore, this embodiment includes an embodiment of measuring three electrocardiogram lead signals and an embodiment of measuring an augmented limb lead.

If the two ECG lead signals measured in the foregoing embodiment were measured completely accurately, the additional four ECG lead signals obtained by calculation would also be completely accurate. However, since an error due to a measurement device always exists, an error occurs in the measured value, and the additional four ECG lead signals obtained by calculation also have an error. The accuracy of the electrocardiogram lead signals measured in the above-described embodiment is required to be superior to the required standard. That is, in order to obtain calculated electrocardiogram lead signals having accuracy required for the device, the accuracy of the measured electrocardiogram lead signals must be higher.

The accuracy of a calculated ECG lead signal is always lower than the accuracy of the measured ECG lead signals. The accuracy of a measuring device is an important specification of the measuring device. Therefore, when indicating the specifications of an electrocardiogram measurement device, the accuracy of the calculated electrocardiogram lead signals must be indicated. Alternatively, the accuracy of the measured electrocardiogram lead signals and the accuracy of the calculated electrocardiogram lead signals should be recorded together.

Likewise, although the measured two ECG lead signals are measured actually, it is not an accurate expression to express the accuracy of the measured two ECG lead signals as the accuracy of the ECG measurement. In order not to confuse the user, the final and accurate result should be informed. Measured ECG lead signals are not the final results. The final result is the computed electrocardiogram lead signals. Therefore, the accuracy of the calculated electrocardiogram lead signals should be informed and expressed as the accuracy of the electrocardiogram measuring device. The accuracy of the electrocardiogram measuring device should be expressed as the accuracy of the calculated electrocardiogram lead signals, which shows larger and worst-case errors. Two accuracies may be informed by distinguishing the measured ECG leads from the calculated ECG leads.

From now on, the accuracy required for the calculated ECG leads may be informed as the allowable error of the ECG measuring device. Therefore, the tolerance required for the calculated ECG leads is the same as the tolerance of the ECG measurement device. Thus, the tolerance required for the measured electrocardiogram leads should be smaller than the tolerance of the electrocardiogram measuring device. The tolerance required for the measured ECG leads may be determined as half of the tolerance of the ECG measuring device.

The accuracy of the calculated electrocardiogram leads should be determined as the accuracy required for the electrocardiogram measuring device. When tested by a medical device certification office or presenting test results independently, the accuracy of the measured ECG lead must not be presented. The accuracy of the calculated ECG leads should be presented.

The accuracy of the calculated ECG leads, as well as the accuracy of the measured ECG leads, can be measured by looking at the smartphone screen. Alternatively, automated testing software may be used.

In order to increase the accuracy of the measured electrocardiogram leads, it may be necessary to consider the selection of parts to be used from the design stage of the electrocardiogram measuring device. Even when testing the accuracy of the electrocardiogram measuring device after production of the electrocardiogram measuring device, it should be considered that the accuracy of the measured electrocardiogram leads should be higher than the accuracy of the calculated electrocardiogram leads.

According to the above, an embodiment includes: An electrocardiogram measuring device for obtaining a calculated electrocardiogram lead having a predetermined tolerance and measured electrocardiogram leads having a tolerance smaller than the predetermined tolerance.

In addition, an embodiment includes: An electrocardiogram measurement device for obtaining calculated electrocardiogram leads that satisfy a predetermined tolerance, and measured electrocardiogram leads that satisfy a tolerance that is approximately half of the predetermined tolerance.

The error generated by an AD converter or a smartphone microprocessor performing the calculation is considerably smaller than the allowable error of the electrocardiogram measuring device. Therefore, an embodiment includes: An electrocardiogram measurement device including two equivalent amplifiers compliant to a tolerance smaller than the tolerance of the electrocardiogram measurement device.

In addition, an embodiment includes: An electrocardiogram measuring device including two amplifiers having a gain of accuracy higher than the accuracy of the calculated electrocardiogram leads.

The present embodiment includes: An electrocardiogram measuring device in which measured electrocardiogram leads and calculated electrocardiogram leads are displayed on a smartphone screen, and a tolerance of the measured electrocardiogram leads is smaller than a tolerance of the calculated electrocardiogram leads.

An embodiment includes: An electrocardiogram measurement device in which calculated electrocardiogram leads displayed on a smartphone display satisfy a tolerance determined according to device requirements.

An embodiment includes: An electrocardiogram measuring device, including at least three electrocardiogram electrodes contacting three body parts of the right arm, left arm, and left leg, wherein calculated electrocardiogram lead signals, calculated by using measured electrocardiogram lead signals, have a tolerance compliant with the requirements of the device.

To implement an embodiment, the following conditions are required: The frequency response characteristics of the two amplifiers must be the same within the “tolerance range required for the amplifier”. If two ECG lead signals measured by two amplifiers having unequal frequency response characteristics are applied to any one of Equations 34 to 37 above, an unsuitable result is obtained. According to the above international standard, the frequency response requirements for testing with sine waves are as follows: The amplitude response within the frequency range of 0.67 Hz to 40 Hz shall be between 140% and 70% of the amplitude response at 5 Hz. The test is performed at 0.67 Hz, 1 Hz, 2 Hz, 5 Hz, 10 Hz, 20 Hz, and 40 Hz.

According to the above, an embodiment includes: An electrocardiogram measuring device including two amplifiers of which an amplitude response of a frequency response is identical within a “tolerance range required for an amplifier”.

When an input signal is applied to two amplifiers with the same frequency response, the two measured ECG voltage waveforms displayed on a smartphone screen must be identical within a tolerance range. This means that the two measured ECG voltage waveforms displayed on a smartphone screen must satisfy more stringent requirements than the frequency response required by the above international standard.

Therefore, an embodiment includes: An electrocardiogram measurement device wherein, when an input signal is applied to two amplifiers, the two frequency responses of the two measured electrocardiogram voltages displayed on a smartphone screen, are the same within a tolerance range required for an amplifier.

To implement an embodiment, the following conditions are required: the frequency response characteristics of two amplifiers used in the embodiment must have less tolerance than the requirements of the above international standard. The reason is the same that an amplifier having a gain characteristic of better accuracy is required, as described above. This is because the allowable error of the calculated ECG lead signals is greater than the allowable error of the measured ECG lead signals. For example, the amplitude response of two amplifiers in the frequency range of 0.67 Hz to 40 Hz may be within 120% and 85% of the amplitude response at 5 Hz.

One embodiment includes: An electrocardiogram measurement device including two amplifiers having a frequency response tolerance smaller than a tolerance of the electrocardiogram measurement device.

Incorporating the embodiments described so far, the present embodiment includes: An electrocardiogram measuring device including two amplifiers having the same characteristics within a tolerance range smaller than a tolerance range required by an international standard.

In addition, an embodiment includes: An electrocardiogram measurement device including a smartphone that performs an operation of calculating additional electrocardiogram lead signals using measured electrocardiogram lead signals and an operation of displaying six electrocardiogram lead signals on its screen.

Arrhythmias may be intermittent and asymptomatic. Accordingly, a photoplethysmograph (PPG) may be mounted on a watch or a ring and the pulse or cardiac activity may be continuously monitored using the PPG. The PPG has the advantage of being able to measure photoplethysmograms simply by wearing it in one hand. If PPG, which has been monitoring cardiac activity, detects an abnormality in cardiac activity, that is, it detects the occurrence of arrhythmias, the PPG can generate an alarm. The alarm may be in the form of sound, vibration, or light. After detecting the alarm a user can measure an electrocardiogram. In particular, in this embodiment, six ECG lead signals can be obtained.

Upon sensing the alarm, the user puts the opposite hand wearing the watch or the ring in contact with the corresponding electrode of the watch or the ring. Then, a current sensor installed in the electrocardiogram measuring device detects the contact of the opposite hand. The current sensor makes a microprocessor prepare an electrocardiogram measurement for lead I and attempt a Bluetooth Low Energy connection with a smartphone. Also, when the user makes the corresponding electrode to touch the left leg, a current from the current sensor flows between the left leg and a hand. Then, the current sensor detects the contact of the left leg and generates a corresponding output. The microcontroller performs an electrocardiogram measurement for lead II or lead III after preparing for the electrocardiogram measurement.

As described above, embodiments of a device and method for obtaining four ECG leads by measuring at least two ECG leads and calculating the measured at least two ECG leads have been disclosed. To this end, in the above embodiments, at least three electrocardiogram electrodes contacting three human body parts were used.

As described above, it was shown that the present invention can be implemented in a variety of ways. According to the present invention, an embodiment may be configured differently from the embodiments shown so far. One embodiment can be selected that is suitable for one particular design of a wearable device. An electrocardiogram measuring device according to the present invention is convenient to carry, can be easily used regardless of time and place, and can be used as a portable electrocardiogram measuring device capable of obtaining electrocardiogram information of multiple ECG signals.

### Embodiments Including a Differential Amplifier

As described above, the embodiments previously explained may include a single differential amplifier. Hereinafter, embodiments including one differential amplifier will be described in detail. When three electrodes are brought into contact with the right hand, left hand, and left leg, three differential voltages exist, since there are three possible pairs among the three electrodes. Embodiments may be conceived in which three differential amplifiers are provided to measure the three differential voltages, respectively. However, it is not necessary to use three differential amplifiers, because, as previously explained, the voltage corresponding to the third differential amplifier can be obtained by calculation when two differential amplifiers are used.

In addition, embodiments employing three differential amplifiers are included in embodiments employing two amplifiers. Even if three amplifiers are used, no more accurate or richer information is obtained than when two amplifiers are used. The electrocardiogram measured with the additional amplifier is merely redundant. Nonetheless, the embodiments employing three amplifiers are included in the embodiments employing two amplifiers, though they provide no additional benefit as previously described. The present invention may be expressed as follows:

An electrocardiograph comprising at least three electrodes, measuring three electrocardiogram signals, and further obtaining three electrocardiogram signals by calculation.

A hand and an arm are electrically and electrocardiographically equivalent. And a left leg, left foot, left ankle, or left lower abdomen are also electrically or electrocardiographically equivalent. Therefore, these terms may be used interchangeably for convenience depending on the context.

Embodiment Group F shown in FIGS. 41 through 43 has been described previously. Embodiment Group F will now be described in detail. First, Embodiment F3 shown in FIG. 41, which includes a differential amplifier, will be described in detail.

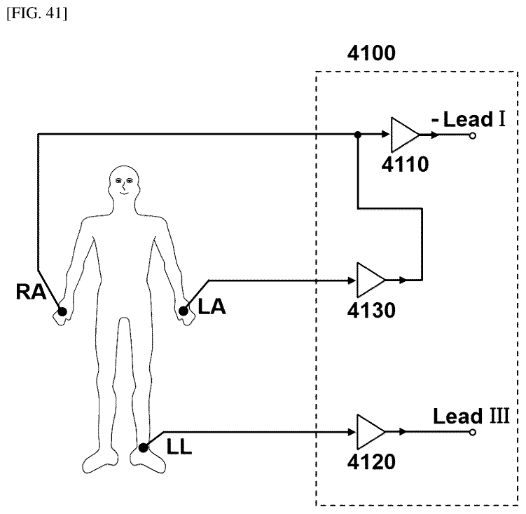

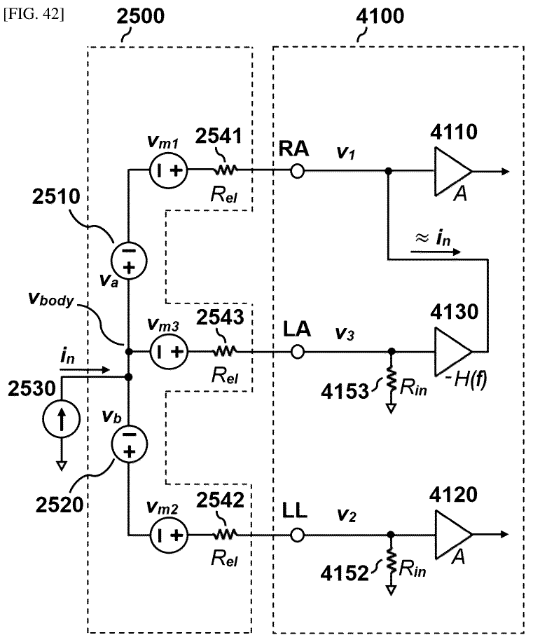

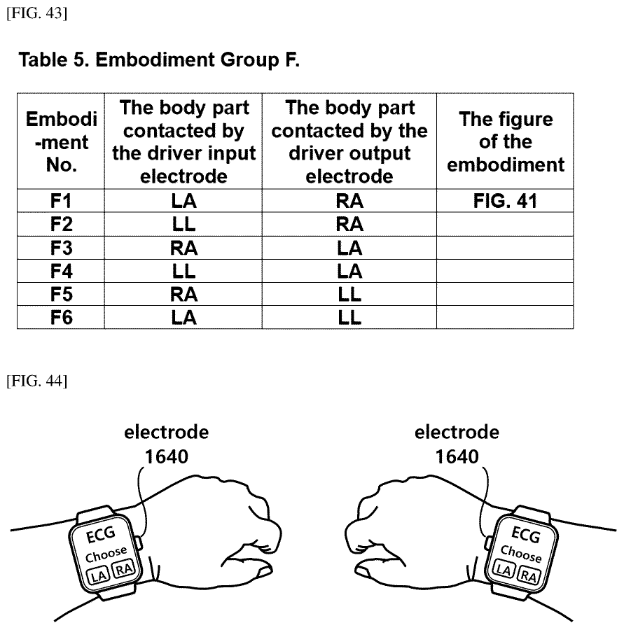

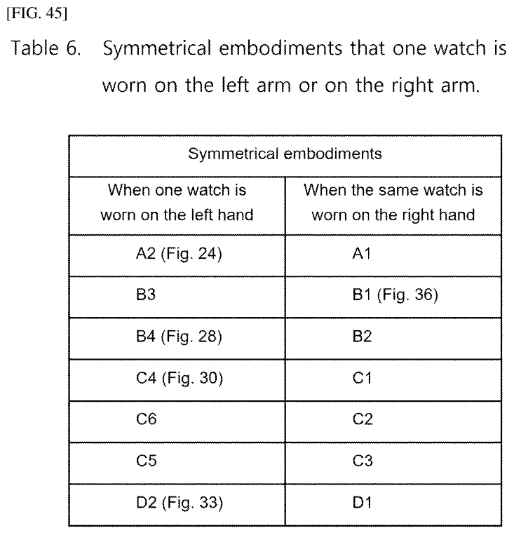

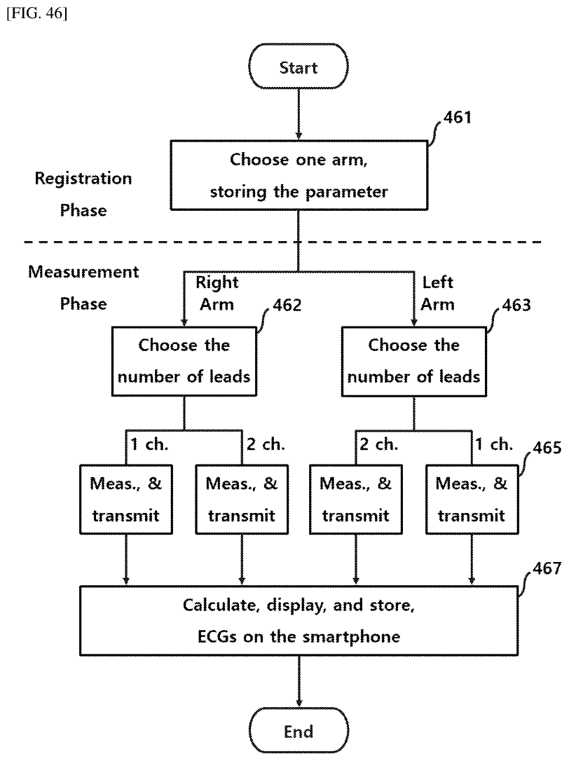

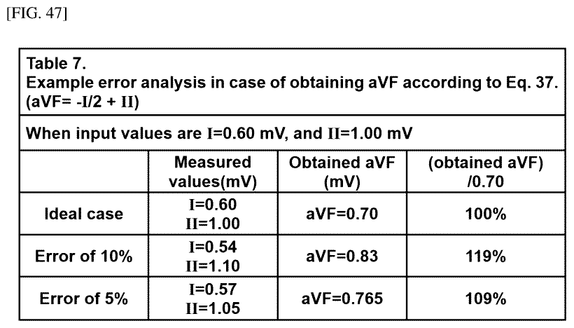

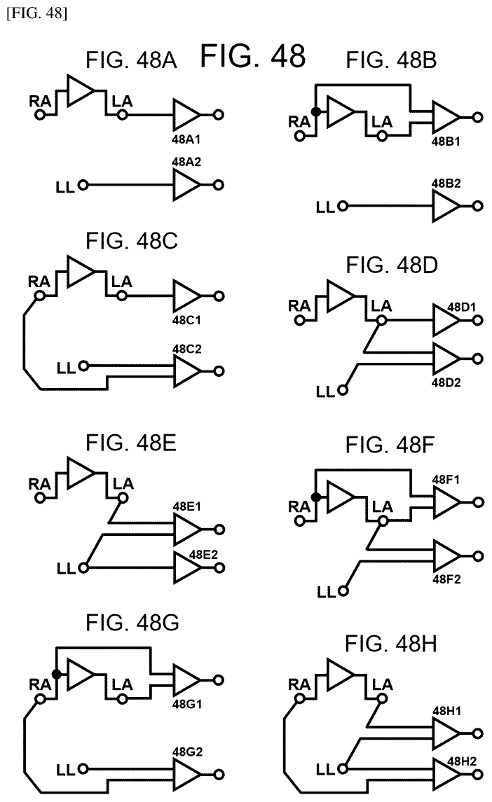

FIG. 48 illustrates subset embodiments of Embodiment F3, including two amplifiers, wherein the embodiment includes three electrodes, one electrode driver is one amplifier, the input of the electrode driver is connected to the right hand electrode, and the output is connected to the left hand electrode. The embodiments shown in FIG. 48 are variations of Embodiment F3. Although the embodiments shown in FIG. 48 are various, they still belong to Embodiment F3. Therefore, the embodiments shown in FIG. 48 are referred to as subset embodiments of Embodiment F3. As shown in FIG. 43, Embodiment F3 is characterized in that the input of the electrode driver is connected to RA, and the output of the electrode driver is connected to LA. As will be described below, Embodiment F3 provides a lead II in which power-line interference is almost completely eliminated.

There are eight subset embodiments of Embodiment F3. FIG. 48 illustrates eight subset embodiments belonging to Embodiment F3. Embodiments according to the present invention include two amplifiers. Therefore, all embodiments according to the present invention directly measure two ECG lead signals. Accordingly, the eight embodiments shown in FIG. 48 also have these characteristics.

The embodiment of FIG. 48A includes two single-ended input amplifiers. Each of the two amplifiers of Embodiment 48A amplifies Lead I and Lead II, respectively. Accordingly, Embodiment 48A directly measures Lead I and Lead II. Importantly, all eight embodiments of FIG. 48 obtain Lead II in which power-line interference is eliminated. This will be described in further detail below.

The four embodiments shown in FIGS. 48B, 48C, 48D, and 48E each include one differential amplifier and one single-ended input amplifier. In Embodiments 48B and 48C, the two amplifiers respectively amplify Lead I and Lead II. Therefore, Embodiments 48B and 48C directly measure Lead I and Lead II. Embodiment 48D directly measures Lead I and Lead III. Embodiment 48E directly measures Lead II and Lead III.

Among the embodiments shown in FIG. 48, Embodiments 48F, 48G, and 48H include two differential amplifiers. These also directly measure two lead signals. Even in these embodiments, using two differential amplifiers provides no advantage over using single-ended input amplifiers.

With each of the eight subset embodiments of Embodiment F3 described above, Lead II with further reduced power-line interference is obtained, as expressed by Equation 54. Accordingly, although the number and connection of the differential amplifiers differ among the eight subset embodiments of Embodiment F3, they are considered equivalent to each other. Similarly, in the eight subset embodiments of Embodiment F4, Lead II with further reduced power-line interference is obtained. A detailed analysis thereof will be omitted.

In contrast, in the subset embodiments of embodiments F5 and F6, where the output of the electrode driver is connected to the left leg electrode LL, Lead II with further reduced power-line interference cannot be obtained. This is because the output of the electrode driver connected to the left leg electrode includes power-line interference.

As described above, the embodiment of FIG. 48A can measure only Lead I. Among the embodiments shown in FIG. 48, Embodiments 48A, 48B, 48C, 48D, 48F, and 48G are capable of measuring only Lead I, whereas Embodiments 48E and 48H are not capable of measuring only Lead I. Of course, in these embodiments, Lead I can still be obtained by calculation using the two measured lead signals.

When measuring only Lead I, it is unnecessary to select a computational formula, and no additional lead signal may be derived. Deep-learning training or analysis is also performed using only Lead I. In this case, conventional data, such as Lead II, cannot be used for training data.

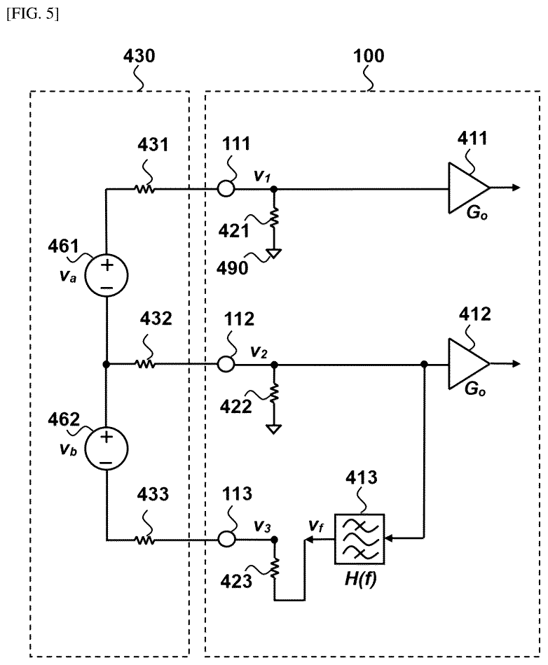

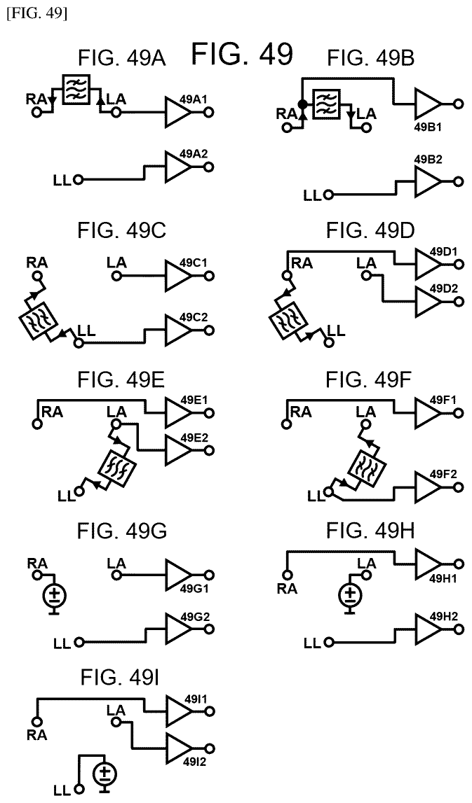

FIG. 49 illustrates embodiments of Embodiment Group H, which include three electrodes, an electrode driver, and two single-ended input amplifiers, wherein the electrode driver is a band-pass filter or a constant voltage source, and an output of the electrode driver is connected to an electrode. The embodiments described in FIGS. 4 and 5 are classified as Embodiment Group H. Specifically, the embodiment described in FIGS. 4 and 5 corresponds to Embodiment 49E in FIG. 49. Group H uses either a band-pass filter or a constant voltage source as the electrode driver. Group H includes various embodiments, as shown in FIG. 49, depending on which electrodes are connected to the input and output of the band-pass filter, or to which electrode the output of the constant voltage source is connected. Embodiments employing at least one differential amplifier or at least one voltage divider are described later as different groups.

As described previously, when the impedance observed through the electrodes toward the electrocardiogram measurement apparatus is high, power-line interference increases. Therefore, in Group H, an element having a low output impedance that supplies voltage to provide a reference voltage—that is, an AC common signal—is used, specifically a band-pass filter or a constant voltage source. In the present invention, one electrode driver may be implemented as a band-pass filter, a constant voltage source (or constant voltage generator), or an amplifier.

In all embodiments including a constant voltage source, the constant voltage source provides an AC circuit common. As emphasized earlier, it allows the power-line interference current to flow through a low impedance path. Embodiment 49E corresponds to the embodiments shown in FIGS. 4 and 5, in which the principle of power-line interference elimination has been described in detail.

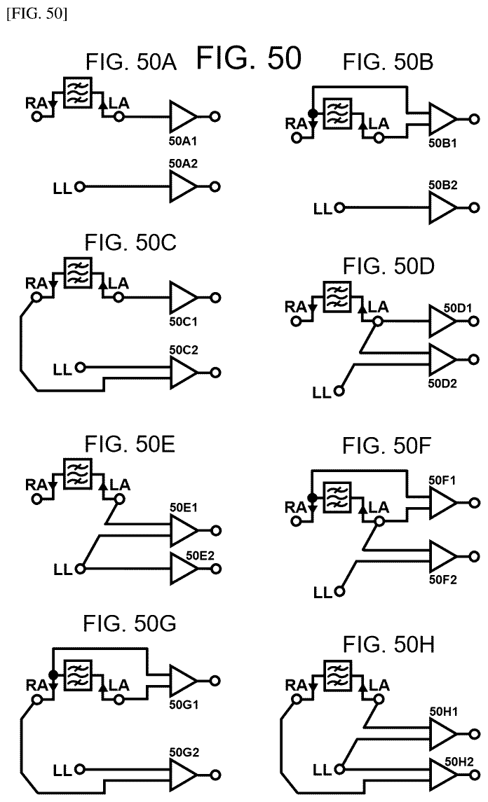

FIG. 50 illustrates subset embodiments of Embodiment 49A, which include three electrodes, an electrode driver, and two amplifiers, wherein the electrode driver is a band-pass filter, an input of the electrode driver is connected to a left-hand electrode, and an output thereof is connected to a right-hand electrode. The subsets of Embodiment 49A shown in FIG. 49 include eight subset embodiments illustrated in FIG. 50. All of these eight subset embodiments share the common configuration in which the input of the band-pass filter is connected to the left-hand electrode and the output of the band-pass filter is connected to the right-hand electrode. These embodiments include at least one single-ended input amplifier or at least one differential amplifier.

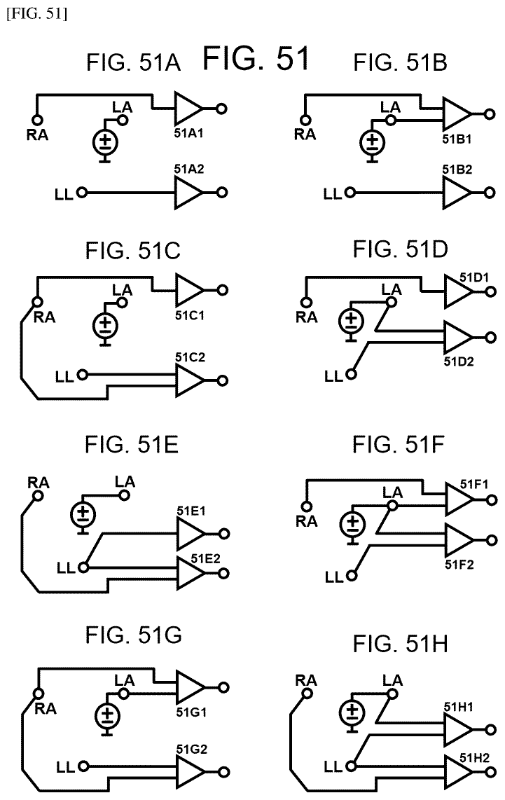

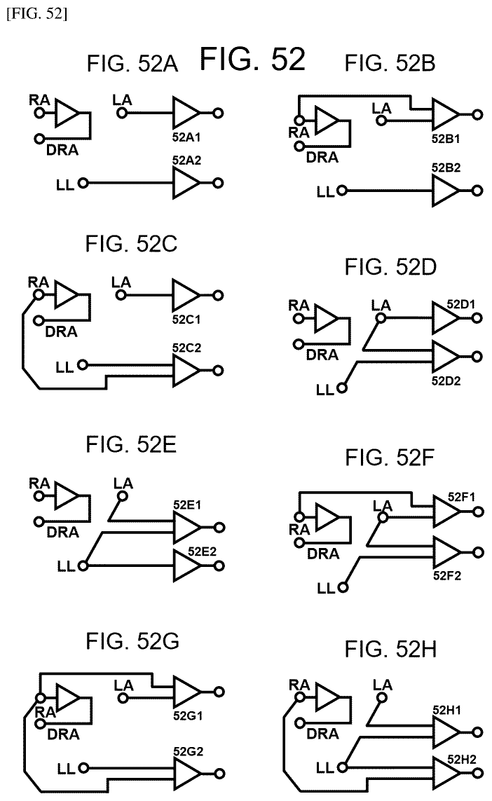

FIG. 51 illustrates subset embodiments of Embodiment 49H, which include three electrodes, an electrode driver, and two amplifiers, wherein the electrode driver is a constant voltage source, and an output of the constant voltage source is connected to a left-hand electrode. The number of subset embodiments shown in FIG. 52 is also eight. These eight embodiments have in common that the output of the constant voltage source is connected to the left-hand electrode.

The embodiment of FIG. 51A is identical to Embodiment 49H. A detailed analysis of power-line interference in this embodiment is described later. According to that analysis, Embodiment 49H—i.e., the embodiment of FIG. 51A—obtains Lead II, in which most of the power-line interference is eliminated. This analytical result applies to all eight embodiments of FIG. 51. In other words, although the connections of the included amplifiers differ, all eight embodiments of FIG. 51 have the same power-line interference reduction characteristics and thus can be regarded as equivalent in this aspect.

FIG. 52 illustrates subset embodiments of Embodiment A1 of FIG. 27, which include four electrodes, an electrode driver, and two amplifiers, wherein the electrode driver is an amplifier, an input of the electrode driver is connected to a right-hand electrode, and an output thereof is connected to another right-hand electrode disposed adjacent to the right-hand electrode.

The number of subset embodiments shown in FIG. 52 is also eight. The input of the electrode driver connected to the right-hand electrode provides a virtual ground. The amplifier used as the electrode driver has a high input impedance and a low output impedance. Accordingly, the voltage of the human body (v_body) induced by the power-line interference current becomes small. Thus, even though the included amplifiers differ, the embodiments shown in FIG. 52 provide nearly equivalent and excellent power-line interference elimination performance.

In the embodiments of Groups A through D, employing four electrodes, one input terminal of one differential amplifier may be connected to the input of the electrode driver. FIG. 52 shows such embodiments as well as other variations.

To measure two electrocardiogram voltages in which power-line interference is more effectively removed, four electrodes should be used according to the embodiments of Groups A through D. However, though power-line interference is severe in hospital-use electrocardiographs powered by the AC mains, in wearable electrocardiographs powdered by a battery, power-line interference is relatively weak. Therefore, in wearable electrocardiographs, as in the embodiments of Groups F through J of the present invention, three electrodes are sufficient to obtain six limb leads having a satisfactory signal-to-noise ratio (SNR). In any embodiment, additional reduction of power-line interference and motion artifact can be achieved through digital signal processing.

As described earlier, in embodiments including three electrodes (as in FIGS. 4 through 10), when two or more differential amplifiers are used without connecting the output of one electrode driver to one electrode, power-line interference becomes too large to measure electrocardiogram signals. However, when the output of one electrode driver is connected to one electrode, two differential amplifiers may be used. Of course, in such embodiments, the use of a differential amplifier may not necessarily be advantageous for reducing power-line interference. Further details will be described later in connection with the analysis of power-line interference elimination.

### Embodiments Including a Voltage Divider

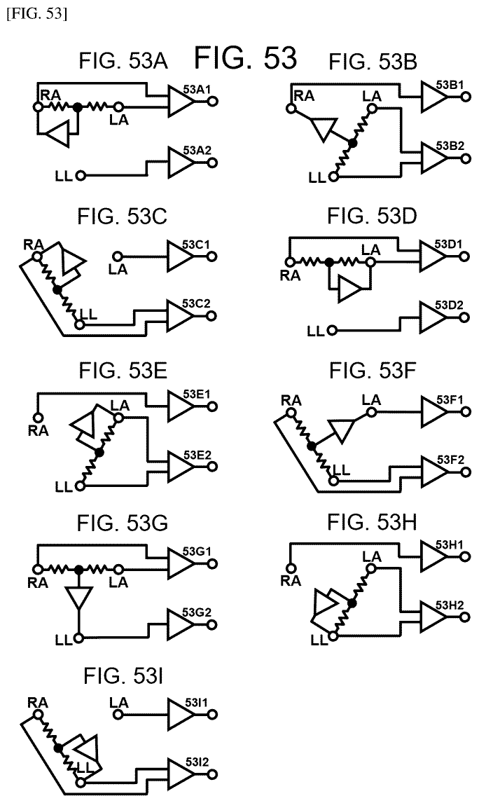

Embodiments including one voltage divider will now be described. FIG. 53 illustrates embodiments including three electrodes, wherein one electrode driver is one amplifier, the input of the electrode driver is connected to the center point of one voltage divider, the output of the electrode driver is connected to one electrode, and the embodiments further include one differential amplifier and one single-ended input amplifier.

Previously, embodiments of a wearable electrocardiograph employing three electrodes—i.e., Embodiment Group F shown in FIGS. 41 through 43—were disclosed. Hereinafter, further embodiments based on those of FIGS. 41 through 43 are disclosed in FIG. 53, and these embodiments are referred to as Group G. The embodiments of Group G differ from those of Group F in that they employ one voltage divider. The output of the electrode driver may be connected to one end of the voltage divider or to an electrode opposite the voltage divider. Group G may also include embodiments that incorporate two differential amplifiers.

Embodiments employing three electrodes and a voltage divider are substantially similar to the embodiments employing four electrodes and a voltage divider shown in FIGS. 30 through 34. In the embodiments employing four electrodes, two electrodes are brought into contact with one body part. As described previously, when two electrodes contact one body part, one electrode is connected to the output of the electrode driver, and the other electrode is connected to the input of one amplifier or to the voltage divider. If these two electrodes are combined into one, the embodiment employing four electrodes is transformed into an embodiment employing three electrodes.

In Group C, a voltage divider is composed of two resistors. Therefore, embodiments employing three electrodes and a voltage divider composed of two resistors can be regarded as configurations in which an electrode connected to the output of the electrode driver in Group C is combined with another electrode contacting the same body part. These embodiments are defined as Group G. In Group D, Wilson Central Terminal is composed of three resistors. Wilson Central Terminal is essentially a voltage divider consisting of three resistors. Accordingly, embodiments similar to Group D of FIG. 34 and employing three electrodes are also conveniently included in Group G. Among the twelve embodiments belonging to Group G, nine are shown in FIG. 53.

In Embodiments 53B, 53F, and 53G belonging to Group G, the input of one amplifier is connected to an electrode that is not connected to the voltage divider. The outputs of these amplifiers correspond respectively to aVR, aVL, and aVF. These embodiments have the advantage of directly measuring one of the augmented limb leads (aVR, aVL, aVF). This is a common characteristic also possessed by the embodiments of Group C employing four electrodes. As will be described later, the embodiments of FIG. 53 may also include a differential amplifier connected across both ends of the voltage divider.

From these embodiments, six limb-lead electrocardiogram (ECG) signals can be obtained. Taking Embodiment 53G as an example: Lead I can be measured by one differential amplifier connected across both ends of the voltage divider. Lead aVF can be measured at the output of one single-ended input amplifier connected to the left-leg electrode (LL). Then, the remaining four lead signals can be calculated using the following equations.

From Equation (41), the following relationship is obtained:

$$III = - I / 2 + aVF \qquad ( Equation 43 )$$

Thus, the following equations are derived:

$$II = I + III = I / 2 + aVF \qquad ( Equation 44 )$$

$$aVR = - 3 I / 4 - aVF / 2 \qquad ( Equation 45 )$$

$$aVL = 3 I / 4 - aVF / 2 \qquad ( Equation 46 )$$

In the above equations, Leads II and III are obtained by calculation. It is believed that, prior to the present invention, there was no conventional technique that obtained two or more limb-lead signals by calculation using at least one measured augmented-lead signal. In other words, there appears to have been no prior art corresponding to the embodiments of FIG. 53G.

Meanwhile, in six Embodiments 53A, 53C, 53D, 53E, 53H, and 53I, the output of one electrode driver is connected to one end of the voltage divider.

Among the remaining three embodiments of Group G, not shown in FIG. 53, a voltage divider composed of three resistors is used. In these embodiments, one single-ended input amplifier may be connected to each of the two electrodes other than the electrode to which the output of the electrode driver is connected. These embodiments are similar to those of Group D shown in FIG. 33. At least one differential amplifier may also be used in these embodiments.

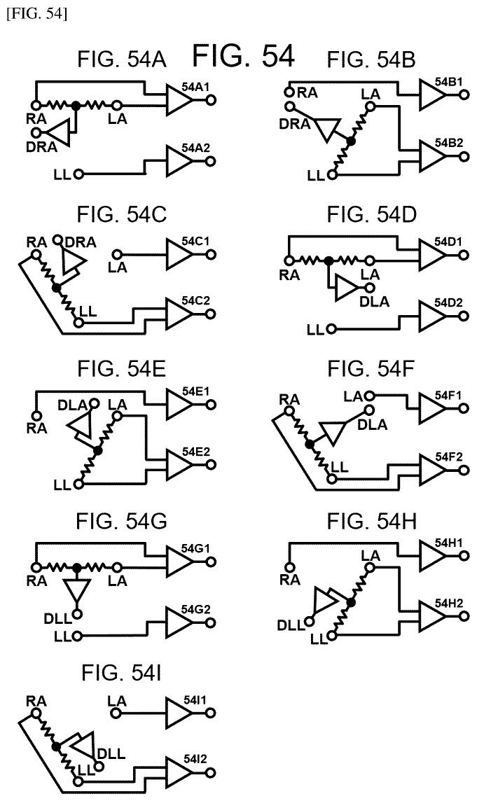

FIG. 54 illustrates subset embodiments of Embodiment Group C, which include four electrodes, an electrode driver, a differential amplifier, one single-ended input amplifier, and one differential amplifier, wherein the electrode driver is an amplifier, an input of the electrode driver is connected to a center point of a voltage divider, and an output thereof is connected to a fourth electrode. In the embodiment of FIG. 54, if the RA electrode and the DRA electrode are combined, the configuration becomes equivalent to the embodiment of FIG. 53.

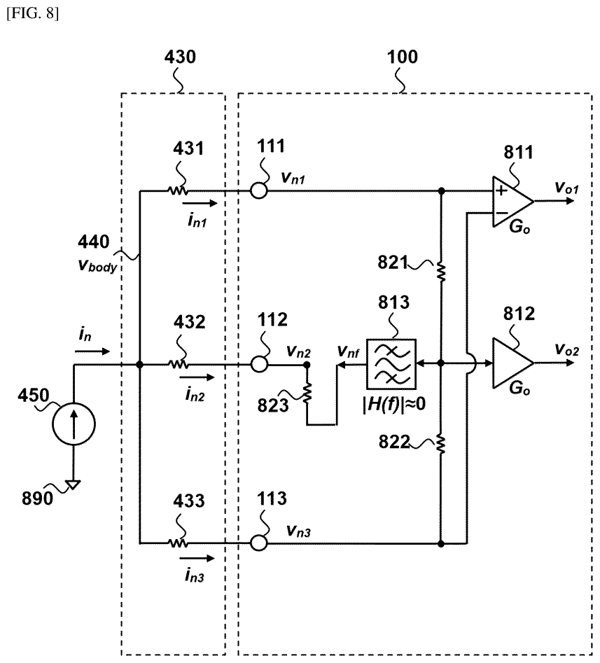

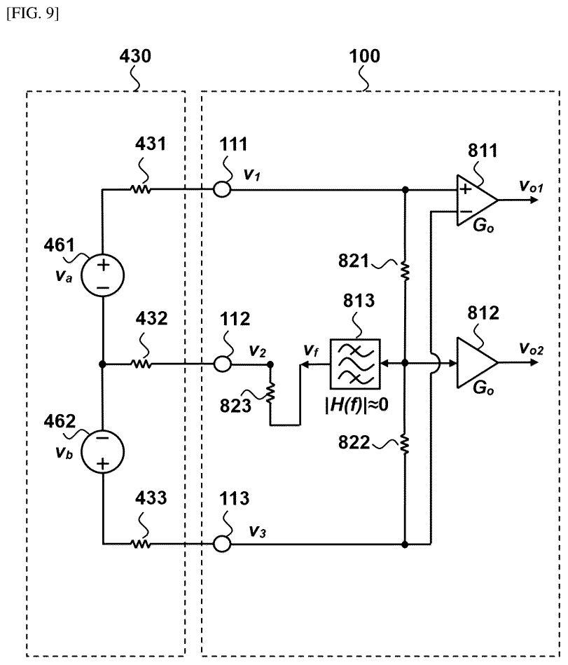

FIG. 55 illustrates embodiments of Embodiment Group I, which include three electrodes, an electrode driver, a differential amplifier, and a single-ended input amplifier, wherein the electrode driver is a band-pass filter, an input of the band-pass filter is connected to a center point of a voltage divider, and an output thereof is connected to an electrode. The embodiment of FIG. 55F corresponds to the embodiments shown in FIGS. 8 and 9.

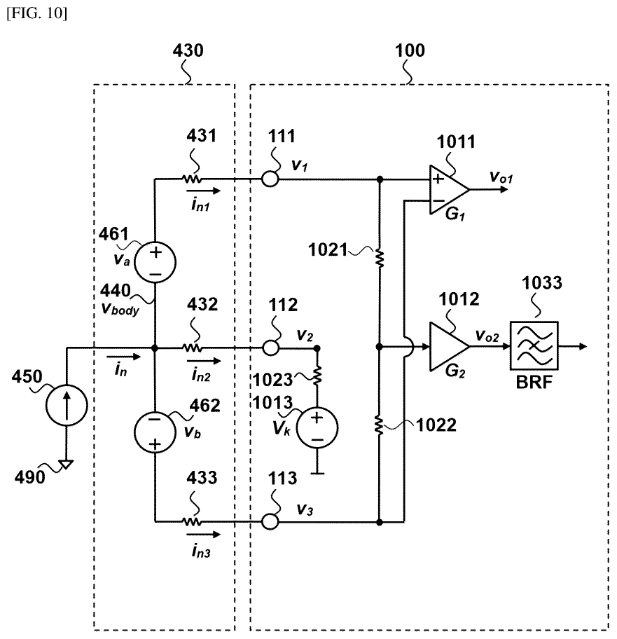

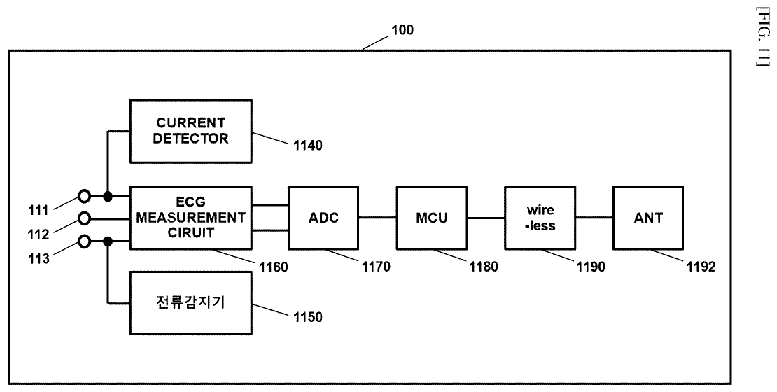

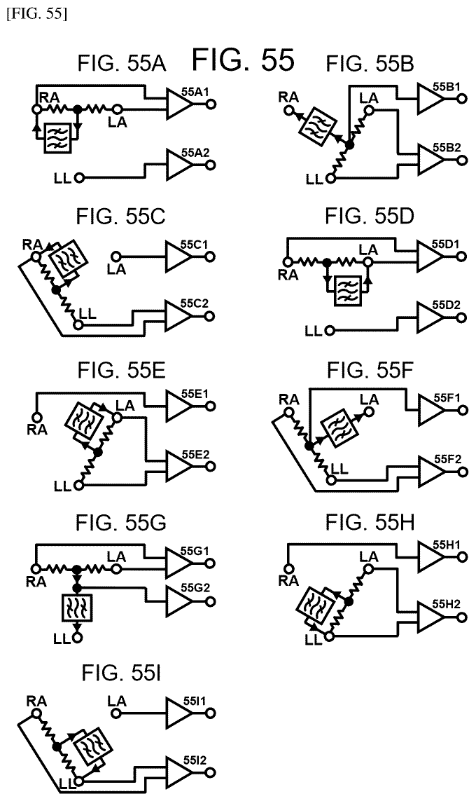

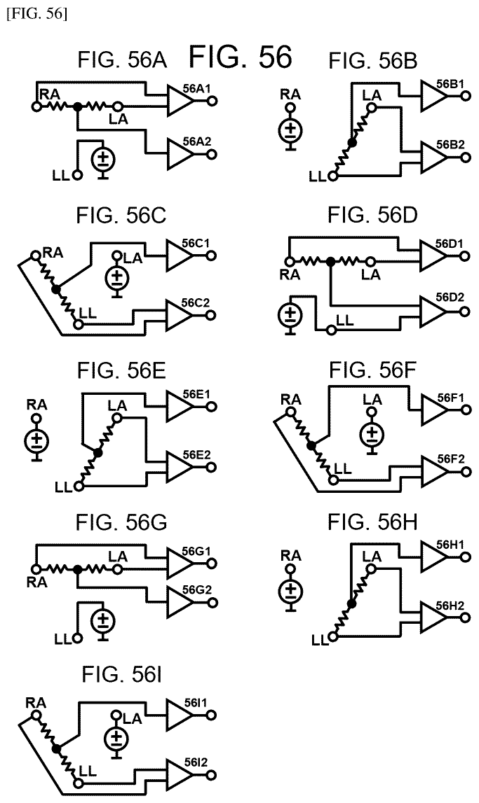

FIG. 56 illustrates embodiments of Group J, each including three electrodes; one electrode driver functioning as a constant voltage source (or constant voltage generator), the output of which is connected to one electrode; one voltage divider; and two amplifiers-one single-ended input amplifier connected to the center point of the voltage divider, and one differential amplifier connected across both ends of the voltage divider. The constant voltage source is connected to the electrode opposite the voltage divider. Embodiment 56F corresponds to the embodiment shown in FIG. 10.

Obtaining Lead II with Cancelled Power-Line Interference

A detailed analysis of power-line interference in Embodiment F1 shown in FIG. 41 will now be described. An important feature of Embodiment F1 in FIG. 41 is that the signal (−Lead I) obtained from amplifier 4110 contains power-line interference, whereas the Lead III signal obtained from amplifier 4120 contains almost no power-line interference—it is nearly eliminated. This was previously discussed with reference to Equation (27) and conclusion (ii) following that equation, using FIG. 42.

The voltage v₁ in Equation 26, which is measured with amplifier 4110, includes power-line interference. However, the voltage v₂ in Equation (27), which is measured with amplifier 4120, exhibits power-line interference that is almost completely attenuated. Accordingly, the power-line interference contained in the ECG lead signals calculated from these measured values can be expressed as follows. To highlight the concept, motion artifact is omitted from the following equations; only power-line interference is emphasized. Lead I is denoted simply as I. The ideal lead signal to be measured is vₐ, represented as I_{clean}=vₐ. I_{clean} denotes a Lead I signal free from power-line interference. The actually measured Lead I signal is represented as Iₘₑₐₛ. In the following expressions, (H(f)+1) is significantly large; thus, for convenience, the approximation iₙRₑₗ/(H(f)+1)=0 is used.

$${I}_{m e a s} = - {v}_{1} = {v}_{a} + {i}_{n} {R}_{e 1} = {I}_{clean} + {i}_{n} {R}_{e 1} \qquad ( Equation 47 )$$

$${III}_{meas} = {v}_{2} = {v}_{b} + {i}_{n} {R}_{e 1} / ( H ( f ) + 1 ) = {III}_{clean} + {i}_{n} {R}_{e 1} / ( H ( f ) + 1 ) = {III}_{clean} \qquad ( Equation 48 )$$

From the above, the following results are obtained. For calculated leads, the subscript “calc” is used. Using Equations (38) to (41) corresponding to the embodiment of FIG. 41, the following relationships hold:

$${II}_{c a l c} = {I}_{m e a s} + {III}_{m e a s} = {I}_{clean} + {i}_{n} {R}_{e 1} + {III}_{clean} \qquad ( Equation 49 )$$

$${aVR}_{c a l c} = - {I}_{m e a s} - {III}_{meas} / 2 = - {I}_{clean} - {i}_{n} {R}_{e 1} - {III}_{clean} / 2 = {aVR}_{clean} - {i}_{n} {R}_{e 1} \qquad ( Equation 50 )$$

$${aVL}_{c a l c} = ( {I}_{m e a s} - {III}_{m e a s} ) / 2 = ( {I}_{clean} + {i}_{n} {R}_{e 1} - {III}_{clean} ) / 2 = {aVL}_{clean} + {i}_{n} {R}_{e 1} / 2 \qquad ( Equation 51 )$$

$${aVF}_{c a l c} = {I}_{m e a s} / 2 + {III}_{m e a s} = ( {I}_{clean} + {i}_{n} {R}_{e 1} ) / 2 + {III}_{clean} = {aVF}_{clean} + {i}_{n} {R}_{e 1} / 2 \qquad ( Equation 52 )$$

The six limb-lead signals obtained above can thus be expressed as the sum of the desired ideal (“clean”) signals, which are free from power-line interference, and the interference components.

When the power-line interference components alone are extracted and summarized, the result is illustrated conceptually in FIG. 57. As shown in FIG. 57, the magnitude of power-line interference contained in each limb-lead ECG signal differs according to the lead.

This phenomenon depends on the configuration—that is, on the specific embodiment—of the electrocardiogram measurement apparatus. In the above example, the directly measured Lead III exhibits power-line interference that is almost completely eliminated, whereas Lead II still contains power-line interference.

Among the six limb-lead ECG signals, Lead II best represents the P-wave and QRS complex, and therefore has the greatest clinical importance. Accordingly, eliminating power-line interference from Lead II is of primary importance. The following describes embodiments for reducing power-line interference in Lead II.

To measure Lead II with minimal power-line interference using one of the embodiments of Group F, embodiments in which the output of the electrode driver is connected to the right-hand electrode or the left-leg electrode should not be used. To obtain a Lead II signal with reduced power-line interference, the electrode to which the output of the electrode driver is connected must contact the left-hand.

Embodiments for reducing the power-line interference of Lead II include Embodiment F3 shown in FIG. 43. The power-line interference in Embodiment F3 will now be analyzed. The Embodiment F1 analyzed above and Embodiment F3 analyzed here are symmetric. That is, the only difference between F1 and F3 is that the two electrodes connected to the input and output of the electrode driver (RA and LA) are interchanged. In Embodiment F3, Leads I and II are measured, and the following results are obtained. Again, the approximation iₙRₑₗ/(H(f)+1)=0 is applied for simplicity, and Equations (34) to (37) described previously are used.

$${I}_{m e a s} = {v}_{a} - {i}_{n} {R}_{e 1} = {I}_{clean} - {i}_{n} {R}_{e 1} \qquad ( Equation 53 )$$

$${II}_{meas} = {v}_{2} = {v}_{a} + {v}_{b} + {i}_{n} {R}_{e 1} / ( H ( f ) + 1 ) = {II}_{clean} + {i}_{n} {R}_{e 1} / ( H ( f ) + 1 ) = {II}_{clean} \qquad ( Equation 54 )$$

$${III}_{c a l c} = - {I}_{meas} + {II}_{m e a s} = - {I}_{clean} + {i}_{n} {R}_{e 1} + {II}_{clean} = {III}_{clean} + {i}_{n} {R}_{e 1} \qquad ( Equation 55 )$$

$${aVR}_{c a l c} = - ( {I}_{m e a s} + {II}_{meas} ) / 2 = - {I}_{clean} / 2 + {i}_{n} {R}_{e 1} / 2 - {II}_{clean} = {aVR}_{clean} + {i}_{n} {R}_{e 1} / 2 \qquad ( Equation 56 )$$

$${aVL}_{c a l c} = {I}_{m e a s} - {II}_{m e a s} / 2 = ( {I}_{clean} - {i}_{n} {R}_{e 1} - {II}_{clean} / 2 = {aVL}_{clean} - {i}_{n} {R}_{e 1} \qquad ( Equation 57 )$$

$${aVF}_{c a l c} = - {I}_{m e a s} / 2 + {II}_{m e a s} = - {I}_{clean} / 2 + {i}_{n} {R}_{e 1} / 2 + {II}_{clean} = {aVF}_{clean} + {i}_{n} {R}_{e 1} / 2 \qquad ( Equation 58 )$$

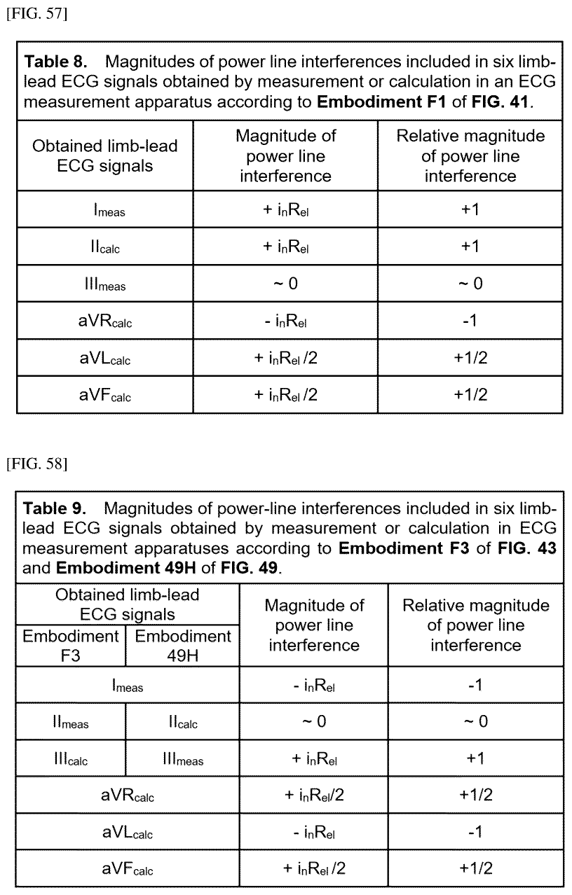

The results of power-line interference obtained from the above equations are summarized in FIG. 58. The key points to be noted from these equations are as follows: i) In the above example, the power-line interference in Lead II is almost completely eliminated. Lead II achieves a better signal-to-noise ratio than the other leads. ii) As described previously, depending on clothing or wearing conditions, the user may conveniently need to measure only Lead I. This Embodiment belongs to the group of embodiments suitable for measuring only Lead I. However, the Lead I signal measured in this embodiment includes power-line interference. iii) Lead signals other than Lead II also include power-line interference, but the level of interference is not significant due to the relatively low impedance Rₑₗ and the inherent characteristics of wearable devices. iv) By applying digital signal processing techniques for reducing power-line interference, all six lead signals can achieve a high signal-to-noise ratio.

The power-line interference in Embodiment 49H shown in FIG. 49 will now be analyzed. As described previously, the embodiments of FIG. 49 were referred to as Group H, and Embodiment 49H corresponds to Embodiment HH. The two single-ended input amplifiers included in Embodiment 49H respectively measure −Lead I and Lead III. Embodiment F1, analyzed above, also measures −Lead I and Lead III. However, as described below, the power-line interference analysis results for Embodiment HH differ from those for Embodiment F1.

$${I}_{m e a s} = {v}_{a} - {i}_{n} {R}_{e 1} = {I}_{clean} - {i}_{n} {R}_{e 1} \qquad ( Equation 59 )$$

$${III}_{meas} = {v}_{2} = {V}_{b} + {i}_{n} {R}_{e 1} = {III}_{clean} + {i}_{n} {R}_{e 1} \qquad ( Equation 60 )$$

Using these equations, the following results are obtained:

$${II}_{c a l c} = {I}_{m e a s} + {III}_{m e a s} = {I}_{clean} + {III}_{clean} = {II}_{clean} \qquad ( Equation 61 )$$

$${aVR}_{c a l c} = - {I}_{m e a s} - {III}_{meas} / 2 = - {I}_{clean} - {III}_{clean} / 2 + {i}_{n} {R}_{e 1} / 2 = {aVR}_{clean} + {i}_{n} {R}_{e 1} / 2 \qquad ( Equation 62 )$$

$${aVL}_{c a l c} = ( {I}_{m e a s} - {III}_{meas} ) / 2 = ( {I}_{clean} - {i}_{n} {R}_{e 1} - {III}_{clean} - {i}_{n} {R}_{e 1} ) / 2 = {aVL}_{clean} - {i}_{n} {R}_{e 1} \qquad ( Equation 63 )$$

$${aVF}_{c a l c} = {I}_{m e a s} / 2 + {III}_{meas} = ( {I}_{clean} - {i}_{n} {R}_{e 1} ) / 2 + {III}_{clean} + {i}_{n} {R}_{e 1} = {aVF}_{clean} + {i}_{n} {R}_{e 1} / 2 \qquad ( Equation 64 )$$

The results of power-line interference obtained from these equations are summarized in FIG. 58. These results are identical to those derived from Equations (49) through (54) and the analysis of Embodiment F3 shown in FIG. 58. Accordingly, although Embodiment 49H (i.e., Embodiment 51A) employs circuit components different from those of Embodiment F3, it is functionally equivalent to Embodiment F3. In Embodiment F3, Lead II is measured directly, whereas in Embodiment 49H, Lead II is obtained by calculation. Thus, these embodiments either measure or calculate Lead II with reduced power-line interference. Embodiment F3 and Embodiment 49H are therefore equivalent electrocardiographs in terms of their power-line interference reduction characteristics.

It should be noted from FIGS. 57 and 58 that each embodiment disclosed in the present invention may exhibit either identical or different power-line interference reduction characteristics among the six obtained ECG lead signals. Therefore, when selecting a specific embodiment, this point should not be overlooked.

As emphasized previously in connection with the embodiments including three electrodes (shown in FIGS. 4 through 10), when two or more differential amplifiers are used without connecting the output of one electrode driver to one electrode, power-line interference becomes so large that ECG signals cannot be properly measured. However, when the output of one electrode driver is connected to one electrode, two differential amplifiers may be used. This principle also applies to Group F. As described above, the use of differential amplifiers in such embodiments does not necessarily provide an advantage in reducing power-line interference. As shown above, the embodiments employing three electrodes exhibit equivalent power-line interference reduction effects.

The above principle for reducing the power-line interference of Lead II also applies to embodiments employing differential amplifiers. Therefore, in embodiments using three electrodes, the inclusion of differential amplifiers may be unnecessary.

On the other hand, examination of the above equations reveals that, to measure Lead I in which power-line interference is almost completely eliminated, the electrode connected to the output of the electrode driver must not contact the right hand or left hand. To obtain a Lead I signal with substantially eliminated power-line interference, the electrode connected to the output of the electrode driver must contact the left leg. As described earlier, in certain clothing or wearing conditions, it may be convenient to measure only Lead I. However, embodiments that measure Lead I with almost completely eliminated power-line interference cannot conveniently measure only Lead I.

As described above, embodiments of electrocardiogram measurement apparatuses using three or four electrodes to obtain six ECG limb-lead signals have been presented, with reference to figures and equations. The most important feature of the electrocardiogram measurement apparatus is the reduction of power-line interference, as explained in detail. In these electrocardiogram measurement apparatuses, the most essential condition for reducing power-line interference is that one electrode must be connected to the output terminal of one electrode driver. This has been emphasized and described in detail. Through detailed analysis, it has been shown that this is because the output terminal of the electrode driver supplies voltage and has a low output impedance.

The electrode driver may be, or may include, an amplifier, a band-pass filter, or a constant voltage source, as disclosed in the detailed embodiments. The input of the electrode driver may receive a signal from one electrode, or from a voltage divider composed of at least two resistors, as demonstrated in the embodiments. Embodiments employing one or more single-ended input amplifiers or one or more differential amplifiers have also been described. Further, embodiments for eliminating power-line interference from Lead II, which is medically the most important lead, have been disclosed. The equivalence and differences in the power-line interference characteristics among the embodiments for the six obtained ECG lead signals have been described in detail.

1. An electrocardiogram measurement apparatus comprising:
a first electrode and a second electrode configured to respectively receive a first electrocardiogram voltage and a second electrocardiogram voltage of a first human body part and a second human body part in contact therewith;
two amplifiers configured to respectively receive the first electrocardiogram voltage and the second electrocardiogram voltage from the first electrode and the second electrode;
an electrode driver configured to output a driving voltage;
a third electrode configured to contact a third human body part and transmit a third electrocardiogram voltage to an input of the electrode driver;
an AD converter connected to output terminals of the two amplifiers and configured to convert output signals of the two amplifiers into two digital signals;
a microcontroller configured to receive the two digital signals from the AD converter; and
a communication element configured to transmit the two digital signals;
wherein:
the microcontroller is supplied with power from a battery;
controls the AD converter and the communication element; and
each of the two amplifiers, respectively and simultaneously, receives and amplifies one electrocardiogram voltage.

2. The electrocardiogram measurement apparatus according to claim 1, wherein an output of the electrode driver is connected to the second electrode.

3. The electrocardiogram measurement apparatus according to claim 1, further comprising a fourth electrode connected to an output of the electrode driver, wherein the fourth electrode is installed adjacent to one electrode among the three electrodes, so that the fourth electrode and the adjacent electrode among the three electrodes contact the same human body part.

4. The electrocardiogram measurement apparatus according to claim 1, wherein the electrode connected to the input of the electrode driver and an electrode connected to an output of the electrode driver contact a human body part.

5. The electrocardiogram measurement apparatus according to claim 1, wherein the electrode connected to the input of the electrode driver and an electrode connected to an output of the electrode driver contact different human body parts.

6. The electrocardiogram measurement apparatus according to claim 1, wherein the input of the electrode driver is connected to a central point of one voltage divider and receives signals from two electrodes among the first, second, and third electrodes.

7. The electrocardiogram measurement apparatus according to claim 1, wherein the input of the electrode driver is connected to a central point of three resistors and receives signals from the first, second, and third electrodes.

8. The electrocardiogram measurement apparatus according to claim 1, wherein at least one of the two amplifiers of the electrocardiogram measurement apparatus is a single-ended input amplifier.

9. The electrocardiogram measurement apparatus according to claim 1, wherein at least one of the two amplifiers of the electrocardiogram measurement apparatus is a differential amplifier.

10. The electrocardiogram measurement apparatus according to claim 1, wherein the electrode driver is an amplifier.

11. The electrocardiogram measurement apparatus according to claim 1, wherein the electrode driver is a band-pass filter.

12. The electrocardiogram measurement apparatus according to claim 1, wherein the electrode driver is a constant voltage source, and the third electrode is connected to an output of the constant voltage source instead of the input of the electrode driver.

13. The electrocardiogram measurement apparatus according to claim 1, wherein the electrocardiogram measurement apparatus obtains six electrocardiogram lead signals.

14. The electrocardiogram measurement apparatus according to claim 13, wherein the electrocardiogram measurement apparatus displays the six electrocardiogram lead signals on a screen of a smartphone.

15. The electrocardiogram measurement apparatus according to claim 1, wherein one electrode of the electrocardiogram measurement apparatus contacts a left leg.

16. The electrocardiogram measurement apparatus according to claim 1, further comprising a current detector, wherein the electrocardiogram measurement apparatus starts an electrocardiogram measurement when the current detector detects contact of both hands.

17. The electrocardiogram measurement apparatus according to claim 1, further comprising a photoplethysmograph, wherein the electrocardiogram measurement apparatus starts an electrocardiogram measurement when the photoplethysmograph detects an occurrence of an arrhythmia and generates an alarm.

18. The electrocardiogram measurement apparatus according to claim 1, wherein the electrocardiogram measurement apparatus stores, in a memory of the electrocardiogram measurement apparatus, a parameter indicating a hand on which the electrocardiogram measurement apparatus is worn.

19. The electrocardiogram measurement apparatus according to claim 18, wherein the electrocardiogram measurement apparatus selects calculation formulas according to the parameter indicating the wearing hand stored in the memory and calculates and obtains additional electrocardiogram lead signals.

20. The electrocardiogram measurement apparatus according to claim 14, wherein among the six electrocardiogram lead signals, Lead II has the least power line interference.
